SEQUENCE LISTING (174 sequences)

SEQ ID NO: 1 - protein, 186 aa, Homo sapiens [sequence omitted]

SEQ ID NO: 2 - protein, 116 aa, Artificial Sequence
  Mouse 7H11 VH
QVQLQQSGPELVKPGASVKMSCKASGYTFTRYYIHWMNQRPGQGLEWIGWIYPGDGSTKYNEKFKGKTTLTADTSSSTAYMLLSSLTSEDSAIYFCAREYGNYDYWGQGTTVTVSS

SEQ ID NO: 3 - protein, 113 aa, Artificial Sequence
  Mouse 7H11 VL
DIVMTQSPSSLSVSAGEKVTMSCKSSQSLLNSGNQKNYLAWYQQKPGQPPKLLIYGASTRESGVPDRFTGSGSGTDFTLTISSVQAEDLAVYYCQNDHSYPYTFGGGTKLEIK

SEQ ID NO: 4 - protein, 98 aa, Artificial Sequence
  IGHV1-3*01
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 5 - protein, 98 aa, Artificial Sequence
  IGHV1-2*02
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAR

SEQ ID NO: 6 - protein, 98 aa, Artificial Sequence
  IGHV1-46*01
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 7 - protein, 98 aa, Artificial Sequence
  IGHV1-8*01
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 8 - protein, 101 aa, Artificial Sequence
  IGKV4-1*01
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 9 - protein, 96 aa, Artificial Sequence
  IGKV3D-7*01
EIVMTQSPATLSLSPGERATLSCRASQSVSSSYLSWYQQKPGQAPRLLIYGASTRATGIPARFSGSGSGTDFTLTISSLQPEDFAVYYCQQDYNLP

SEQ ID NO: 10 - protein, 95 aa, Artificial Sequence
  IGKV3D-15*01
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 11 - protein, 96 aa, Artificial Sequence
  IGKV3-20*01
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 12 - protein, 446 aa, Artificial Sequence
  Humanized 7H11-VH1
QVQLVQSGAEVKKPGASVKVSCKASGYTFTRYYMHWVRQAPGQGLEWMGWIYPGDGSTKYSQKFQGRVTITRDTSASTAYMELSSLRSEDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 446 aa, Artificial Sequence
  Humanized 7H11-VH2
QVQLVQSGAEVKKPGASVKVSCKASGYTFTRYYMHWVRQAPGQGLEWMGWIYPGDGSTNYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 446 aa, Artificial Sequence
  Humanized 7H11-VH3
QVQLVQSGAEVKKPGASVKVSCKASGYTFTRYYMHWVRQAPGQGLEWMGIIYPGDGSTSYAQKFQGRVTMTRDTSTSTVYMELSSLRSEDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 446 aa, Artificial Sequence
  Humanized 7H11-VH4
QVQLVQSGAEVKKPGASVKVSCKASGYTFTRYYINWVRQATGQGLEWMGWIYPGDGSTGYAQKFQGRVTMTRNTSISTAYMELSSLRSEDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 220 aa, Artificial Sequence
  Humanized 7H11-VL1
DIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 220 aa, Artificial Sequence
  Humanized 7H11-VL2
EIVMTQSPPTLSLSPGERVTLSCRASQSLLNSGNQKNYLSWYQQKPGQAPRLLIYGASTRATGIPARFSGSGSGTDFTLTISSLQPEDFAVYYCQNDHSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 220 aa, Artificial Sequence
  Humanized 7H11-VL3
EIVMTQSPATLSVSPGERATLSCRASQSLLNSGNQKNYLAWYQQKPGQAPRLLIYGASTRATGIPARFSGSGSGTEFTLTISSLQSEDFAVYYCQNDHSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 220 aa, Artificial Sequence
  Humanized 7H11-VL4
EIVLTQSPGTLSLSPGERATLSCRASQSLLNSGNQKNYLAWYQQKPGQAPRLLIYGASSRATGIPDRFSGSGSGTDFTLTISRLEPEDFAVYYCQNDHSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 446 aa, Artificial Sequence
  Humanized 7H11-VH2 V37M
QVQLVQSGAEVKKPGASVKVSCKASGYTFTRYYMHWMRQAPGQGLEWMGWIYPGDGSTNYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 446 aa, Artificial Sequence
  Humanized 7H11-VH2 N58K
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 446 aa, Artificial Sequence
  Humanized 7H11-VH2 A60N
QVQLVQSGAEVKKPGASVKVSCKASGYTFTRYYMHWVRQAPGQGLEWMGWIYPGDGSTNYNQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 446 aa, Artificial Sequence
  Humanized 7H11-VH2 Q61E
QVQLVQSGAEVKKPGASVKVSCKASGYTFTRYYMHWVRQAPGQGLEWMGWIYPGDGSTNYAEKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 446 aa, Artificial Sequence
  Humanized 7H11-VH2 D85E
QVQLVQSGAEVKKPGASVKVSCKASGYTFTRYYMHWVRQAPGQGLEWMGWIYPGDGSTNYAQKFQGRVTMTRDTSISTAYMELSRLRSEDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 446 aa, Artificial Sequence
  Humanized 7H11-VH2 V89I
QVQLVQSGAEVKKPGASVKVSCKASGYTFTRYYMHWVRQAPGQGLEWMGWIYPGDGSTNYAQKFQGRVTMTRDTSISTAYMELSRLRSDDTAIYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 446 aa, Artificial Sequence
  Humanized 7H11-VH2 Y91F
QVQLVQSGAEVKKPGASVKVSCKASGYTFTRYYMHWVRQAPGQGLEWMGWIYPGDGSTNYAQKFQGRVTMTRDTSISTAYMELSRLRSDDTAVYFCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 446 aa, Artificial Sequence
  Humanized 7H11-VH2 N58K-D54E
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 446 aa, Artificial Sequence
  Humanized 7H11-VH2 N58K-D54S
QVQLVQSGAEVKKPGASVKVSCKASGYTFTRYYMHWVRQAPGQGLEWMGWIYPGSGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 446 aa, Artificial Sequence
  Humanized 7H11-VH2 N58K-D54T
QVQLVQSGAEVKKPGASVKVSCKASGYTFTRYYMHWVRQAPGQGLEWMGWIYPGTGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 446 aa, Artificial Sequence
  Humanized 7H11-VH2 N58K-G55A
QVQLVQSGAEVKKPGASVKVSCKASGYTFTRYYMHWVRQAPGQGLEWMGWIYPGDASTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 117 aa, Artificial Sequence
  Mouse 2H6 VH
DVQLQESGPGLVKPSQSLSLTCTVTGYSITSDYAWNWIRQFPGNKLEWMGYISYSGSTAYNPSLKSRISITRDTSKNQFFLQLNSVTTEDTATYYCARGPYNYLAYWGQGTLVTVSS

SEQ ID NO: 32 - protein, 111 aa, Artificial Sequence
  Mouse 2H6 VL
DIVLTQSPASLAVSLGQRATISCRASESVDNYGISFMNWFQQKPGQPPKLLIYAASNQKSGVPARFSGSGSGTDFSLNIHPMEEDDTAMYFCQQSKEVPLTFGAGTKLEIK

SEQ ID NO: 33 - protein, 98 aa, Artificial Sequence
  IGHV3-23*01
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 34 - protein, 95 aa, Artificial Sequence
  IGKV1-16*01
DIQMTQSPSSLSASVGDRVTITCRASQGISNYLAWFQQKPGKAPKLLIYAASSLQSGVPSRFSGSGSGTDFTLTISSLQPEDFATYYCQQYNSYP

SEQ ID NO: 35 - protein, 475 aa, Artificial Sequence
  Mouse 2H6 scFv-Fc
DVQLQESGPGLVKPSQSLSLTCTVTGYSITSDYAWNWIRQFPGNKLEWMGYISYSGSTAYNPSLKSRISITRDTSKNQFFLQLNSVTTEDTATYYCARGPYNYLAYWGQGTLVTVSSGGGGSGGGGSGGGGSDIVLTQSPASLAVSLGQRATISCRASESVDNYGISFMNWFQQKPGQPPKLLIYAASNQKSGVPARFSGSGSGTDFSLNIHPMEEDDTAMYFCQQSKEVPLTFGAGTKLEIKGGGGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 475 aa, Artificial Sequence
  2H6 scFv1-Fc
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIKGGGGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 475 aa, Artificial Sequence
  2H6 scFv2-Fc
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIKGGGGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 693 aa, Artificial Sequence
  Tetra-1 HC
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIK

SEQ ID NO: 39 - protein, 693 aa, Artificial Sequence
  Tetra-6 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIL

SEQ ID NO: 40 - protein, 693 aa, Artificial Sequence
  Tetra-2 HC
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIK

SEQ ID NO: 41 - protein, 695 aa, Artificial Sequence
  Tetra-3 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSGGGGSGGGGSGGGGSDIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGQGTKLEIK

SEQ ID NO: 42 - protein, 694 aa, Artificial Sequence
  Tetra-4 HC
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSGGGGSGGGGSGGGGSDIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGQGTKLEIK

SEQ ID NO: 43 - protein, 694 aa, Artificial Sequence
  Tetra-5 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIK

SEQ ID NO: 44 - protein, 693 aa, Artificial Sequence
  Tetra-7 HC
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIK

SEQ ID NO: 45 - protein, 693 aa, Artificial Sequence
  Tetra-8 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 46 - protein, 693 aa, Artificial Sequence
  Tetra-9 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWVRQAPGKGLEWVSYISYSGSTAYNPSLKSRFTISRDTSKNTLYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIL

SEQ ID NO: 47 - protein, 693 aa, Artificial Sequence
  Tetra-10 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWVRQAPGKCLEWVSYISYSGSTAYNPSLKSRFTISRDTSKNTLYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 48 - protein, 693 aa, Artificial Sequence
  Tetra-11 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWVRQAPGKGLEWVGYISYSGSTAYNPSLKSRFTISRDTSKNTLYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIL

SEQ ID NO: 49 - protein, 693 aa, Artificial Sequence
  Tetra-12 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWVRQAPGKCLEWVGYISYSGSTAYNPSLKSRFTISRDTSKNTLYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 50 - DNA, 41 bp, Artificial Sequence
  Primer Mix VH - BACK_1
gtgatcgccatggcgtcgaccgakgtrmagcttcaggagtc

SEQ ID NO: 51 - DNA, 41 bp, Artificial Sequence
  Primer Mix VH - BACK_2
gtgatcgccatggcgtcgaccgaggtbcagctbcagcagtc

SEQ ID NO: 52 - DNA, 41 bp, Artificial Sequence
  Primer Mix VH - BACK_3
gtgatcgccatggcgtcgacccaggtgcagctgaagsartc

SEQ ID NO: 53 - DNA, 41 bp, Artificial Sequence
  Primer Mix VH - BACK_4
gtgatcgccatggcgtcgaccgaggtccarctgcaacartc

SEQ ID NO: 54 - DNA, 41 bp, Artificial Sequence
  Primer Mix VH - BACK_5
gtgatcgccatggcgtcgacccaggtycagctbcagcartc

SEQ ID NO: 55 - DNA, 41 bp, Artificial Sequence
  Primer Mix VH - BACK_6
gtgatcgccatggcgtcgacccaggtycarctgcagcartc

SEQ ID NO: 56 - DNA, 41 bp, Artificial Sequence
  Primer Mix VH - BACK_7
gtgatcgccatggcgtcgacccaggtccacgtgaagcartc

SEQ ID NO: 57 - DNA, 41 bp, Artificial Sequence
  Primer Mix VH - BACK_8
gtgatcgccatggcgtcgaccgaggtgaasstggtggartc

SEQ ID NO: 58 - DNA, 41 bp, Artificial Sequence
  Primer Mix VH - BACK_9
gtgatcgccatggcgtcgaccgavgtgawgstggtggagtc

SEQ ID NO: 59 - DNA, 41 bp, Artificial Sequence
  Primer Mix VH - BACK_10
gtgatcgccatggcgtcgaccgaggtgcagstggtggartc

SEQ ID NO: 60 - DNA, 41 bp, Artificial Sequence
  Primer Mix VH - BACK_11
gtgatcgccatggcgtcgaccgakgtgcamctggtggartc

SEQ ID NO: 61 - DNA, 41 bp, Artificial Sequence
  Primer Mix VH - BACK_12
gtgatcgccatggcgtcgaccgaggtgaagctgatggartc

SEQ ID NO: 62 - DNA, 41 bp, Artificial Sequence
  Primer Mix VH - BACK_13
gtgatcgccatggcgtcgaccgaggtgcarcttgttgartc

SEQ ID NO: 63 - DNA, 41 bp, Artificial Sequence
  Primer Mix VH - BACK_14
gtgatcgccatggcgtcgaccgargtraagcttctcgartc

SEQ ID NO: 64 - DNA, 41 bp, Artificial Sequence
  Primer Mix VH - BACK_15
gtgatcgccatggcgtcgaccgaagtgaarsttgaggartc

SEQ ID NO: 65 - DNA, 41 bp, Artificial Sequence
  Primer Mix VH - BACK_16
gtgatcgccatggcgtcgacccaggttactctraaasartc

SEQ ID NO: 66 - DNA, 41 bp, Artificial Sequence
  Primer Mix VH - BACK_17
gtgatcgccatggcgtcgacccaggtccaactvcagcarcc

SEQ ID NO: 67 - DNA, 41 bp, Artificial Sequence
  Primer Mix VH - BACK_18
gtgatcgccatggcgtcgaccgatgtgaacttggaasartc

SEQ ID NO: 68 - DNA, 41 bp, Artificial Sequence
  Primer Mix VH - BACK_19
gtgatcgccatggcgtcgaccgaggtgaaggtcatcgartc

SEQ ID NO: 69 - DNA, 38 bp, Artificial Sequence
  Primer Mix VH - FOR_1
cctccaccactcgagcccgaggaaacggtgaccgtggt

SEQ ID NO: 70 - DNA, 38 bp, Artificial Sequence
  Primer Mix VH - FOR_2
cctccaccactcgagcccgaggagactgtgagagtggt

SEQ ID NO: 71 - DNA, 38 bp, Artificial Sequence
  Primer Mix VH - FOR_3
cctccaccactcgagcccgcagagacagtgaccagagt

SEQ ID NO: 72 - DNA, 38 bp, Artificial Sequence
  Primer Mix VH - FOR_4
cctccaccactcgagcccgaggagacggtgactgaggt

SEQ ID NO: 73 - DNA, 35 bp, Artificial Sequence
  Primer Mix VL - BACK_1
ggcggtggcgctagcgayatccagctgactcagcc

SEQ ID NO: 74 - DNA, 35 bp, Artificial Sequence
  Primer Mix VL - BACK_2
ggcggtggcgctagccaaattgttctcacccagtc

SEQ ID NO: 75 - DNA, 35 bp, Artificial Sequence
  Primer Mix VL - BACK_3
ggcggtggcgctagcgayattgtgmtmactcagtc

SEQ ID NO: 76 - DNA, 35 bp, Artificial Sequence
  Primer Mix VL - BACK_4
ggcggtggcgctagcgayattgtgytracacagtc

SEQ ID NO: 77 - DNA, 35 bp, Artificial Sequence
  Primer Mix VL - BACK_5
ggcggtggcgctagcgayattgtratgacmcagtc

SEQ ID NO: 78 - DNA, 35 bp, Artificial Sequence
  Primer Mix VL - BACK_6
ggcggtggcgctagcgayattmagatramccagtc

SEQ ID NO: 79 - DNA, 35 bp, Artificial Sequence
  Primer Mix VL - BACK_7
ggcggtggcgctagcgayattcagatgaydcagtc

SEQ ID NO: 80 - DNA, 35 bp, Artificial Sequence
  Primer Mix VL - BACK_8
ggcggtggcgctagcgayatycagatgacacagac

SEQ ID NO: 81 - DNA, 35 bp, Artificial Sequence
  Primer Mix VL - BACK_9
ggcggtggcgctagcgayattgttctcawccagtc

SEQ ID NO: 82 - DNA, 35 bp, Artificial Sequence
  Primer Mix VL - BACK_10
ggcggtggcgctagcgayattgwgctsacccaatc

SEQ ID NO: 83 - DNA, 35 bp, Artificial Sequence
  Primer Mix VL - BACK_11
ggcggtggcgctagcgayattstratgacccartc

SEQ ID NO: 84 - DNA, 35 bp, Artificial Sequence
  Primer Mix VL - BACK_12
ggcggtggcgctagcgayrttktgatgacccarac

SEQ ID NO: 85 - DNA, 35 bp, Artificial Sequence
  Primer Mix VL - BACK_13
ggcggtggcgctagcgayattgtgatgacbcagkc

SEQ ID NO: 86 - DNA, 35 bp, Artificial Sequence
  Primer Mix VL - BACK_14
ggcggtggcgctagcgayattgtgataacycagga

SEQ ID NO: 87 - DNA, 35 bp, Artificial Sequence
  Primer Mix VL - BACK_15
ggcggtggcgctagcgayattgtgatgacccagwt

SEQ ID NO: 88 - DNA, 35 bp, Artificial Sequence
  Primer Mix VL - BACK_16
ggcggtggcgctagcgayattgtgatgacacaacc

SEQ ID NO: 89 - DNA, 35 bp, Artificial Sequence
  Primer Mix VL - BACK_17
ggcggtggcgctagcgayattttgctgactcagtc

SEQ ID NO: 90 - DNA, 35 bp, Artificial Sequence
  Primer Mix VL - BACK_18
ggcggtggcgctagcgaaacaactgtgacccagtc

SEQ ID NO: 91 - DNA, 35 bp, Artificial Sequence
  Primer Mix VL - BACK_19
ggcggtggcgctagcgaaaatgtkctsacccagtc

SEQ ID NO: 92 - DNA, 38 bp, Artificial Sequence
  Primer Mix VL - BACK_20
ggcggtggcgctagccaggctgttgtgactcaggaatc

SEQ ID NO: 93 - DNA, 36 bp, Artificial Sequence
  Primer Mix VL - FOR_1
atgctgacgcggccgcacgtttkatttccagcttgg

SEQ ID NO: 94 - DNA, 36 bp, Artificial Sequence
  Primer Mix VL - FOR_2
atgctgacgcggccgcacgttttatttccaactttg

SEQ ID NO: 95 - DNA, 36 bp, Artificial Sequence
  Primer Mix VL - FOR_3
atgctgacgcggccgcacgtttcagctccagcttgg

SEQ ID NO: 96 - DNA, 36 bp, Artificial Sequence
  Primer Mix VL - FOR_4
atgctgacgcggccgcacctaggacagtcagtttgg

SEQ ID NO: 97 - DNA, 17 bp, Artificial Sequence
  M13-Fwd
gtaaaacgacggccagt

SEQ ID NO: 98 - DNA, 16 bp, Artificial Sequence
  M13-Rev
aacagctatgaccatg

SEQ ID NO: 99 - DNA, 19 bp, Artificial Sequence
  T7
taatacgactcactatagg

SEQ ID NO: 100 - DNA, 19 bp, Artificial Sequence
  SP6
gatttaggtgacactatag

SEQ ID NO: 101 - protein, 444 aa, Artificial Sequence
  11D4 IgG2 HC
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 102 - protein, 214 aa, Artificial Sequence
  11D4 LC
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 451 aa, Artificial Sequence
  9B12 IgG1 HC
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, Artificial Sequence
  9B12 LC
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 452 aa, Artificial Sequence
  106-222 IgG1 HC
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 214 aa, Artificial Sequence
  106-222 LC
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 447 aa, Artificial Sequence
  1A7 IgG1 HC
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 214 aa, Artificial Sequence
  1A7 LC
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 451 aa, Artificial Sequence
  pab1949 IgG1 HC
EVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 219 aa, Artificial Sequence
  pab1949 LC
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 468 aa, Artificial Sequence
  Tetra-hz1D10v1 HC
EVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKPGGSGGSEVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKPGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 470 aa, Artificial Sequence
  Tetra-hzG3V9 HC
EVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKPGGSGGSEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKPGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 594 aa, Artificial Sequence
  Hexa-hz1D10v1 HC
EVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKPGGSGGSEVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKPGGSGGSEVQLLESGGGEVQPGGSLRLSCAASGFTFSDAFMYWVRQAPGKGLEWVSSISNRGLKTAYAESVKGRFTISRDNAKNTLYLQMSSLRAEDTAVYYCSRDVDGDFRGQGTLVTVKPGGGGTGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 597 aa, Artificial Sequence
  Hexa-hzG3V9 HC
EVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKPGGSGGSEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKPGGSGGSEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKPGGGGTGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 447 aa, Artificial Sequence
  2H6 IgG1 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 218 aa, Artificial Sequence
  2H6 LC
DIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 447 aa, Artificial Sequence
  2H6 IgG1 LALA HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 475 aa, Artificial Sequence
  2H6 scFv-Fc LALA HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTMVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGQGTKVEIKGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 446 aa, Artificial Sequence; identical to SEQ ID NO: 27
  7H11_v8 IgG1 HC

SEQ ID NO: 120 - protein, 446 aa, Artificial Sequence
  7H11 IgG1 LALA HC
QVQLVQSGAEVKKPGASVKVSCKASGYTFTRYYMHWVRQAPGQGLEWMGWIYPGDGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 187 aa, Rattus norvegicus
LNCVKDTYPSGHKCCRECQPGHGMVSRCDHTRDTVCHPCEPGFYNEAVNYDTCKQCTQCNHRSGSELKQNCTPTEDTVCQCRPGTQPRQDSSHKLGVDCVPCPPGHFSPGSNQACKPWTNCTLSGKQIRHPASNSLDTVCEDRSLLATLLWETQRTTFRPTTVPSTTVWPRTSQLPSTPTLVAPEGP

SEQ ID NO: 122 - protein, 186 aa, Macaca fascicularis
LHCVGDTYPSNDRCCQECRPGNGMVSRCNRSQNTVCRPCGPGFYNDVVSAKPCKACTWCNLRSGSERKQPCTATQDTVCRCRAGTQPLDSYKPGVDCAPCPPGHFSPGDNQACKPWTNCTLAGKHTLQPASNSSDAICEDRDPPPTQPQETQGPPARPTTVQPTEAWPRTSQRPSTRPVEVPRGPA

SEQ ID NO: 123 - protein, 424 aa, Artificial Sequence
  hsOX40(ECD)-Fc-His
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAGSGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSAHHHHHH

SEQ ID NO: 124 - protein, 416 aa, Artificial Sequence
  cynoOX40R(ECD)-Fc
LHCVGDTYPSNDRCCQECRPGNGMVSRCNRSQNTVCRPCGPGFYNDVVSAKPCKACTWCNLRSGSERKQPCTATQDTVCRCRAGTQPLDSYKPGVDCAPCPPGHFSPGDNQACKPWTNCTLAGKHTLQPASNSSDAICEDRDPPPTQPQETQGPPARPTTVQPTEAWPRTSQRPSTRPVEVPRGPAQASGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 421 aa, Artificial Sequence
  rnOX40(ECD)-Fc
VTVKLNCVKDTYPSGHKCCRECQPGHGMVSRCDHTRDTVCHPCEPGFYNEAVNYDTCKQCTQCNHRSGSELKQNCTPTEDTVCQCRPGTQPRQDSSHKLGVDCVPCPPGHFSPGSNQACKPWTNCTLSGKQIRHPASNSLDTVCEDRSLLATLLWETQRTTFRPTTVPSTTVWPRTSQLPSTPTLVAPEGPQASGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 415 aa, Artificial Sequence
  chiOX40R(ECD)-HHHR-Fc
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGSNQACKPWTNCTLSGKQIRHPASNSLDTVCEDRSLLATLLWETQRTTFRPTTVPSTTVWPRTSQLPSTPTLVAPEGPGGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 418 aa, Artificial Sequence
  chiOX40R(ECD)-HHRH-Fc
LHCVGDTYPSNDRCCHECRPGNGMVSRCSRSQNTVCRPCGPGFYNDVVSSKPCKPCTWCNLRSGSERKQLCTATQDTVCRCRPGTQPRQDSSHKLGVDCVPCPPGHFSPGDNQACKPWTNCTLAGKHTLQPASNSSDAICEDRDPPATQPQETQGPPARPITVQPTEAWPRTSQGPSTRPVEVPGGRAGGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 428 aa, Artificial Sequence
  chiOX40R(ECD)-HHRR-Fc
LHCVGDTYPSNDRCCHECRPGNGMVSRCSRSQNTVCRPCGPGFYNDVVSSKPCKPCTWCNLRSGSERKQLCTATQDTVCQCRPGTQPRQDSSHKLGVDCVPCPPGHFSPGSNQACKPWTNCTLSGKQIRHPASNSLDTVCEDRSLLATLLWETQRTTFRPTTVPSTTVWPRTSQLPSTPTLVAPEGPAAALEVLFQGPLGSGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 428 aa, Artificial Sequence
  chiOX40R(ECD)-HRRR-Fc
LHCVGDTYPSNDRCCHECRPGNGMVSRCSRSQNTVCHPCEPGFYNEAVNYDTCKQCTQCNHRSGSELKQNCTPTEDTVCQCRPGTQPRQDSSHKLGVDCVPCPPGHFSPGSNQACKPWTNCTLSGKQIRHPASNSLDTVCEDRSLLATLLWETQRTTFRPTTVPSTTVWPRTSQLPSTPTLVAPEGPAAALEVLFQGPLGSGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 430 aa, Artificial Sequence
  chiOX40R(ECD)-RRHH-Fc
VTVKLNCVKDTYPSGHKCCRECQPGHGMVSRCDHTRDTVCHPCEPGFYNEAVNYDTCKQCTQCNHRSGSELKQNCTPTEDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAAALEVLFQGPLGSGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 432 aa, Artificial Sequence
  chiOX40R(ECD)-RHRR-Fc
VTVKLNCVKDTYPSGHKCCRECQPGHGMVSRCDHTRDTVCRPCGPGFYNDVVSSKPCKPCTWCNLRSGSERKQLCTATQDTVCQCRPGTQPRQDSSHKLGVDCVPCPPGHFSPGSNQACKPWTNCTLSGKQIRHPASNSLDTVCEDRSLLATLLWETQRTTFRPTTVPSTTVWPRTSQLPSTPTLVAPEGPAAALEVLFQGPLGSGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 693 aa, Artificial Sequence
  Tetra-13 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 133 - protein, 695 aa, Artificial Sequence
  Tetra-14 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLVQSGAEVKKPGASVKVSCKASGYTFTRYYMHWVRQAPGQCLEWMGWIYPGEGSTKYAQKFQGRVTMTRDTSISTAYMELSRLRSDDTAVYYCAREYGNYDYWGQGTLVTVSSGGGGSGGGGSGGGGSDIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGCGTKLEIL

SEQ ID NO: 134 - protein, 694 aa, Artificial Sequence
  Tetra-15 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 135 - protein, 694 aa, Artificial Sequence
  Tetra-16 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLVQSGAEVKKPGASVKVSCKASGYTFTRYYMHWVRQAPGQCLEWMGWIYPGEGSTKYAQKFQGRVTMTRDTSISTAYMELSRLRSDDTAVYYCAREYGNYDYWGQGTLVTVSSGGGGSGGGGSGGGGSDIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGCGTKLEIL

SEQ ID NO: 136 - protein, 669 aa, Artificial Sequence
  Tetra-17 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 137 - protein, 671 aa, Artificial Sequence
  Tetra-18 HC
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 138 - protein, 693 aa, Artificial Sequence
  Tetra-19 HC1
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMSSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 139 - protein, 669 aa, Artificial Sequence
  Tetra-19 HC2
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGGGGGTQVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 140 - protein, 695 aa, Artificial Sequence
  Tetra-20 HC1
EVQLVESGGGLVQPGGSLRLSCAVSGYSITSDYAWNWIRQAPGKGLEWMGYISYSGSTAYNPSLKSRITISRDTSKNTFYLQMSSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPAVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGGGGGTQVQLVQSGAEVKKPGASVKVSCKASGYTFTRYYMHWVRQAPGQCLEWMGWIYPGEGSTKYAQKFQGRVTMTRDTSISTAYMELSRLRSDDTAVYYCAREYGNYDYWGQGTLVTVSSGGGGSGGGGSGGGGSDIVMTQSPDSLAVSLGERATINCKSSQSLLNSGNQKNYLAWYQQKPGQPPKLLIYGASTRESGVPDRFSGSGSGTDFTLTISSLQAEDVAVYYCQNDHSYPYTFGCGTKLEIL

SEQ ID NO: 141 - protein, 671 aa, Artificial Sequence
  Tetra-20 HC2
EVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPGGGGGTEVQLVESGGGLVQPGGSLRLSCAVSGYSITSDYAWNWIRQAPGKGLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 142 - protein, 700 aa, Artificial Sequence
  Tetra-21 HC2
EVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIKRTGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGGGGGTQVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 143 - protein, 684 aa, Artificial Sequence
  Tetra-22 HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 144 - protein, 214 aa, Artificial Sequence
  Tetra-22 LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 446 aa, Artificial Sequence
  Tri-8 HC2
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESQGSFALSSRLRVDKSRWQQGNVFSCSVMHEALHNHYTQKSLSLSPGK

SEQ ID NO: 146 - protein, 694 aa, Artificial Sequence
  1A7_2H6_8 HC
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 147 - protein, 699 aa, Artificial Sequence
  106-222_2H6_8 HC
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSGGGGSGGGGSGGGGSDIQLTQSPSSLSASVGDRVTITCRASESVDNYGISFMNWFQQKPGKAPKLLIYAASNQKSGVPSRFSGSGSGTDFTLTISSLQPEDFATYFCQQSKEVPLTFGCGTKVEIL

SEQ ID NO: 148 - protein, 699 aa, Artificial Sequence
  7H11_1949_8 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSGGGGSGGGGSGGGGSTDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIK

SEQ ID NO: 149 - protein, 701 aa, Artificial Sequence
  11D4_1949 HC
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSGGGGSGGGGSGGGGSTDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIK

SEQ ID NO: 150 - protein, 720 aa, Artificial Sequence
  1A7_1949 HC
METDTLLLWVLLLWVPGSTGEVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSGGGGSGGGGSGGGGSTDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIK

SEQ ID NO: 151 - protein, 704 aa, Artificial Sequence
  9B12_1949 HC
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSGGGGSGGGGSGGGGSTDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIK

SEQ ID NO: 152 - protein, 705 aa, Artificial Sequence
  106-222_1949 HC
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLVESGGGLVQPGGSLKLSCAASGFTFSGSAMHWVRQASGKCLEWVGRIRSKANSYATAYAASVKGRFTISRDDSKNTAYLQMNSLKTEDTAVYYCTSGIYDSSGYDYWGQGTLVTVSSGGGGSGGGGSGGGGSTDIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPLTFGCGTKVEIK

SEQ ID NO: 153 - protein, 693 aa, Artificial Sequence
  7H11_9B12_8 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTQVQLQESGPGLVKPSQTLSLTCAVYGGSFSSGYWNWIRKHPGKCLEYIGYISYNGITYHNPSLKSRITINRDTSKNQYSLQLNSVTPEDTAVYYCARYKYDYDGGHAMDYWGQGTLVTVSSGGGGSGGGGSGGGGSDIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGCGTKVEIK

SEQ ID NO: 154 - protein, 566 aa, Artificial Sequence
  7H11_hzG3v9_8 HC
QVQLVQSGAEVKKPGASVKVSCKASGYTFTRYYMHWVRQAPGQGLEWMGWIYPGEGSTKYAQKFQGRVTMTRDTSISTAYMELSRLRSDDTAVYYCAREYGNYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 155 - protein, 568 aa, Artificial Sequence
  11D4_hzG3v9 HC
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 156 - protein, 572 aa, Artificial Sequence
  106-222_hzG3v9 HC
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 157 - protein, 571 aa, Artificial Sequence
  9B12_hzG3v9 HC
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 158 - protein, 567 aa, Artificial Sequence
  2H6_hzG3v9_8 HC
EVQLVESGGGLVQPGGSLRLSCAVSGYSITSDYAWNWIRQAPGKCLEWMGYISYSGSTAYNPSLKSRITISRDTSKNTFYLQMNSLRAEDTAVYYCARGPYNYLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGTEVQLLESGGGEVQPGGSLRLSCAASGFTFSDAFMYWVRQAPGKEREWVSSISNRGLKTAYAESVKGRFTISRDNAKNTLYLQMSSLRAEDTAVYYCVEGDWNLGPRGQGTLVTVKP

SEQ ID NO: 159 - protein, 163 aa, Artificial Sequence
  hsOX40-CRD(29-168)-Avi-his
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDGLNDIFEAQKIEWHEHHHHHH

SEQ ID NO: 160 - protein, 148 aa, Artificial Sequence
  hsOX40-CRD(29-168)-His
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDSAHHHHHH

SEQ ID NO: 161 - protein, 497 aa, Artificial Sequence
  hsOX40-eGFP
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKIGGSATTSATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 162 - protein, 561 aa, Artificial Sequence
  2C10_3h56 HC
QVQLVQSGAEVKKPGASVKVSCKASGYTFTNYWMHWVRQAPGQRLEWIGYINPSNDYTKYNQKFKDRATLTADKSANTAYMELSSLRSEDTAVYYCARQGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGTEVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSS

SEQ ID NO: 163 - protein, 566 aa, Artificial Sequence
  ADC-1013_3h56 HC
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGTEVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSS

SEQ ID NO: 164 - protein, 566 aa, Artificial Sequence
  CD40.1_3h56 HC
QVQLVQSGAEVKKPGASVKVSCKASGYTFTGYNMNWVRQAPGQGLEWMGNIDPYYGNTNYNQKFKGRVTMTTDTSTSTAYMELRSLRSDDTAVYYCARLGLQLYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGTEVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSS

SEQ ID NO: 165 - protein, 573 aa, Artificial Sequence
  selicrelumab_3h56 HC
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGTEVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSS

SEQ ID NO: 166 - protein, 569 aa, Artificial Sequence
  teneleximab_3h56 HC
QIQLVQSGPELKKPGETVRISCKASGYAFTTTGMQWVQEMPGKGLKWIGWINTHSGVPKYVEDFKGRFAFSLETSANTAYLQISNLKNEDTATYFCVRSGNGNYDLAYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGTEVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSS

SEQ ID NO: 167 - protein, 213 aa, Artificial Sequence
  2C10 LC
EIVLTQSPATLSLSPGERATLSCSASSSVSYMHWYQQKPGQAPRRWIYDTSKLASGVPARFSGSGSGTDYTLTISSLEPEDFAVYYCHQLSSDPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 217 aa, Artificial Sequence
  ADC-1013 LC
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 169 - protein, 219 aa, Artificial Sequence
  CD40.1 LC
DIVMTQTPLSLPVTPGEPASISCRSSQSLVHSNANTYLHWYLQKPGQSPQLLIYKLTNRFFGVPDRFSGSGSGTDFTLKISRVEAEDVGVYYCSQSIHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 214 aa, Artificial Sequence
  Selicrelumab LC
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 214 aa, Artificial Sequence
  Teneleximab LC
DIVLTQSPATLSVTPGDRVSLSCRASQSISDYLHWYQQKSHESPRLLIKYASHSISGIPSRFSGSGSGSDFTLSINSVEPEDVGIYYCQHGHSFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 455 aa, Artificial Sequence
  selicrelumab IgG1 LALA HC
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 173 - protein, 448 aa, Artificial Sequence
  ADC-1013 IgG1 HC
EVQLLESGGGLVQPGGSLRLSCAASGFTFSTYGMHWVRQAPGKGLEWLSYISGGSSYIFYADSVRGRFTISRDNSENALYLQMNSLRAEDTAVYYCARILRGGSGMDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 348 aa, Artificial Sequence
  3h-56 IgG1 LALA HC
EVQLLESGGGLVQPGGSLRLSCAASGFTFRDYEMWWVRQAPGKGLERVSAINPQGTRTYYADSVMGRFTISRDNSKNTLYLQMNSLRAEDTAVYYCAKLPFTFDDWGQGTLVTVSSGGGGTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2020/0347143 A1
(43) Pub. Date: Nov. 5, 2020

(54) NOVEL TNFR AGONISTS AND USES THEREOF

(71) Applicant: Ichnos Sciences SA, La Chaux-de-Fonds (CH)

(72) Inventors: Stanislas BLEIN, La Chaux-de-Fonds (CH); Francois ROUSSEAU, La Chaux-de-Fonds (CH); Rami LISSILAA, La Chaux-de-Fonds (CH); Jonathan BACK, La Chaux-de-Fonds (CH); Julie MACOIN, La Chaux-de-Fonds (CH)

(73) Assignee: Glenmark Pharmaceuticals S.A., La Chaux-de-Fonds (CH)

(21) Appl. No.: 16/471,384

(22) PCT Filed: Dec. 19, 2017
(86) PCT No.: PCT/EP2017/083632

§ 371 Date: Jun. 19, 2019

(30) Foreign Application Priority Data
Dec. 19, 2016 (EP) 16205108.0

(51) Int. Cl. C07K 16/28

(52) U.S. Cl. CPC C07K 16/2878; C07K 2317/24; C07K 2317/92; C07K 2317/94; C07K 2317/75; C07K 2317/622; C07K 2317/33; C07K 2317/35; C07K 2317/567

(57) ABSTRACT

The present invention relates to a new class of TNFR agonist comprising multiple binding portions to two different parts of the same TNFR. The present invention also relates to methods of activating components of the immune system in a patient via the administration of a TNFR agonist according to the present invention as well as the use of such materials for further therapeutic and other purposes.

### REFERENCE TO SEQUENCE LISTING SUBMITTED ELECTRONICALLY

The content of the electronically submitted sequence listing (Name 3305_0260001_Seqlisting_st25; Size: 486,348 bytes; and Date of Creation: Jun. 12, 2019) is incorporated herein by reference in its entirety.

The present invention relates to a new class of Tumour Necrosis Factor Receptor Super Family (TNFR) agonists comprising multiple binding portions to at least two different portions of the TNFR. The present invention also relates to methods of activating components of the immune system in a patient via the administration of the TNFR agonist according to the present invention as well as the use of such materials for therapeutic and other purposes.

### INTRODUCTION

Immunotherapy has become a major focus of innovation in the development of anti-cancer therapies, as when successful patients have long-lasting anti-tumour immune responses that not only eradicate primary tumours but also metastatic lesions and can lead to the establishment of a protective anti-tumour memory immune response. Investigators have focused and had great success with therapies which offset checkpoint inhibitors, such as CTLA-4 and PD-1 that remove in vivo inhibition of anti-tumor T cell responses through antibody-mediated antagonism of these receptors. It is increasingly clear however that removing the effects of one or more checkpoint inhibitor is not sufficient to promote tumor regression in a majority of patients. Generating a robust therapeutic immune response requires not only removing inhibitory pathways but also activating stimulatory pathways.

Within a tumour the presence of checkpoint inhibitors, can inhibit T cell function to suppress anti-tumor immune responses. Checkpoint inhibitors, such as CTLA-4 and PD-1, attenuate T cell proliferation and cytokine production. CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors. When used as single agents, these drugs can induce potent clinical and immunologic responses in patients with metastatic disease. However, each of these agents only benefits a subset of patients, highlighting the critical need for more effective combinatorial therapeutic strategies acting via more pathways/components of the immune system.

The members of the tumour necrosis factor (TNF)/tumour necrosis factor receptor (TNFR) superfamily are critically involved in the maintenance of homeostasis in the immune system. The biological functions of the immune system encompass beneficial and protective effects in inflammation and host defence as well as a crucial role in organogenesis.

Members of the TNFR super family are listed in Table 1 below.

TABLE 1

| TNFR super | | | |
|---|---|---|---|
| family member | Synonyms | Gene | Ligand(s) |
| Tumor necrosis | CD120a | TNFRSF1A | TNF-alpha |
| factor receptor 1 | | | (cachectin) |
| Tumor necrosis | CD120b | TNFRSF1B | |
| factor receptor 2 | | | |
| Lymphotoxin | CD18 | LTBR | Lymphotoxin |
| beta receptor | | | beta (TNF-C) |
| OX40 | CD134 | TNFRSF4 | OX40L |
| CD40 | Bp50 | CD40 | CD154 |
| Fas receptor | Apo-1, CD95 | FAS | FasL |
| Decoy receptor 3 | TR6, M68 | TNFRSF6B | FasL, LIGHT, |
| | | | TL1A |
| CD27 | S152, Tp55 | CD27 | CD70, Siva |
| CD30 | Ki-1 | TNFRSF8 | CD153 |
| 4-1BB | CD137 | TNFRSF9 | 4-1BB ligand |
| Death receptor 4 | TRAILR1, Apo-2, | TNFRSF10A | TRAIL |
| | CD261 | | |
| Death receptor 5 | TRAILR2, CD262 | TNFRSF10B | |
| Decoy receptor 1 | TRAILR3, LIT, | TNFRSF10C | |
| | TRID, CD263 | | |
| Decoy receptor 2 | TRAILR4, TRUNDD, | TNFRSF10D | |
| | CD264 | | |
| RANK | CD265 | TNFRSF11A | RANKL |
| Osteoprotegerin | OCIF, TR1 | TNFRSF11B | |
| TWEAK receptor | Fnl4, CD266 | TNFRSF12A | TWEAK |
| TACI | IGAD2, CD267 | TNFRSF13B | APRIL, BAFF, |
| | | | CAMLG |
| BAFF receptor | CD268 | TNFRSF13C | BAFF |
| Herpesvirus entry | ATAR, TR2, CD270 | TNFRSF14 | LIGHT |
| mediator | | | |
| Nerve growth | p75NTR, CD271 | NGFR | NGF, BDNF, |
| factor receptor) | | | NT-3, NT-4 |
| B-cell maturation | TNFRSF13A, CD269 | TNFRSF17 | BAFF |
| antigen | | | |
| Glucocorticoid- | AITR, CD357 | TNFRSF18 | GITR ligand |
| induced | | | |
| TNFR-related | | | |
| TROY | TAJ, TRADE | TNFRSF19 | unknown |
| Death receptor 6 | CD358 | TNFRSF21 | |
| Death receptor 3 | Apo-3, TRAMP, | TNFRSF25 | TL1A |
| | LARD, WS-1 | | |
| Ectodysplasin A2 | XEDAR | EDA2R | EDA-A2 |
| receptor | | | |

OX40 (CD134; TNFRSF4) is a member of the TNFR super-family and was originally characterized as a receptor that was primarily expressed by rat CD4 T cells from the thymus and lymph nodes following stimulation with concanavalin A. Subsequent research demonstrated that in both mice and humans, OX40 is expressed by CD4 and CD8 T cells during antigen-specific priming and that OX40 expression is induced following TCR/CD3 cross-linking, and by the presence of inflammatory cytokines, including IL-1, IL-2, and TNF-α. The expression of OX40 following antigen encounter is largely transient for both CD4 and CD8 T cells (24-72 h), with the duration of OX40 expression by CD8 T cells reported to be shorter than for CD4 T cells. In the absence of activating signals, relatively few mature T cell subsets have been shown to express OX40 at biologically relevant levels. However, the constitutive expression of OX40 by follicular helper CD4 T cells (Tfh) has been described in both mice and humans. Within germinal centers, the CD4+/CXCR5+/CCR7− subpopulation of Tfh cells have been shown to have the highest level of OX40 expression and are thought to be important regulators of antibody production. In mice, OX40 is also constitutively expressed on FoxP3+ regulatory T cells (Treg cells), in contrast to human Treg cells where its expression is inducible. In contrast, antigen-specific activation can induce OX40 expression by numerous subsets of differentiated CD4 and CD8 T cells. In a murine model system (OT-II), Th1 and Th17 cells were both capable of a similarly robust induction of OX40 in response to peptide-activation. In humans, a substantial proportion of tumor-infiltrating CD4 T cells express OX40, presumably due to recognition of tumor antigens, and the frequency of OX40+CD4 T cells may be prognostic for patient outcomes. Similarly, activated peripheral CD8 T cells have also been shown to express OX40 in mice and humans.

Ligation of OX40 on CD8 and conventional (non-regulatory) CD4 T cells, using either its natural ligand (OX40L) or agonist antibodies, promotes their survival and expansion. Evidence of this comes from studies using OX40- and OX40L-deficient mice, which are discussed in detail in several recent reviews. These studies demonstrated that OX40- or OX40L-knockout mice had reduced expansion of both CD4 and CD8 T cells, combined with defective memory responses following antigen challenge, indicating the importance of endogenous OX40 expression in regulating T cell expansion. Furthermore, treatment with agonist anti-OX40 monoclonal antibodies (mAbs) along with TCR stimulation in wild-type animals induced expansion, differentiation, and increased survival of CD4 and CD8 T cells. Likewise, depletion of CD8 or CD4 T cells eliminated the ability of anti-OX40 mAbs to induce tumor regression in several tumor models. One study demonstrated that anti-OX40 administration was sufficient to overcome CD8 T cell tolerance to a self-antigen and restored their cytotoxic activity, highlighting the therapeutic potential for OX40 agonists. This is of particular importance for patients with cancer, as T cell tolerance to the tumor is a major obstacle for therapeutic modalities.

Another group has demonstrated that enhanced CD8 T cell function following anti-OX40 treatment was mediated by the induction of CD40L expression on effector T cells thereby promoting DC maturation, because CD40−/− mice have significantly fewer CD11c+ dendritic cells that migrate into the draining lymph nodes following anti-OX40 mAb. In fact, CD40−/− mice treated with anti-OX40 mAbs all succumb to their tumors in contrast to wild-type mice, which have a 60% survival rate, suggesting the importance of CD40 expression following OX40 stimulation. Collectively, these data suggest that exogenous manipulation of OX40 signaling can boost stagnant T cell responses. Several investigators have conducted studies to determine the mechanism by which OX40 promotes T cell survival. It has been demonstrated that following activation, OX40-deficient CD4 T cells failed to sustain expression of the anti-apoptotic proteins Bcl-xL and Bcl-2. Moreover, the survival of activated CD4 T cells was rescued by retroviral transduction of Bcl-xL or Bcl-2. Sustained expression of Bcl-xL was also necessary for the survival of tumor-reactive CD8 T cells following OX40 co-stimulation. Subsequent studies demonstrated that OX40 signaling in T cells induced expression of Survivin, and this was required to regulate and sustain T cell division over time. Survivin expression was maintained via the sustained activation of PI3K and PKB by OX40 signaling. However, Survivin expression does not supersede the requirement for Bcl-xL and Bcl-2 following OX40 signaling in order to inhibit T cell apoptosis. Enhanced expression of Survivin and Bcl-2 family members is mediated via activation of IκB kinase and NF-κB1 following OX40 signaling. Other investigators have shown that TRAF2 is required following OX40 signaling in antigen-specific CD4 T cells, as the expression of a dominant negative TRAF2 in CD4 T cells inhibited their expansion, survival, and cytokine production. One of the functions of TRAF2 appears to be to prevent CTLA-4 expression following T cell co-stimulation through OX40, as CTLA-4 blockade at the time of T cell priming with antigen and anti-OX40 mAbs partially restored defective expansion in mice expressing a dominant negative TRAF2 protein. It remains unknown whether the same TRAF adaptors and NF-κB pathways are activated in T cells following ligand binding by other TNFR family members, such as CD27 and GITR.

Similarities and differences in the signaling pathways activated by T cell co-stimulatory receptors, including both TNFR family members, like OX40 and CD27, and immunoglobulin super-family members, like CD28 and B7 families, has been reviewed extensively elsewhere. The activation of multiple pathways by both co-stimulatory receptor super-families results in enhanced cell growth and effector function, and improves survival. Numerous investigators are currently testing the modulation of these receptors for various clinical applications and immunotherapies. Preclinical studies demonstrated that treatment of tumorbearing hosts with OX40 agonists, including both anti-OX40 mAb and OX40L-Fc fusion proteins, resulted in tumor regression in several preclinical models. Recent studies have investigated the mechanisms by which these agonists function. In addition to promoting effector T cell expansion, since OX40 is constitutively expressed on Treg cells, OX40 agonists have the ability to directly regulate Treg cells. There are conflicting reports on whether these agonists promote or diminish Treg cell responses. Some have observed that anti-OX40 mAbs blocked the suppressive function of Treg cells in vivo, while others have observed Treg cell expansion. These studies suggest that anti-OX40 can push Treg cells in both directions, depending upon the context of stimulation and the cytokine milieu. Indeed, the importance of the OX40 co-stimulatory pathway in regulating immunity is exemplified by the presence of autoimmune-like disease in mice with constitutive expression of OX40L. OX40 signaling has also been shown to inhibit the production of IL-10 by and suppressive function of Treg cells. Supporting these data, administration of anti-OX40 mAbs prior to tumor engraftment rendered Treg cells functionally inactive through inhibition of IL-10 production and elimination of Treg cell-mediated suppression of CD8 T cell responses. One recent report observed that cells expressing activating FcγR were required for the selective depletion of Treg cells from tumors, while there was no change in Treg cells in the draining lymph nodes at day 5 following anti-OX40 therapy. Other studies confirm that even at later time points following anti-OX40 treatment, there is no change in the frequency of Treg cells in the draining lymph nodes, so this effect may be localized to the tumor. In fact, this effect may be transient, as another report showed that at day 7 there was no difference in Treg cell frequency in the tumor between control-treated and anti-OX40-treated mice using the same CT26 colon cancer model. This study in particular also suggests that the immunological effects of anti-OX40 therapy can vary based on the tumor model examined; thus, one must be cautious of making generalizations regarding the precise mechanism of OX40 agonists. Other studies report that anti-OX40 mAbs reduce the suppressive activity of Treg cells in vitro and in vivo. Whether anti-OX40 functions via Treg cell suppression, deletion, or both, treatment with these agonists should diminish the inhibitory effects mediated by Treg cells and thereby promote antitumor CD8 T cell responses necessary to maintain long-term antitumor immune responses. It is likely that multiple mechanisms are important for the anti-tumour activity of OX40 agonists.

Given the complexity and plasticity of the human immune system and the further complexity of dealing with the immune system of a cancer patient which is being purposively disrupted by tumour cells in order to evade eliciting a curative immune response, combination therapies modulating different immune system receptors/cell populations are increasingly being proposed and validated both preclinically and in patients. For instance workers in the field have shown that the sequencing of PD-1 antagonistic antibodies and OX40 agonistic antibodies is critical with concurrent administration leading to a negation of the effects of the OX40 agonist (Shrimali et al., Cancer Immunol Res; 5(9); 1-12) and Messenhiemer et., Clin Cancer Res. 2017 Oct. 15; 23(20):6165-6177).

This complexity also means that as the field of immune-oncology develops further and the understanding of the optimal ways to elicit a therapeutic immune response using immunomodulatory agents increases, it is going to be essential to generate pharmacologically active substances against as broad a range of relevant targets as possible, TNFRs represent perhaps the most important class of immuno-oncology target and the generation of pharmacologically active agonists has proven difficult to date.

### SUMMARY OF THE INVENTION

The present invention relates to TNFR agonists comprising binding portions to at least two different parts of a TNFR.

The inventors have surprisingly found that agonists comprising binding portions which bind to at least two different parts or epitopes of a TNFR show levels of agonism better than the effect of the binding portions when not comprised in the same agonist and in comparison to the native ligand of the TNFR and other previously known agonists of the TNFR.

In accordance with the present invention the TNFR is selected from the group shown in Table 1 or any other member of the TNFR superfamily.

Preferably the TNFR is involved in costimulation of T cell responses.

Preferably the TNFR is selected from the group comprising: CD27, 4-1BB (CD137), OX40 (CD134), HVEM, CD30, and GITR and most preferably is OX40.

In accordance with the present invention the term ‘two different parts of the TNF receptor’ shall mean two portions of the TNFR which can be simultaneously bound by the one of each of the binding portions, meaning that they can bind simultaneously on the same TNFR or bridge between two identical TNFRs by binding to these simultaneously.

In particular the present invention relates to binding portions from protein based target specific binding molecules such as antibodies, DARPins, Fynomers, Affimers, variable lymphocyte receptors, anticalin, nanofitin, variable new antigen receptor (VNAR), but is not limited to these.

In particular the TNFR comprises binding portions taken or derived from an antibody such as a Fab, Fab′, Fab′-SH, Fd, Fv, dAb, F(ab′)2, scFv, Fcabs, bispecific single chain Fv dimers, diabodies, triabodies. In preferred embodiments the agonist comprises binding portions taken or derived from Fab, ScFv and dAb.

In accordance with another aspect of the present invention the binding portions comprised with the agonist are of different types, a preferred embodiment combines Fab and scFv or Fab and dAb binding portions in the same agonist.

Method are known to transform Fab binding portions into other types of binding portions such as scFvs, dAbs, scFabs and similarly to transform such binding portions into Fabs interchangeably.

In particular the binding portions maybe genetically fused to a scaffold comprising the same or a different antibody Fc or a portion thereof. In accordance with this aspect of the present invention, a first full length antibody such as an IgG may form the basis of an agonist according of the present invention and a second set of binding portions may be grated onto the starting antibody in accordance with the present invention.

Alternatively the binding portions maybe genetically fused to a scaffold other than one derived from the Fc of an immunoglobulin, such as those based upon the SH3 domain of Fyn as used in fynomers and those based upon the human protease inhibitor Stefin A used in Affimers.

According to the present invention the binding portions which bind to different portions of the TNFR are disposed at the C and N terminus of the scaffold comprised within the TNFR agonist respectively.

In accordance with another aspect of the present invention the binding portions are disposed at either the C or N terminus and are concatenated.

Preferably the binding portions which bind to the same portion of the TNFR are disposed at the same terminus of the agonist. In accordance with the present invention, the binding portions to a first part of the TNFR are disposed at the C or N terminus and the binding portions to a second part of the TNFR are disposed at the opposite terminus. The inventors have found that the binding portions to the same part of the target TNFR should be preferentially disposed on the same terminus of the agonist.

In accordance with another aspect of the present invention the binding portion may be nucleotide based such as an aptamer.

Preferably the agonist comprises more than two binding portions.

More preferably the agonist comprises four or more binding portions.

Preferably the agonist comprises at least two binding portions that bind to the same part/epitope of the TNFR.

Most preferably the agonist comprises at least two sets of two identical binding portions. The inventors have found that TNFR agonists comprising two binding portions to each of the parts/epitopes of the TNFR and which are disposed at either end of the agonist show consistently high levels of agonism.

In particular the inventors have found that agonists which comprise binding portions that bind to different cysteine-rich domains (CRD) of the same TNFR, meaning that they comprise membrane proximal and membrane distal binding portions from different cysteine-rich domains (CRD) of the TNFR.

Preferably the agonist binds to a membrane proximal and membrane distal epitope.

In accordance with a further aspect of the present invention relates to an OX40 receptor (OX40) agonist which comprises multiple OX40 binding portions to two different parts/epitopes of OX40.

In accordance with the present invention the OX40 agonist binds to epitopes in cysteine-rich domain (CRD) 1 and CRD 3 of OX40. Alternatively the OX40 agonist binds to CRD 1 and CRD 4.

In accordance with a further aspect of the present invention the OX40 binding portion is selected from a sequence selected from the group comprising: SEQ ID NO: 2, 3, 12, 13, 14, 15, 16, 17, 18, 18, 19, 20, 21, 22, 23, 24, 25, 26, 27, 28, 29, 30, 31, 32, 35, 36, 37, 38, 39, 40, 41, 42, 43, 44, 45, 46, 47, 48, 49, or isolated polypeptides having an amino acid sequence that is at least 50%, 60%, 65%, 70%, 75%, 80%, 85%, 90%, 91%, 92%, 93%, 94%, 95%, 96%, 97%, 98%, or 99% identical thereto. The present invention also relates to a construct comprising any of the other OX40 binding portions comprised in the specification and sequence listing.

In accordance with a preferred embodiment of the present invention the OX40 agonist is encoded by SEQ ID Nos: 45 and 16 or isolated polypeptides having an amino acid sequence that is at least 50%, 60%, 65%, 70%, 75%, 80%, 85%, 90%, 91%, 92%, 93%, 94%, 95%, 96%, 97%, 98%, or 99% thereto.

The present invention also relates to methods of activating components of the immune system in a patient via the administration of the OX40 agonist according to the present invention.

The present invention also relates to the use of the OX40 agonist according to the present invention as a medicament.

The present invention also relates to the use of the OX40 agonist according to the present invention as a medicament for the treatment of cancer, an immunological disorder or other disease characterised or exasperated by under activation of the patient's immune system.

The present invention also relates to a method of treating a patient suffering from cancer, involving administering to the patient an effective amount of the OX40 agonist.

The present invention also relates to a method of treating a patient suffering from cancer, involving administering to the patient an effective amount of the OX40 agonist and one or more other agents, such as small molecule or biological medicines to further modulate the immune system of the patient. Examples of such agents include anti-PD-1 antibodies and antineoplastic small molecules such as multikinase inhibitors.

Further the present invention relates to the co-administration of the OX40 agonist according to the present invention and another medicament to a patient, wherein the other medicament has a synergistic or additive effect.

In accordance with a further aspect of the present invention relates to a CD40 receptor (CD40) agonist which comprises multiple CD40 binding portions.

Preferably the agonist comprises more than two binding portions.

More preferably the agonist comprises four binding portions

Preferably the agonist comprises at least two identical binding portions.

Preferably the agonist comprises at least two sets of two identical binding portions.

Alternatively the agonist comprises at four binding portions which bind to the same epitope.

The present invention also relates to methods of activating components of the immune system in a patient via the administration of the CD40 agonist according to the present invention.

Use of the CD40 agonist according to the present invention as a medicament.

In accordance with another aspect of the present invention the TNFR agonist comprises two monoclonal antibodies which recognise and bind to two different portions of the same TNFR and with can be coadministered to a patient in need thereof.

Further the present invention relates to the co-administration of the TNFR agonist according to the present invention and another medicament to a patient, wherein the other medicament has a synergistic or additive effect.

A non-exhaustive list of medicaments include T cell redirecting multispecific antibodies, checkpoint inhibitors, immunomodulatory agents.

The present invention also relates to the use of such materials for further therapeutic and other uses.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses (also known as isotypes) as well, such as IgG1, IgG2, and others. Furthermore, in humans, the light chain may be a kappa chain or a lambda chain.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as“complementarity-determining regions,” or“CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The single domain antibody (sdAb) fragments portions of the fusion proteins of the present disclosure are referred to interchangeably herein as targeting polypeptides herein.

As used herein, the term“epitope” includes any protein determinant capable of specific binding to/by an immunoglobulin or fragment thereof, or a T-cell receptor. The term“epitope” includes any protein determinant capable of specific binding to/by an immunoglobulin or T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 mM, for example, in some embodiments, ≤1 μM; e.g., ≤100 nM, ≤10 nM or ≤1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (Kd) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the“on rate constant” (kon) and the“off rate constant” (koff) can be determined by calculation of the concentrations and the actual rates of association and dissociation. (See Nature 361:186-87 (1993)). The ratio of koff/kon enables the cancellation of all parameters not related to affinity, and is equal to the dissociation constant Kd. (See, generally, Davies et al. (1990) Annual Rev Biochem 59:439-473). An antibody of the present disclosure is said to specifically bind to an antigen, when the equilibrium binding constant (Kd) is ≤1 mM, in some embodiments, ≤1 μM, ≤100 nM, ≤10 nM, or ≤100 pM to about 1 pM, as measured by assays such as radioligand binding assays, surface plasmon resonance (SPR), flow cytometry binding assay, or similar assays known to those skilled in the art.

The term“isolated protein” referred to herein means a protein of cDNA, recombinant RNA, or synthetic origin or some combination thereof, which by virtue of its origin, or source of derivation, the“isolated protein” (1) is not associated with proteins found in nature, (2) is free of other proteins from the same source, e.g., free of marine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, for example, at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland7 Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, for example, at least 90 percent sequence identity, at least 95 percent sequence identity, or at least 99 percent sequence identity.

In some embodiments, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Suitable conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present disclosure, providing that the variations in the amino acid sequence maintain at least 75%, for example, at least 80%, 90%, 95%, or 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Suitable amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (for example, conservative amino acid substitutions) may be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, for example, at least 14 amino acids long, at least 20 amino acids long, at least 50 amino acids long, or at least 70 amino acids long. The term “analog” as used herein refers to polypeptides which are comprised of a segment of at least 25 amino acids that has substantial identity to a portion of a deduced amino acid sequence and which has specific binding to CD47, under suitable binding conditions. Typically, polypeptide analogs comprise a conservative amino acid substitution (or addition or deletion) with respect to the naturally-occurring sequence. Analogs typically are at least 20 amino acids long, for example, at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TINS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH2NH—, —CH2S—, —CH2-CH2-, —CH═CH-(cis and trans), —COCH2-, CH(OH)CH2-, and —CH2SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, and/or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term“pharmaceutical agent or drug” as used herein refers to a chemical compound or composition capable of inducing a desired therapeutic effect when properly administered to a patient.

The term “antineoplastic agent” is used herein to refer to agents that have the functional property of inhibiting a development or progression of a neoplasm in a human, particularly a malignant (cancerous) lesion, such as a carcinoma, sarcoma, lymphoma, or leukemia. Inhibition of metastasis is frequently a property of antineoplastic agents.

As used herein, the terms “treat,” treating,” “treatment,” and the like refer to reducing and/or ameliorating a disorder and/or symptoms associated therewith. By “alleviate” and/or “alleviating” is meant decrease, suppress, attenuate, diminish, arrest, and/or stabilize the development or progression of a disease such as, for example, a cancer. It will be appreciated that, although not precluded, treating a disorder or condition does not require that the disorder, condition or symptoms associated therewith be completely eliminated.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, for example, more than about 85%, 90%, 95%, and 99%. In some embodiments, the object species is purified to essential homogeneity (contaminant species cannot be detected in the composition by conventional detection methods) wherein the composition consists essentially of a single macromolecular species.

In this disclosure, “comprises,” “comprising,” “containing,” “having,” and the like can have the meaning ascribed to them in U.S. and/or European Patent law and can mean “includes,” “including,” and the like; the terms “consisting essentially of” or “consists essentially” likewise have the meaning ascribed in U.S. patent law and these terms are open-ended, allowing for the presence of more than that which is recited so long as basic or novel characteristics of that which is recited are not changed by the presence of more than that which is recited, but excludes prior art embodiments.

By “effective amount” is meant the amount required to ameliorate the symptoms of a disease relative to an untreated patient. The effective amount of active compound(s) used to practice the present invention for therapeutic treatment of a disease varies depending upon the manner of administration, the age, body weight, and general health of the subject.

Ultimately, the attending physician or veterinarian will decide the appropriate amount and dosage regimen. Such amount is referred to as an “effective” amount.

By “subject” is meant a mammal, including, but not limited to, a human or non-human mammal, such as a bovine, equine, canine, rodent, ovine, primate, camelid, or feline.

The term “administering,” as used herein, refers to any mode of transferring, delivering, introducing, or transporting a therapeutic agent to a subject in need of treatment with such an agent. Such modes include, but are not limited to, oral, topical, intravenous, intraperitoneal, intramuscular, intradermal, intranasal, and subcutaneous administration.

### BRIEF DESCRIPTION OF THE FIGURES

FIG. 1: SPR sensorgram of the binding of 2H6 scFv-Fc (upper line) and 2H6 M108L scFv-Fc (lower line) at fixed concentration (200 nM) to human OX40R captured on CM5 chip with a ligand density of 600 RU at 25° C.

FIG. 2: The graph shows the results of normalized 3H-thymidine incorporation from 4 independent MLR experiments with the mean±SD. Each data point is the mean of triplicate values of an individual allogeneic combinations. The dotted line represents the level of the allogeneic reaction (No antibody). All the combinations were not significantly different (ns).

FIG. 3: PBMCs were incubated in the presence of the SEB with or without antibodies for 7 days; supernatants were harvested on day 5. The graphs show the mean±SD of normalized absolute counts of CD4 CD25+ per well (A) and normalized IL-2 concentration (B) from 5 independent experiments. Each data point is the mean of triplicate values and represents an independent PBMC donor. The dotted lines represent the level of the condition in which PBMCs were incubated only with SEB (No antibody). ns, not significant; *, p<0.05; ***, p<0.001 were obtained using the one-tailed non-parametric Mann-Whitney test.

FIG. 4: PBMCs were incubated in presence of PHA with or without antibodies for 5 days. The graph shows the results of normalized 3H-thymidine incorporation from 3 independent experiments with the mean±SD. Each data point is the mean of triplicate values and represents an independent PBMC donor. The dotted line represents the level of the condition in which PBMCs were incubated only with PHA (No antibody). ns stands for not significant.

FIG. 5: SDS-PAGE analysis of Tetra-1 and Tetra-8. A photograph of a Coomassie blue stain SDS-PAGE gel under non-reducing conditions of Tetra-1 and Tetra-8 obtained after protein A purification. (MW) molecular weight markers as indicated.

FIG. 6: Analytical size exclusion chromatography of Tetra-1 and Tetra-8. FIGS. 6A and 6B are a series of graphs depicting the elution profile from a size exclusion chromatography (SEC) column for Tetra-1 (FIG. 6A) and Tetra-8 (FIG. 6B). The peak area percentage (%) which indicates the % of the total ‘detectable’ peaks in the sample chromatogram (taken as 100%) was calculated for each peaks depending on their retention time and indicated in tables for Tetra-1 (FIG. 6C) and Tetra-8 (FIG. 6D).

FIG. 7: Cation exchange purification of Tetra-8. FIG. 7A shows a graph depicting the elution profile of Tetra-8 (dotted line) from a cation exchange HiTrap SP HP column. The sodium acetate gradient used for protein separation is indicated by a black line. FIG. 7B is a photograph of a Coomassie blue stain SDS-PAGE gel under non-reducing conditions of the different fractions collected from the cation exchange purification chromatography of Tetra-8.

FIG. 8: Thermal stability assessment of Tetra-1 and Tetra-8 by Differential Scanning calorimetry. FIGS. 8A and 8B are graphs representing thermo-stability measurements of Tetra-1 and Tetra-8, respectively, using differential scanning calorimetry (DSC). Data are expressed as excess molar heat capacity (abbreviated Cp [kcal/mol/° C.]; Y axis) vs. temperature (° C.; X axis). Unfolding events corresponding to the scFv, Fab, CH2 and CH3 domains are indicated.

FIG. 9: Structure of the extracellular domain of OX40. Ribbon representation of the extracellular domain of human OX40 (RCSB: 2HEV). The cysteine-rich domains (CRD) are highlighted using grey or black colors, alternatively. Disulfide bonds are depicted by spheres.

FIG. 10: Alignment of human, cynomolgus monkey and rat OX40 extracellular domains. Multiple sequence alignment of human (SEQ ID NO: 1), cynomolgus monkey (SEQ ID NO: 122) (abbreviated cyno) and rat OX40 (SEQ ID NO: 121) extracellular domains prepared with T-coffee. CRDs are indicated by boxes of white or black colors. Disulfide bond pairings are indicate by arrows. Residues which are strictly conserved between species are shaded in black, residues with 70% conservation are shaded in grey.

FIG. 11. A dose-response of various antibodies was incubated on recombinant Human OX40 receptor, then detected with anti-human Fab fragment specific coupled with Horseradish Peroxidase enzyme. The graphs show the nonlinear sigmoidal regression binding curves (Absorbance at 450 nM) for each treatment. The following treatments were tested: Tetra-8 (◯), 7H11_v8 IgG1 (□), Tetra-22 (∇). Each data point is the mean±SD of duplicate values.

FIG. 12. A dose-response of various antibodies was incubated on JURKAT-NFkB-OX40 cells, then detected with an anti-human Fc fragment specific coupled with Phycoerythrin. The graph shows the nonlinear sigmoidal regression binding curves (Geometric Mean of Intensity) for each treatment. The following treatments were tested: Tetra-8 (◯), 7H11_v8 IgG1 (□), 2H6 IgG1 (Δ), Control IgG (∇).

FIG. 13. A dose-response of various antibodies was incubated on various receptors, members of Tumor Necrosis Factor Receptor family, then detected with Streptavidin coupled with Horseradish Peroxidase enzyme. The same treatments were tested on all receptors: Tetra-8 (◯), 7H11 IgG1 (□), 2H6 IgG1 (Δ), Control IgG (∇), respective commercial positive control (X). The graphs show the nonlinear sigmoidal regression binding curves (Absorbance at 450 nM) for each treatment. Each data point is the mean of duplicate values except for control curves that were performed in simplicate.

FIG. 14. A dose-response of antibodies was incubated on recombinant cynomolgus OX40, then detected with anti-Human Fab fragment specific coupled with Horseradish Peroxidase enzyme. The graphs show the nonlinear sigmoidal regression binding curves (Absorbance at 450 nM) for each condition. The following treatments were tested: Tetra-8 (◯), 7H11 IgG1 (□), Tetra-22(Δ). Each data point is the mean±SD of duplicate values.

FIG. 15. Antibodies were incubated on Human and Cynomolgous PBMC, then detected with anti-Human Fc fragment specific coupled with Phycoerythrin. The graphs represent an overlay of multiple histograms (Geometric Mean of Fluorescence) for each antibody on either Human or Rhesus CD4+ T cells.

FIG. 16. JURKAT-NFkB-OX40 cells were transferred to OKT3 pre-coated (5 μg/mL; overnight) or regular luminescence plates. Subsequently, a dose-response of antibodies or controls was incubated on JURKAT-NFkB-OX40 cells. After 5 h of incubation, Luciferase substrate was added to the wells and luminescence was measured using a microplate reader (read tape—endpoint; integration time—1 minute; emission—hole; optics position—top; gain 135; read height—1.00 mm). The graph shows the nonlinear sigmoidal regression binding curves (Luminescence) for each condition. The following treatments were tested: Tetra-8 (◯), 7H11 IgG1 LALA (□), 2H6 IgG1 LALA (Δ), Control IgG (∇), OX40L (X). Each data point is the mean±SD of duplicate values.

FIG. 17. Dendritic cells (DC) were differentiated for 6 days then co-cultured with freshly isolated CD4+ T cells. Dose-response of antibodies or controls were incubated on such cells, then after 6 days of incubation, tritiated-thymidine was added for 18 to 20 additional hours of incubation. Proliferation Index was calculated with the following method: thymidine incorporation background induced in autologous condition (CD4+ T cells only) was subtracted for each sample (specific for each CD4+ T cell donor), then this results was divided by the thymidine incorporation induced in allogeneic condition. The graphs show the proliferation index for each treatment. Each data point is the mean of triplicate values obtained for each DC-CD4+ T cells combination. N=36 combinations. The line Y=1 represents the normalized allogenic response.

FIG. 18. PBMC were isolated from filters and incubated with Staphylococcal enterotoxin B superantigen (SEB) in the presence of antibodies or controls. After 5 days of incubation, supernatants were harvested and quantified on Luminex for IL-2 release. Normalized IL-2 release was calculated with the following method: IL-2 quantification induced in non-stimulated cells (PBMC without SEB) was subtracted for each sample (specific for each PBMC donor), then this results was divided by the IL-2 release induced in SEB-stimulated cells (No treatment). Filled heavy line represents the response threshold. The graphs show the normalized IL-2 release for each treatment. Each data point is the mean of triplicate values obtained for each PBMC donor. N=17 PBMC donors. The line Y=1 represents the normalized SEB only induced-response.

FIG. 19. PBMC were isolated from filters and incubated with Staphylococcal enterotoxin B superantigen (SEB) in the presence of antibodies or controls at 80 and 10 nM. After 5 days of incubation, supernatants were harvested and quantified on Luminex for IL-2 release. Normalized IL-2 release was calculated with the following method: IL-2 quantification induced in non-stimulated cells (PBMC without SEB) was subtracted for each sample (specific for each PBMC donor), then this results was divided by the IL-2 release induced in SEB-stimulated cells (No treatment). Filled heavy line represents the response threshold. The graphs show the normalized IL-2 release for each treatment. Each data point is the mean of triplicate values obtained for each PBMC donor. The line Y=1 represents the normalized SEB only induced-response.

FIG. 20: Schematic representation of molecules based on 7H11 and 2H6 binding units having different valences and architectures.

FIG. 21: Analysis of 7H11 and 2H6 binding to OX40 when fused in C-terminus as Fab or scFv format. Surface Plasmon Resonance (SPR) measurements of proteolytically cleaved tetravalent molecules near their hinge regions (the Fc-2H6 Fab/2H6 Fab, Fc-2H6 Fab/7H11 scFv, Fc-7H11 Fab/7H11 Fab and Fc-7H11 Fab/2H6 scFv, as indicated) for the chimeric OX40 molecules chiOX40R-Fc HHRH (FIG. 21A) or chiOX40R-Fc RRHH (FIG. 21B). Data are expressed as number of response units (abbreviated RU; Y axis) vs. time (X axis). FIG. 21C shows a schematic representation of the agonists used in the analysis.

FIG. 22: Determination of OX40 co-engagement by 7H11 Fab and 2H6 scFv when fused in C-terminus. Co-engagement measurements by SPR of the Fc-7H11 Fab/2H6 scFv fragment with chimeric OX40 molecules chiOX40R-Fc HHRH (FIG. 22A) or chiOX40R-Fc RRHH (FIG. 22B) immobilized on the CHIP and human OX40 (HHHH), chiOX40R-Fc (HHRH) and chiOX40R-Fc (RRHH) sequentially injected. Data are expressed as number of response units (abbreviated RU; Y axis) vs. time (X axis). FIG. 22C shows a schematic representation of the agonists used in the analysis.

FIG. 23: Determination of OX40 co-engagement by 7H11 scFv and 2H6 Fab when fused in C-terminus. Co-engagement measurements by SPR of the Fc-2H6 Fab/7H11 scFv fragment with chimeric OX40 molecules chiOX40R-Fc HHRH (FIG. 23A) or chiOX40R-Fc RRHH (FIG. 23B) immobilized on the CHIP and human OX40 (HHHH), chiOX40R-Fc (HHRH) and chiOX40R-Fc (RRHH) sequentially injected. Data are expressed as number of response units (abbreviated RU; Y axis) vs. time (X axis). FIG. 23C shows a schematic representation of the agonists used in the analysis.

FIG. 24: PBMC were isolated from filters and incubated with Staphylococcal enterotoxin B superantigen (SEB) in the presence of antibodies or controls at 80 and 10 nM. After 5 days of incubation, supernatants were harvested and quantified on Luminex for IL-2 release. Normalized IL-2 release was calculated with the following method: IL-2 quantification induced in non-stimulated cells (PBMC without SEB) was subtracted for each sample (specific for each PBMC donor), then this results was divided by the IL-2 release induced in SEB-stimulated cells (No treatment). Filled heavy line represents the response threshold. The graphs show the normalized IL-2 release for each treatment. Each data point is the mean of triplicate values obtained for each PBMC donor. The line Y=1 represents the normalized SEB only induced-response.

FIG. 25: PBMC were isolated from filters and incubated with Staphylococcal enterotoxin B superantigen (SEB) in the presence of antibodies or controls at 80 and 10 nM. After 5 days of incubation, supernatants were harvested and quantified on Luminex for IL-2 release. Normalized IL-2 release was calculated with the following method: IL-2 quantification induced in non-stimulated cells (PBMC without SEB) was subtracted for each sample (specific for each PBMC donor), then this results was divided by the IL-2 release induced in SEB-stimulated cells (No treatment). Filled heavy line represents the response threshold. The graphs show the normalized IL-2 release for each treatment. Each data point is the mean of triplicate values obtained for each PBMC donor. The line Y=1 represents the normalized SEB only induced-response.

FIG. 26: Overlay of analytical gel filtration chromatograms. Chromatograms for Tetra-8 alone, hOX40 alone and antibody-hOX40 complexes at 1:4 ratio were overlaid. The arrows indicating expected molecular weights correspond to the peaks of the calibration run and are Ferritin (440 kDa), Aldolase (158 kDa) and Carbonic anhydrase (29 kDa). Note the differences between Tetra-8 and reversed Tetra-8 (indicated by arrows)—Tetra-8 has a shoulder in V0 and the second peak is shifted to higher molecular weight compared to that of reversed Tetra-8.

FIG. 27: Tetra-8-hOX40 crystalline-like lattice. One possibility of a large, 2-dimensional lattice structure is shown. Two hOX40 per TETRA-8 were used to build an, in theory, infinitively large structure.

FIG. 28. Time lapse of OX40-GFP on Jurkat OX40-GFP cell line following treatment with Tetra-8. Jurkat expressing OX40 eGFP cells were incubated overnight at 37° C. and 5% CO2 on Fluorodish (WPI) cell culture dishes (20000cells/cm2) pre-coated with fibronectin (1 μg/cm2 in PBS). Tetra-8 was then added to the cell medium at 80 nM final concentration for various time intervals (ranging from 2.5 to 27.5 min) and cells were imaged using a Zeiss Inverted microscope Z1 equipped with a confocal module LSM 800 at 63× magnification.

FIG. 29. Confocal images of OX40 clusters induced by Tetra-8 and other OX40-targeting molecules. Jurkat OX40-GFP cells were treated for either 5, 10, or 20 minutes with various molecules targeting OX40 (Tetra-8, 1A7, OX40L and Tetra-14), used at either at 20 nM (A) or 80 nM (B).

FIG. 30. Quantitative analysis of OX40 clustering induced by various anti-OX40 molecules on Jurkat-OX40 GFP cell line. Confocal images of OX40 clusters induced by Tetra-8 and other OX40-targeting molecules on Jurkat OX40-GFP cells were analyzed using the Kurtosis method, as described in the example.

FIG. 31. DC activation assay. Dendritic cells (DC) were isolated from PBMC (3 donors from filters and one donor from whole blood) and differentiated for 6 days then cultured for two additional days in the presence of antibodies or controls. After incubation, cells were harvested and stained with anti-CD1c-APC, anti-CD80-PE, anti-CD86-PerCP-eF710 for Panel 1 or anti-CD1c-APC, anti-CD83-FITC, anti-HLA-DR-PerCP5.5 for Panel 2. The graph shows the percentage of overexpressing cells for CD83 and CD86 markers, compared to No treated DC, that are also expressing some of these markers constitutively. Each data point is the value for one DC donor. N=4 donors.

FIG. 32. A dose-response of antibodies or controls were incubated on thaw-and-use NFkB-Luc2P/U2OS cells. After 4 h of incubation, luciferase substrate was added to the wells and luminescence was measured using a microplate reader (read tape—endpoint; integration time—1 minute; emission—hole; optics position—top; gain 135; read height—1.00 mm). The graph shows the nonlinear sigmoidal regression binding curves (Luminescence) for each condition. The following treatments were tested: Selicrelumab IgG (◯), ADC-1013 IgG1 (□), 3h56 IgG1 LALA (Δ), Selicrelumab_3 h56 (●), ADC-1013_3 h56 (▪), CD40L (X). Each data point is the mean±SD of duplicate values.

### EXAMPLE 1

### Generation and Screening of Mouse Anti-Human OX40 Antibodies

To produce the recombinant human OX40-his protein, the extracellular region (amino acids 1-214 as set forth in SEQ ID NO: 1) of human TNFRSF4 was amplified by PCR adding a 3′ GSG-6×His linker and restriction sites for cloning. The PCR product was subsequently cloned in the modified pcDNA3.1(−) plasmid described above. This recombinant plasmid allowed for the expression of the human OX40-his protein in mammalian cells with secretion into the cell culture media driven by the native signal peptide of the human TNFRSF4. For protein production, the recombinant vector was transfected into suspension-adapted HEK 293 cells (ATCC number CRL 1573) using jetPEI™ transfection reagent (Polyplus-transfection S.A., Strasbourg, France; distributor: Brunschwig, Basel, Switzerland). The cell culture supernatant was collected five days after transfection and purified using a Ni²⁺-NTA affinity purification column (HiTrap Ni²⁺-NTA sepharose column; GE Healthcare Europe GmbH, Glattbrugg, Switzerland) operated on an ÄKTA FPLC system (GE Healthcare Europe GmbH, Glattbrugg, Switzerland).

Recombinant human OX40-Fc and OX40-his proteins were found to be 95% pure as judged by SDS-PAGE, and further buffered exchanged into phosphate buffer saline (PBS) prior use.

To produce the recombinant human OX40L-Fc protein, a cDNA for the human TNFSF4 was purchased from imaGenes (clone name: IOH46203, Berlin, Germany) and the extracellular portion (amino acids 51-183) of human TNFSF4 ligand (numbering according to the Uniprot Q6FGS4 sequence) was amplified with flanking restriction sites for subsequent cloning into a modified mammalian expression vector based on the pcDNA3.1(−) plasmid from Invitrogen (Invitrogen AG, Basel, Switzerland, Cat. No. V795-20), containing the human Fc region of a human IgG1 (EU positions 223-451), the human CMV promoter with the Ig donor acceptor fragment (first intron) described in U.S. Pat. No. 5,924,939, the OriP sequence (Koons et al. 2001, J Virol. 75 (22):10582-92.), the SV40 enhancer, and the SV40 polyA fused to the gastrin terminator as described by Kim et al. (2003, Biotechnol Prog. 19 (5), p. 1620-2). This recombinant plasmid allowed for expression of the human TNFSF4 extracellular domain-Fc fusion protein in mammalian cells with secretion into the cell culture medium driven by the VJ2C leader peptide. For recombinant protein production, the aforementioned recombinant vector was transfected into suspension-adapted HEK 293 cells (ATCC number CRL 1573) using cationic polymers. The cell culture supernatant was collected after five days and further purified in batch using CaptivA™ primAB affinity beads (Repligen, Waltham, Mass., USA) and further buffer-exchanged to phosphate buffer saline (PBS) prior to use.

To produce the recombinant macaca OX40-Fc protein, a synthetic gene corresponding to the extracellular portion of macaca OX40 (amino acids 29-214 of NCBI sequence XP_001090870.1) was generated (GeneArt, ThermoFisher Scientific, Waltham, Mass.) with restriction sites for subsequent cloning into a modified mammalian expression vector based on the pcDNA3.1(−) plasmid from Invitrogen (Invitrogen AG, Basel, Switzerland, Cat. No. V795-20), containing the human Fc region of a human IgG1 (EU positions 223-451), the human CMV promoter with the Ig donor acceptor fragment (first intron) described in U.S. Pat. No. 5,924,939, the OriP sequence (Koons et al. 2001, J Virol. 75 (22):10582-92.), the SV40 enhancer, and the SV40 polyA fused to the gastrin terminator as described by Kim et al. (2003, Biotechnol Prog. 19 (5), p. 1620-2). This recombinant plasmid allowed for expression of the macaca OX40 extracellular domain-Fc fusion protein in mammalian cells with secretion into the cell culture medium driven by the VJ2C leader peptide. For recombinant protein production, the aforementioned recombinant vector was transfected into suspension-adapted HEK 293 cells (ATCC number CRL 1573) using cationic polymers. The cell culture supernatant was collected after five days and further purified in batch using CaptivA™ primAB affinity beads (Repligen, Waltham, Mass., USA) and further buffer-exchanged to phosphate buffer saline (PBS) prior to use. To produce the recombinant human OX40-Fc protein, a cDNA for the human TNFRSF4 was purchased from imaGenes (clone number: RZPDB737H0329D; Berlin, Germany). This cDNA was used as a template to PCR-amplify the DNA coding region of the human TNFRSF4 extracellular domain (amino acids 1-214 as set forth in SEQ ID NO: 1). In a separate PCR reaction, the Fc region of a human IgG1 (EU positions 223-451) was amplified. The two resulting products were then fused using overlap extension PCR with flanking primers, adding restriction sites for subsequent cloning into a modified mammalian expression vector based on the pcDNA3.1(−) plasmid from Invitrogen (Invitrogen AG, Basel, Switzerland, Cat. No. V795-20), containing the human CMV promoter with the Ig donor acceptor fragment (first intron) described in U.S. Pat. No. 5,924,939, the OriP sequence (Koons et al. 2001, J Virol. 75 (22):10582-92.), the SV40 enhancer, and the SV40 polyA fused to the gastrin terminator as described by Kim et al. (2003, Biotechnol Prog. 19 (5), p. 1620-2). This recombinant plasmid allowed for expression of the human TNFRSF4 extracellular domain-Fc fusion protein in mammalian cells with secretion into the cell culture medium driven by the native signal peptide of the human TNFRSF4 protein. For recombinant protein production, the aforementioned recombinant vector was transfected into suspension-adapted HEK 293 cells (ATCC number CRL 1573) using jetPEI™ transfection reagent (Polyplus-transfection S.A., Strasbourg, France; distributor: Brunschwig, Basel, Switzerland). The cell culture supernatant was collected after five days and further purified using a Protein A affinity purification column (HiTrap Protein A sepharose column; GE Healthcare Europe GmbH, Glattbrugg, Switzerland) operated on an ÄKTA FPLC system (GE Healthcare Europe GmbH, Glattbrugg, Switzerland).

Recombinant human OX40-Fc protein dissolved in PBS was mixed with an equal volume of Stimune adjuvant (Prionics, Switzerland, ref: 7925000) and an emulsion was prepared. The emulsion was transferred to 0.5 mL insulin syringes (BD Pharmingen, Allschwil, Switzerland) and BALB/c animals (Harlan, Netherlands) were immunized sub-cutaneously in the back footpads, the base of the tail and the neck with 50 μg of the emulsified protein. The immunization was repeated two weeks later with the same amount of antigen and the same route of injection.

The presence of circulating anti-human OX40 antibodies in the immunized mouse sera was evaluated by direct ELISA using plates coated with the recombinant human OX40-his protein.

A serial dilution (from 1:10⁰ to 1:10⁹) of the different mouse sera was added to the plates and the bound antibodies were detected using a goat anti-mouse H+L whole molecule-HRP (Sigma-Aldrich Chemie GmbH, Buchs, Switzerland).

A final sub-cutaneous boost with 50 μg of antigen without adjuvant was performed in animals displaying the best anti-human OX40 IgG serum titer 3 days before sacrifice.

Animals were euthanized and the inguinal, axillary, brachial, popliteal and sciatic lymph nodes were collected to prepare a single cell suspension by disturbing the lymph node architecture with two 25 G needles in a DNAse (Roche Diagnostics (Schweiz) AG, Rotkreuz, Switzerland) and collagenase (Roche Diagnostics (Schweiz) AG, Rotkreuz, Switzerland) solution. Single cell suspensions were fused to a myeloma cell line X63AG8.653 (mouse BALB/c myeloma cell line; ATCC accession number: CRL 1580; J Immunol 1979, 123:1548-1550)) at a ratio of 7:1 with polyethylene glycol 1500 (Roche Diagnostics (Schweiz) AG, Rotkreuz, Switzerland). The fused cells were plated into 96 well flat bottom plates containing mouse macrophages in DMEM-10 medium (Invitrogen AG, Basel, Switzerland) supplemented with 10% fetal bovine serum (FBS, PAA Laboratories, Pasching, Austria), 2 mM L-glutamine, 100 U/ml (Biochrom AG, Germany) penicillin, 100 μg/ml streptomycin (Biochrom AG, Germany), 10 mM HEPES (Invitrogen AG, Basel, Switzerland), 50 μM β-mercaptoethanol (Sigma-Aldrich Chemie GmbH, Buchs, Switzerland), HAT (Sigma-Aldrich Chemie GmbH, Buchs, Switzerland) and 1% Growth factor (Hybridokine, Interchim/Uptima, Montluçon, France).

Approximatively 800 hundred wells from the fusions were screened by ELISA for the presence of mouse IgG that recognized human OX40. Positive wells were expanded and subjected to two rounds of subcloning. Cells were collected and the heavy and light chains were cloned and sequenced.

### EXAMPLE 2

Cloning and Sequencing of the VH and VL Chains of the Anti-OX40 Antibodies from Hybridoma Cells

For each positively selected hybridoma, total RNA was prepared, reverse-transcribed into cDNA and VH and VL genes were respectively amplified by PCR. These PCR products were ligated into a rescue-vector (pDrive vector; QIAGEN AG, Hombrechtikon, Switzerland; Cat. No. 231124), allowing for the DNA sequencing of individual PCR products and the determination of mono- or poly-clonality of the selected hybridomas. This vector allowed for blue/white selection on LB-agar plates containing IPTG and X-gal (colonies with no insert were blue because of the degradation of X-gal by the LacZ α-peptide). Recombinant plasmids from positive (white) bacterial clones were prepared and sequenced using standard DNA sequencing primers specific for the vector backbone (M13rev, M13fwd, T7 or SP6). DNA sequences were finally subcloned into an expression vector for recombinant expression of the antibody of interest in mammalian cells.

### RNA Isolation

Total RNA was isolated from 2-10×10⁶ cells using the RNeasy Mini Kit from QIAGEN (QIAGEN AG, Hombrechtikon, Switzerland; Cat. No. 74106) according to the manufacturer's protocol; samples were quantified using a NanoDrop ND-1000 spectrophotometer (WITEC AG, Littau, Switzerland).

### One Step RT-PCR

The total RNA preparations described above were further reverse-transcribed into cDNA, and the VH and VL fragments were amplified by PCR using two different mixtures of degenerated primers, each one allowing the recovery of all the different subfamilies of mouse immunoglobulin heavy chain variable fragments and variable heavy chain junction regions or the recovery of all mouse immunoglobulin light chain kappa variable fragments and variable light chain kappa junction regions. The primers used for reverse transcription and amplification were synthesized by Microsynth (Balgach, Switzerland), and were HPLC purified (Tables 1-4). Both reverse-transcription and PCR amplification were performed simultaneously using the QIAGEN one step RT-PCR kit (QIAGEN AG, Hombrechtikon, Switzerland; Cat. No. 210212). Since the technique used specific primers, each mRNA sample was then treated in duplicate allowing for the individual reverse-transcription and amplification of either the VH or the VL fragments. 2μ.g of total RNA dissolved into RNase-free water to a final volume of 30 μl were mixed with: 10 μl of a 5× stock solution of QIAGEN OneStep RT-PCR Buffer, 2 μl of a dNTPs mix at a concentration of 10 mM, 3 μl of primer mix at a concentration of 10 μM and 2 μl of QIAGEN OneStep RT-PCR Enzyme Mix. The final mixture was then placed in a PCR tube, and cycled in a PCR-themocycler (BioRad iCycler version 4.006, Bio-rad Laboratories AG, Reinach, Switzerland) using the following settings:

30 min at 50° C.

15 min at 95° C.

40 cycles: 30 sec at 94° C.
30 sec at 55° C.
1 min at 72° C.

10 min at 72° C.

Hold at 4° C.

pDrive Cloning

PCR products were run onto 2% agarose gels. Following DNA electrophoresis, the fragments of interest (˜450 bp) were excised from the agarose gels, and further extracted using the Macherey-Nagel NucloSpin Extract II kit 250 (Macherey-Nagel, Oensingen, Switzerland; Cat. No. 740609.250). For DNA sequencing, the extracted PCR products were cloned into the rescue-vector described above (pDrive vector, QIAGEN AG, Hombrechtikon, Switzerland; Cat. No. 231124) and transformed into the E. coli TOP10 strain (Invitrogen AG, Basel, Switzerland; Cat. No. C404006)

### Miniprep Extraction

Positive colonies were cultured overnight at 37° C. (shaking 250 RPM) in 1.5 ml of Luria Bertani (LB) medium supplemented with 100 μg/ml ampicillin seeded in Macherey-Nagel Square-well Block plates (Macherey-Nagel, Oensingen, Switzerland; Cat. No. 740488.24). The next day DNA miniprep extractions were performed using the NucleoSpin Multi-8 Plasmid kit (Macherey-Nagel, Oensingen, Switzerland; Cat. No. 740620.5).

### Sequencing

Samples were sent for DNA sequencing to the DNA sequencing service company Fasteris (Plan-les-Ouates, Switzerland). The standard primers: M13rev, M13fwd, T7, SP6 were used (Table 5).

### Sequence Analysis

The Clone Manager 9 Professional Edition (Scientific & Educational Software, NC, USA) and the BioEdit Sequence Alignment Editor (Hall, T. A. 1999. BioEdit: a user-friendly biological sequence alignment editor and analysis program for Windows 95/98/NT. Nucl. Acids. Symp. Ser. 41:95-98) were used for the analysis of DNA sequences.

### Cloning of Expression Vector for Recombinant Chimeric Antibody Expression

For recombinant expression in mammalian cells, the isolated murine VH and VL fragments were formatted as chimeric immunoglobulins using assembly-based PCR methods. These chimeric antibodies consist of a heavy chain where the murine heavy chain variable domain is fused to the human IgG1 heavy chain constant domains (γ1, hinge, γ2, and γ3 regions) and a light chain where the murine light chain variable domain is fused to a human kappa constant domain (Cκ). PCR-assembled murine variable and human constant parts were subsequently cloned into a modified mammalian expression vector based on the modified pcDNA3.1(−) vector from Invitrogen mentioned in Example 1 with the difference that a human immunoglobulin light chain kappa leader peptide was employed to drive protein secretion. For protein production of the immunoglobulin candidates, equal quantities of heavy and light chain vector DNA were co-transfected into suspension-adapted HEK-293 (ATCC number: CRL-1573). The cell culture supernatant was collected after five days and purified using a Protein A affinity purification column (HiTrap Protein A sepharose column; GE Healthcare Europe GmbH, Glattbrugg, Switzerland) operated on an ÄKTA FPLC system (GE Healthcare Europe GmbH, Glattbrugg, Switzerland).

TABLE 2
primer Mix VH-back
(SEQ ID NO: 50-68)
GTGATC GCC ATG GCG TCG ACC GAK GTR MAG CTT CAG GAG TC
GTGATC GCC ATG GCG TCG ACC GAG GTB CAG CTB CAG CAG TC
GTGATC GCC ATG GCG TCG ACC CAG GTG CAG CTG AAG SAR TC
GTGATC GCC ATG GCG TCG ACC GAG GTC CAR CTG CAA CAR TC
GTGATC GCC ATG GCG TCG ACC CAG GTY CAG CTB CAG CAR TC
GTGATC GCC ATG GCG TCG ACC CAG GTY CAR CTG CAG CAR TC
GTGATC GCC ATG GCG TCG ACC CAG GTC CAC GTG AAG CAR TC
GTGATC GCC ATG GCG TCG ACC GAG GTG AAS STG GTG GAR TC
GTGATC GCC ATG GCG TCG ACC GAV GTG AWG STG GTG GAG TC
GTGATC GCC ATG GCG TCG ACC GAG GTG CAG STG GTG GAR TC
GTGATC GCC ATG GCG TCG ACC GAK GTG CAM CTG GTG GAR TC
GTGATC GCC ATG GCG TCG ACC GAG GTG AAG CTG ATG GAR TC
GTGATC GCC ATG GCG TCG ACC GAG GTG CAR CTT GTT GAR TC
GTGATC GCC ATG GCG TCG ACC GAR GTR AAG CTT CTC GAR TC
GTGATC GCC ATG GCG TCG ACC GAA GTG AAR STT GAG GAR TC
GTGATC GCC ATG GCG TCG ACC CAG GTT ACT CTR AAA SAR TC
GTGATC GCC ATG GCG TCG ACC CAG GTC CAA CTV CAG CAR CC
GTGATC GCC ATG GCG TCG ACC GAT GTG AAC TTG GAA SAR TC
GTGATC GCC ATG GCG TCG ACC GAG GTG AAG GTC ATC GAR TC

TABLE 3
primer Mix VH-FOR
(SEQ ID NO: 69-72)
CCTCCACCACTCGAGCC CGA GGA AAC GGT GAC CGT GGT
CCTCCACCACTCGAGCC CGA GGA GAC TGT GAG AGT GGT
CCTCCACCACTCGAGCC CGC AGA GAC AGT GAC CAG AGT
CCTCCACCACTCGAGCC CGA GGA GAC GGT GAC TGA GGT

TABLE 4
primer Mix VL-BACK
(SEQ ID NO: 73-92)
GGCGGTGGC GCT AGC GAY ATC CAG CTG ACT CAG CC
GGCGGTGGC GCT AGC CAA ATT GTT CTC ACC CAG TC
GGCGGTGGC GCT AGC GAY ATT GTG MTM ACT CAG TC
GGCGGTGGC GCT AGC GAY ATT GTG YTR ACA CAG TC
GGCGGTGGC GCT AGC GAY ATT GTR ATG ACM CAG TC
GGCGGTGGC GCT AGC GAY ATT MAG ATR AMC CAG TC
GGCGGTGGC GCT AGC GAY ATT CAG ATG AYD CAG TC
GGCGGTGGCGCT AGC GAY ATY CAG ATG ACA CAG AC
GGCGGTGGC GCT AGC GAY ATT GTT CTC AWC CAG TC
GGCGGTGGCGCT AGC GAY ATT GWG CTS ACC CAA TC
GGCGGTGGC GCT AGC GAY ATT STR ATG ACC CAR TC
GGCGGTGGC GCT AGC GAY RTT KTG ATG ACC CAR AC
GGCGGTGGCGCT AGC GAY ATT GTG ATG ACB CAG KC
GGCGGTGGC GCT AGC GAY ATT GTG ATA ACY CAG GA
GGCGGTGGC GCT AGC GAY ATT GTG ATG ACC CAG WT
GGCGGTGGC GCT AGC GAY ATT GTG ATG ACA CAA CC
GGCGGTGGCGCT AGC GAY ATT TTG CTG ACT CAG TC
GGCGGTGGC GCT AGC GAA ACA ACT GTG ACC CAG TC
GGCGGTGGCGCT AGC GAA AAT GTK CTS ACC CAG TC
GGCGGTGGCGCT AGC CAG GCT GTT GTG ACT CAG GAA TC

TABLE 5
primer Mix VL-FOR
(SEQ ID NO: 93-96)
ATGCTGAC GC GGC CGC ACG TTT KAT TTC CAG CTT GG
ATGCTGAC GC GGC CGC ACG TTT TAT TTC CAA CTT TG
ATGCTGAC GC GGC CGC ACG TTT CAG CTC CAG CTT GG
ATGCTGAC GC GGC CGC ACC TAG GAC AGT CAG TTT GG

TABLE 6
sequencing primers
(SEQ ID NO: 97-100)
M13-Fwd
GTAAAACGACGGCCAGT
M13-Rev
AACAGCTATGACCATG
T7
TAATACGACTCACTATAGG
SP6
GATTTAGGTGACACTATAG

### EXAMPLE 3

### Biological Characterization of Anti-Human OX40 Antibodies

### OX40-Specific Antibody Detection ELISA

Antibody titers, specificity and production by hybridomas and recombinant antibody candidates were determined by a direct ELISA. In brief, 96 well-microtiter plates (Costar USA, distributor VWR AG, Nyon, Switzerland) were coated with 100 μl of recombinant human OX40-his at 2 μg/ml in PBS (see example 1 for the generation of the OX40-his protein). Plates were incubated overnight at 4° C. and were then blocked with PBS 2% BSA (Bovine Serum Albumine, PAA Laboratories, Pasching, Austria) at room temperature (RT) for one hour. The blocking solution was removed and the hybridoma supernatants or purified antibodies were added. The plates were incubated at RT for 30 minutes, then washed nine times with PBS 0.01% Tween-20 (Sigma-Aldrich Chemie GmbH, Buchs, Switzerland) and a Horseradish Peroxidase (HRP) labeled-Goat anti-mouse H+L-detection antibody (Sigma-Aldrich Chemie GmbH, Buchs, Switzerland) was added at a dilution of 1:1000. To detect recombinant chimeric antibodies (see example 2) that possess a human Fc, a HRP-labeled rabbit anti human IgG antibody (Sigma-Aldrich Chemie GmbH, Buchs, Switzerland) at a dilution of 1:1000 was used as the detection antibody. Plates were incubated for 30 minutes at RT, washed nine times with PBS 0.01% Tween-20 and the TMB substrate (Bio-rad Laboratories AG, Reinach, Switzerland) was added to the plates and the reaction stopped after six minutes by adding H₂SO₄. Absorbance was then read at 450 nm by a microplate reader (Biotek, USA; distributor: WITTEC AG, Littau, Switzerland). Amongst positive clones, hybridoma 7H11 and 2H6 were selected, coding DNA sequences of their variable domains were obtained and mouse-human IgG1 chimeras were prepared as described in example 2.

### EXAMPLE 4: HUMANIZATION AND OPTIMIZATION OF MOUSE 7H11 ANTIBODY

Humanizing the anti-human OX40 mouse antibody 7H11 including selection of human acceptor frameworks, back mutations, and mutations that substantially retain and/or improve the binding and properties of human CDR-grafted acceptor frameworks while removing potential post-translational modifications is described herein. The mouse 7H11 antibody has variable heavy chain domain sequence set forth in SEQ ID NO: 2 and variable light chain domain sequence set forth in SEQ ID NO: 3.

### Methods

### Recombinant Production of Antibodies

Coding DNA sequences (cDNAs) for the different VH and VL domains were synthesized in a scFv format by GENEART AG (Regensburg, Germany) thereby allowing for a single DNA sequence to encompass both variable domains. Individual variable domain cDNAs were retrieved from this scFv construct by PCR, and further assembled upstream of their respective constant domain cDNA sequence(s) using PCR assembly techniques. Finally, the complete heavy and light chain cDNAs were ligated in independent vectors that are based on a modified pcDNA3.1 vector (Invitrogen, CA, USA) carrying the CMV promoter and the Bovine Growth Hormone poly-adenylation signal. The light chain specific vector allowed expression of kappa isotype light chains by ligation of the light chain variable domain cDNA of interest in front of the kappa light chain constant domain cDNA using BamHI and BsiWI restriction enzyme sites; while the heavy chain specific vector was engineered to allow ligation of the heavy chain variable domain cDNA of interest in front of the cDNA sequence encoding the IGHG1 CH1, IGHG1 hinge region, IGHG1 CH2, and IGHG1 CH3 constant domains using BamHI and SalI restriction enzyme sites. In both heavy and light chain expression vectors, secretion was driven by the mouse VJ2C leader peptide containing the BamHI site. The BsiWI restriction enzyme site is located in the kappa constant domain; whereas the SalI restriction enzyme site is found in the IGHG1 CH1 domain.

Antibodies were transiently produced by co-transfecting equal quantities of heavy and light chains vectors into suspension-adapted HEK293-EBNA1 cells (ATCC® catalogue number: CRL-10852) using polyethylenimine (PEI, Sigma, Buchs, Switzerland). Typically, 100 ml of cells in suspension at a density of 0.8-1.2 million cells per ml is transfected with a DNA-PEI mixture containing 50 μg of expression vector encoding the heavy chain and 50 μg of expression vector encoding the light chain. When recombinant expression vectors encoding antibody genes are introduced into the host cells, antibodies are produced by further culturing the cells for a period of 4 to 5 days to allow for secretion into the culture medium (EX-CELL 293, HEK293-serum-free medium; Sigma, Buchs, Switzerland), supplemented with 0.1% pluronic acid, 4 mM glutamine, and 0.25 μg/ml geneticin).

The humanized antibodies were purified from cell-free supernatant using recombinant protein-A streamline media (GE Healthcare Europe GmbH, Glattbrugg, Switzerland), and buffered exchanged into phosphate buffer saline prior to assays.

### Affinity Measurements on HPB-ALL Cells by FACS

HPB-ALL cells (DSMZ, Braunschweig, Germany, Cat. No: ACC483) were used as a human OX40 positive cell line for FACS staining. HPB-ALL were maintained in RPMI 1640 supplemented with 10% FCS and 100 U/ml Penicillin and 100 μg/ml streptomycin. 4×10e5 HPB-ALL cells in FACS buffer (PBS supplemented with 1% BSA and 0.1% sodium azide) were incubated for 45 min on ice with the anti-OX40 antibody of interest kept at a concentration of 10 μg/ml An irrelevant human IgG1 was used as an isotype control; the cells were incubated with a 1/200 dilution of anti-Human Fc-PE (EBioscience, Vienna, Austria) for 45 min on ice. Cells were then washed again and resuspended in 200 μl FACS buffer. The relative mean fluorescence of each sample was measured on a FACSCalibur instrument (BD Biosciences, Allschwil, Switzerland).

### Affinity Measurements by SPR

SPR analysis was used to measure the association and dissociation rate constants for the binding kinetics of the anti-OX40 antibodies. The binding kinetics were measured on a BIAcore 2000 (BIAcore-GE Healthcare Europe GmbH, Glattbrugg, Switzerland) at room temperature and analyzed with the BiaEvaluation software (v4.1, GE Healthcare Europe GmbH).

Measurements were performed on CM5 sensor chips (Biacore 2000, GE Healthcare Europe GmbH, Cat. No: BR-1000-14) individually coupled with Protein A (Sigma, Buchs, Switzerland, Cat. No: P7837) using a commercial amine coupling kit (GE Healthcare Europe GmbH, Cat. No: BR-1000-50). 200-600 RUs of humanized antibody were captured. Dilution series of OX40-his were injected at a flow rate of 10 μl/min in HBS-EP buffer (GE Healthcare Europe GmbH, Cat. No: BR1001-88). After each binding event, the surface was regenerated with 10 μl of glycine buffer pH 1.5. Experimental data were processed using a 1:1 Langmuir model with local Rmax. The dissociation time was about 7 min. Measurements were performed in duplicates or triplicates and included zero-concentration samples for referencing. Both Chit and residual values were used to evaluate the quality of a fit between the experimental data and individual binding models.

### Thermostability Assessment by Differential Scanning Calorimetry

The thermal stabilities of the humanized antibodies were measured using differential scanning calorimetry (DSC). Monoclonal antibodies melting profiles are characteristic of their isotypes (Garber and Demarest (2007), BBRC 355:751-7), however the mid-point melting temperature of the FAB fragment can be easily identified even in the context of a full-length IgG. Such mid-point melting of FAB portion was used to monitor monoclonal stability of the humanized candidates.

Calorimetric measurements were carried out on a VP-DSC differential scanning microcalorimeter (Malvern Instruments Ltd, Malvern, UK). The cell volume was 0.128 ml, the heating rate was 200° C./h, and the excess pressure was kept at 65 p.s.i. All antibodies were used at a concentration of 1 mg/ml in PBS (pH 7.4). The molar heat capacity of antibody was estimated by comparison with duplicate samples containing identical buffer from which the antibody had been omitted. The partial molar heat capacities and melting curves were analyzed using standard procedures. Thermograms were baseline corrected and concentration normalized before being further analyzed using a Non-Two State model in the software Origin v7.0.

### Results

### Design of the Reshaped Variable Regions

Homology matching was used to select the human acceptor frameworks of the CDRs of the mouse 7H11 antibody. Databases (e.g. a database of germline variable genes from the immunoglobulin loci of human and mouse, the IMGT database (the international ImMunoGeneTics information System®; Lefranc M P et al., Nucleic Acids Res, 27(1):209-12 (1999); Ruiz M et al., Nucleic Acids Res, 28(1):219-21 (2000); Lefranc M P, Nucleic Acids Res, 29(1):207-9 (2001); Lefranc M P, Nucleic Acids Res, 31(1):307-10 (2003); Lefranc M P et al., Dev Comp Immunol, 29(3):185-203 (2005); Kaas Q et al., Briefings in Functional Genomics & Proteomics, 6(4):253-64 (2007)) or the VBASE2 (Retter I. et al, 2005, Nucleic Acids Res., 33, Database issue D671-D674), or the Kabat database (Johnson G. et al, 2000, Nucleic Acids Res., 28, p 214-218)) or publications (e.g., Kabat et al, Sequences of Proteins of Immunological Interest, 1992)) may be used to identify the human subfamilies to which the murine heavy and light chain V regions belong and determine the best-fit human germlime framework to use as the acceptor molecule for the mouse CDRs. Selection of heavy and light chain variable sequences (VH and VL) within these subfamilies to be used as acceptor may be based upon sequence homology and/or a match of structure of the CDR1 and CDR2 regions to help preserve the appropriate relative presentation of the six CDRs after grafting.

For example, use of the IMGT database indicates good homology between the 7H11 heavy chain variable domain framework and the members of the human heavy chain variable domain subfamily 1. Highest homologies and identities of both CDRs and framework sequences were observed for germline sequences: IGHV1-3*01 (SEQ ID NO: 4), IGHV1-2*02 (SEQ ID NO: 5), and IGHV1-46*01(SEQ ID NO: 6), all of which having sequence identity above 68% for the whole sequence up to CDR3. IGHV1-8*01 (SEQ ID NO: 7) had a lower sequence identity (66.3%).

Using the same approach, 7H11 light chain variable domain sequence showed good homology to the members of the human light chain variable domain kappa subfamily 3 and 4. Highest homologies and identities of both CDRs and framework sequences were observed for germline sequences: IGKV4-1*01 (SEQ ID NO: 8) (81.2% homology), IGKV3D-7*01 (SEQ ID NO: 9) (67.3% homology), IGKV3D-15*01 (SEQ ID NO: 10) (67.3% homology), and IGKV3-20*01 (SEQ ID NO: 11) (65.3% homology).

Best matching JH and JK segment sequences to the human acceptor framework were identified from the IMGT searches mentioned above.

As starting point to the humanization process, the four variable heavy and light chain domains stated above were selected as acceptors to the mouse 7H11 CDRs. A first set of 16 humanized antibodies of human gamma one isotype were prepared. These first humanized candidates were assessed for transient expression in HEK293E cells and binding to HB-ALL cell by flow cytometry (Table 7).

TABLE 7
Characterization of the first humanized 7H11 antibody candidates
(IgG1). FACS staining of anti-OX40 antibodies on HPB-ALL cell
line. MFI values correspond to antibodies mid-point fluorescence
measured by flow-cytometry using 10 μg/ml of antibody candidate.
Transient expression yields are reported in mg per L of culture.
Original human germline frameworks are indicated.

| | | VL | | | |
|---|---|---|---|---|---|
| | | VL1 | VL2 | VL3 | VL4 |
| | | IGKV4- | IGKV3D- | IGKV3D- | IGKV3- |
| VH | | 1*01 | 7*01 | 15*01 | 20*01 |
| VH1 | Yield | 41 | 45 | 3 | 2 |
| IGHV1-3*01 | MFI | 119.7 | 60 | 79.2 | 69.7 |
| VH2 | Yield | 51 | 7 | 2 | 26 |
| IGHV1-2*02 | MFI | 120 | 67.8 | 82.5 | 185.5 |
| VH3 | Yield | 31 | 17 | 2 | 35 |
| IGHV1-46*01 | MFI | 121.3 | 19.7 | 44.9 | 34.3 |
| VH4 | Yield | 34 | 8 | 1.5 | 20 |
| IGHV1-8*01 | MFI | 34.1 | 17.8 | 30.3 | 20.2 |

| | | |
|---|---|---|
| Isotype | MFI | 16.9 |
| control | | |
| Chimeric | MFI | 132.2 |
| 7H11 | | |
| IgG1 | | |

Best humanized candidates were antibodies VH1/VL1, VH2/VL1, and VH3/VL1. These antibodies exhibited FACS staining levels close to the level observed for the parental mouse antibody with expression yields above the remainder of the candidates.

The three candidates were further assayed by SPR for affinity ranking (Table 8). Surprisingly, the humanized VH2/VL1 IgG1 antibody was found to have superior affinity (i.e. lower KD) compared to the chimeric 7H11 antibody. In addition, expression yield, apparent affinity for HBP-ALL cells, and Fab stability were comparable to the other two variants.

TABLE 8
Characterization of the best first-graft humanized antibodies.
Affinity constants measured by SPR and Fab mid-point denaturation
temperatures measured by DSC are shown.

| | Chimeric | Humanized | Humanized | Humanized |
|---|---|---|---|---|
| | 7H11 | VH1-VL1 | VH2-VL1 | VH3-VL1 |
| | IgG1 | IgG1 | IgG1 | IgG1 |
| KD | 50 | 35 | 21 | 80 |
| (nM) | | | | |
| ka | 1.16xe4 | 1.67xe4 | 5.85xe4 | 6.25xe4 |
| (1/Ms) | | | | |
| kd | 6.6xe−4 | 5.8xe−4 | 1.3xe−3 | 5.5xe−3 |
| (1/s) | | | | |
| Fab Tm | 68 | 76.9 | 77 | 77.4 |
| (° C.) | | | | |

Based on its good binding, expression and Fab stability, the VH2/VL1 antibody was selected for further affinity improvement via the process known as back mutagenesis wherein amino acids from the mouse antibody sequence are introduced in the humanized antibody sequence. It was thought that affinity could be further improved by the process regardless of the fact that the VH2/VL1 antibody had better affinity than its parental mouse antibody.

### Back Mutations of Grafted Human Frameworks

The process of back mutation necessitates the identification and the selection of critical framework residues from the mouse antibody that need to be retained in order to preserve or improve affinity while at the same time minimizing potential immunogenicity in the humanized antibody. To identify residues that may impact the most CDR conformation and/or inter-variable domain packing, a 3D model for the VH2/VL1 pair of variable domains was calculated using the structure homology-modelling server SWISS-MODEL (Arnold K et al., (2006) Bioinformatics 22(2):195-201; http://swissmodel.expasy.org) set in automated mode. Model analysis allowed the selection of a subset of positions based on their putative influence on CDR regions and/or heavy chain-light chain variable domain packing. This subset of positions was selected out of the 26 possible back mutations found in the variable heavy chain, and consisted of positions: 37, 58, 60, 61, 85, 89, and 91 (Kabat numbering) (Table 9).

TABLE 9
Details of the positions selected for back mutation between
the humanized VH2/VL1 candidate and the mouse 7H11 antibody.

| | | | Spatial location |
|---|---|---|---|
| VH Kabat number | Antibody | Amino acid | in VH/VL interface |
| 37 | 7H11 | M | Middle interface |
| | VH2 | V | |
| 58 | 7H11 | K | Middle interface |
| | VH2 | N | |
| 60 | 7H11 | N | Bottom interface |
| | VH2 | A | |
| 61 | 7H11 | E | Bottom interface |
| | VH2 | Q | |
| 85 | 7H11 | E | Bottom interface |
| | VH2 | D | |
| 89 | 7H11 | 1 | Middle/bottom interface |
| | VH2 | V | |
| 91 | 7H11 | F | Middle interface |
| | VH2 | Y | |

Further humanized candidates based on these single back mutations were prepared in the context of the VH2/VL1 antibody sequences using standard PCR mutagenesis and the methods described above. Humanized antibody candidates were then assayed for their binding affinity by SPR and Fab thermal stability by DSC. Production yields, binding affinities, and Fab mid-points of thermal unfolding are shown in Table 10. Out of the seven antibodies tested, the N58K back mutation significantly improved affinity while maintaining good Fab thermal stability and expression. Consequently, the humanized VH2-N58K/VL1 antibody was selected for further optimization.

TABLE 10
Characterization of the humanized
VH2/VL1 back mutated antibodies.

| Antibody | Expression (mg/L) | KD (nM) | Fab Tm (° C.) |
|---|---|---|---|
| 7H11 (chimeric) | Not measured | 50 | 68.0 |
| VH2 V37M/VL1 | 12 | 2.87 | 70.3 |
| VH2 N58K/VL1 | 24 | 0.77 | 72.0 |
| VH2 A60N/VL1 | 14 | ND | 72.5 |
| VH2 Q61E/VL1 | 23 | ND | 71.3 |
| VH2 D85E/VL1 | 26 | ND | 71.5 |
| VH2 V89I/VL1 | 21 | ND | 70.8 |
| VH2 Y91F/VL1 | 9 | ND | 72.0 |

### Removal of a Potential Isomerisation Site.

Sequence analysis of 7H11 VH2 N58K highlighted the presence of a putative aspartate isomerization site (DG) in 7H11 CDRH2 at position 54 and 55 (Kabat numbering). To abrogate this isomerization site, site-directed mutagenesis was performed to replace 7H11 aspartate 54 residue by negatively charged or neutral polar amino acids like glutamate, serine and threonine and 7H11 glycine 55 by alanine. Using PCR assembly techniques, mutations D54E, D54S, D54T and G55A were introduced in the cDNA of 7H11 VH before ligation in a vector based on a modified pcDNA3.1 vector (Invitrogen, CA, USA) carrying the CMV promoter and a Bovine Growth Hormone poly-adenylation signal.

Using these approach, several vectors were generating encoding humanized 7H11 VH D54E (Humanized 7H11-VH2 N58K-D54E), 7H11 VH D54S (Humanized 7H11-VH2 N58K-D54S), 7H11 VH D54T (Humanized 7H11-VH2 N58K-D54T) and 7H11 VH G55A (Humanized 7H11-VH2 N58K-G55A). The parental sequence and variants of humanized 7H11 VH were co-transfected with 7H11 light chain in HEK293-EBNA1 cells. Cell supernatant were then collected 4 days after transfection for further purification using protein A. Tested mutations did not change 7H11 expression in mammalian cell as compared to the parental antibody (table 10).

To determine if these mutations could have changed antibody thermal stability, differential scanning fluorimetry was performed. Antibodies in PBS were first mixed with a 10× concentrated solution of SYPRO orange (Thermo Fisher Scientific, Ecublens, Switzerland) at a concentration of 250 μg/ml in a final volume of 20 μl. To record protein unfolding, samples were then exposed to an incremental increase of the temperature in a Rotor-Gene Q2plex HRM (QIAGEN, Hilden, Germany), thermal unfolding was followed by the presence of the SYPRO orange dye, whose fluorescence is quenched in polar environments but strongly emits a fluorescent signal when exposed to hydrophobic surroundings like the hydrophobic core of proteins upon unfolding. Recorded fluorescence signals were similar for both the parental and mutated forms of 7H11 indicating that mutations introduced in humanized 7H11 VH did not change antibody thermal stability (table 11).

Finally, Surface Plasmon Resonance analyses were applied to control antibody variant affinities as described earlier. Results in table 11 show that mutations introduced in 7H11 did not change antibody affinity.

TABLE 11
Summary of the humanized 7H11 variants
after isomerization site removal

| Antibody | Expression (mg/L) | KD (nM) | Fab Tm (° C.) |
|---|---|---|---|
| Humanized 7H11- | 39 | 16 | 74.2 |
| VH2 N58K | | | |
| Humanized 7H11- | 44 | 9 | 73.5 |
| VH2 N58K-D54E | | | |
| Humanized 7H11- | 37 | 49 | 73.5 |
| VH2 N58K-D54S | | | |
| Humanized 7H11- | 44 | 32 | 73.5 |
| VH2 N58K-D54T | | | |
| Humanized 7H11- | 44 | ND | 74 |
| VH2 N58K-G55A | | | |

### EXAMPLE 5: HUMANIZATION AND OPTIMIZATION OF MOUSE 2H6 ANTIBODY

### Humanization of Mouse Monoclonal 2H6

Humanization of the anti-human OX40 mouse antibody 2H6 including selection of human acceptor frameworks and mutations that substantially retain the binding properties of human CDR-grafted acceptor frameworks while removing potential post-translational modifications is described herein.

The human acceptor frameworks chosen to graft 2H6 CDRs were selected to confer maximum expression and/or stability to the humanized version of 2H6. Selection of human heavy and light chain variable sequences (VH and VL) to be used as acceptor may be based upon germlines with good biophysical properties (as documented in Ewert S et al., (2003) J. Mol. Biol, 325, 531-553) and/or pairing as found in natural antibody repertoire (as documented in Glanville J et al., (1999) Proc Natl Acad Sci USA, 106(48):20216-21; DeKosky B J et al., (2015) Nat Med, 21(1):86-91). Framework sequences known in the field for good paring and/or stability are the human IGHV3-23*01 (SEQ ID NO: 33) and IGKV1-16*01(SEQ ID NO: 34) which were used as acceptor frameworks for the 2H6 humanization.

A first humanized antibody of human gamma one isotype was prepared. The antibody encompassed a human-mouse hybrid heavy chain variable domain and a human-mouse hybrid light chain variable domain. The hybrid heavy chain variable domain was based on the human heavy chain variable domain IGHV3-23*01 wherein germline CDRH1 and H2 where respectively replaced for 2H6 CDRH1 and CDRH2. Best matching JH segment sequence to the human acceptor framework was identified from the IMGT database using homology search. The resulting human-mouse hybrid heavy chain variable sequence having human IGHV3-23*01 framework regions, 2H6 mouse CDRs, and best matching JH to human acceptor is referred herein as heavy chain variable domain VH1 with SEQ ID NO: 31.

Similarly, the human-mouse hybrid light chain variable domain used for this first humanized antibody candidate had human IGKV1-16*01 framework regions, 2H6 mouse CDRs, and best matching JK to human acceptor, and is refereed herein as light chain variable domain VL1 with SEQ ID NO: 32. The first humanized antibody encompassing VH1 and VL1 is abbreviated herein 2H6 VH1/VL1 antibody.

Production of the Humanized 2H6 scFv-Fc

Coding DNA sequences (cDNAs) for VH1 and VL1 were synthesized in a scFv format by GENEART AG (Regensburg, Germany) thereby allowing for a single DNA sequence to encompass both variable domains (SEQ ID NO: 35). The scFv cDNA was ligated in a vector based on a modified pcDNA3.1 vector (Invitrogen, CA, USA) described earlier. The scFv-Fc specific vector was engineered to allow ligation of the scFv cDNA of interest in front of the cDNA sequence encoding the human IGHG1 hinge region, IGHG1 CH2 and IGHG1 CH3 constant domains using BamHI and KpnI restriction enzyme sites. Secretion was driven by the mouse VJ2C leader peptide containing the BamHI site. An artificial Glycine-Threonine linker was introduced at the C-ter part of the scFv which contains the KpnI site.

The scFv-Fc was transiently produced by transfecting scFv-Fc vector into suspension-adapted HEK293-EBNA1 cells (ATCC® catalogue number: CRL-10852) as described earlier. Then, the scFv-Fc was purified from cell-free supernatant using recombinant protein-A streamline media (GE Healthcare Europe GmbH, Glattbrugg, Switzerland), and buffered exchanged into phosphate buffer saline prior to assays. Binding to human and cynomolgus monkey OX40 was measured by surface plasmon resonance as described below. The 2H6 humanized scFv encompassing VH1 and VL1 is abbreviated herein 2H6 scFv1.

Kinetic Binding Affinity Constants of the Humanized 2H6 scFv1-Fc for Human and Cynomolgus Monkey OX40 Receptor Extracellular Domain by Surface Plasmon Resonance (SPR)

Kinetic binding affinity constants (KD) were measured using recombinant histidine tagged human OX40 receptor extracellular domain and recombinant Fc fused cynomolgus monkey OX40 receptor extracellular domain captured on a CM5 chip and 2H6 scFv1-Fc and mouse chimeric 2H6 scFv-Fc as analytes. Measurements were conducted on a BIAcore T200 (GE Healthcare—BIAcore, Uppsala, Sweden) at room temperature, and analyzed with the Biacore T200 Evaluation software. A CM5 research grade sensor chip (GE Healthcare Europe GmbH, Glattbrugg, Switzerland; BR100530) was activated by injecting 35 μl of a 1:1 N-hydroxysulfosuccinimide (NHS)/1-Ethyl-3-[3-dimethylaminopropyl] carbodiimide Hydrochloride (EDC) solution (v/v; 5 μl/min flow-rate; on flow paths 1, 2, 3 and 4).

Cynomolgus monkey OX40R-Fc was diluted to a final concentration of 25 nM in acetate buffer pH 4.0 (GE, BR-1003-49) and subsequently immobilized on the previously activated CM5 sensor chip by injecting 10 μl on the flow path 2 (10 μl/min) which corresponds approximately to 600 response units (RUs). Human OX40R-His was diluted to a final concentration of 25 nM in acetate buffer pH 4.0 (GE, BR-1003-49) and subsequently immobilized on the previously activated CM5 sensor chip by injecting 45 μl on the flow path 4 (10 μl/min) which corresponds approximately to 400 response units (RUs). The OX40R-CM5 sensor chip was then deactivated by injecting 35 μl of ethanolamine solution (5μ1/min). Finally, two injections of 10 μl of glycine solution (GE, ref. BR-1003-54; 10 mM; pH 1.5) were performed to release non-crosslinked (human and cynomolgus monkey) OX40R molecules.

The 2H6 scFv-Fc was injected at different concentrations (0.78 nM to 0.2 μM) on the 4 flow-paths (flow-path 1 and 3 being used as references) at a 30 μl/min flow rate. After each binding event, surface was regenerated with glycine buffer pH 1.5 injected for 30 seconds (10 μl/min).

Measurements (sensorgram: fc2-fc1 and fc4-fc3) were best fitted with a 2:1 bivalent analyte model with mass transfer. Dissociation times were of at least 300-600 seconds. The Chi2 value represents the sum of squared differences between the experimental data and reference data at each point; while the plots of residuals indicate the difference between the experimental and reference data for each point in the fit. Both Chi2 and residual values were used to evaluate the quality of a fit between the experimental data and individual binding models.

Results shown in table 12 indicates that humanized 2H6 scFv1 has a similar affinity to human and cyno OX40 than the parental mouse 2H6 scFv

TABLE 12
Characterization of the humanized 2H6-scFv by SPR

| | mouse | humanized | mouse | humanized |
|---|---|---|---|---|
| | chimeric 2H6 | 2H6 | chimeric 2H6 | 2H6 |
| | scFv-Fc | scFv1-Fc | scFv-Fc | scFv1-Fc |
| Analyte | Human OX40 | | Cynomogus monkey OX40 | |
| KD (nM) | 62 | 27 | 39 | 18 |
| ka (1/Ms) | 6.8xe5 | 1.55xe5 | 5.79xe5 | 2.06xe5 |
| kd (1/s) | 2.2xe−2 | 7.41xe−4 | 3.19xe−4 | 5.86xe−4 |

2H6 Met Removed from JH

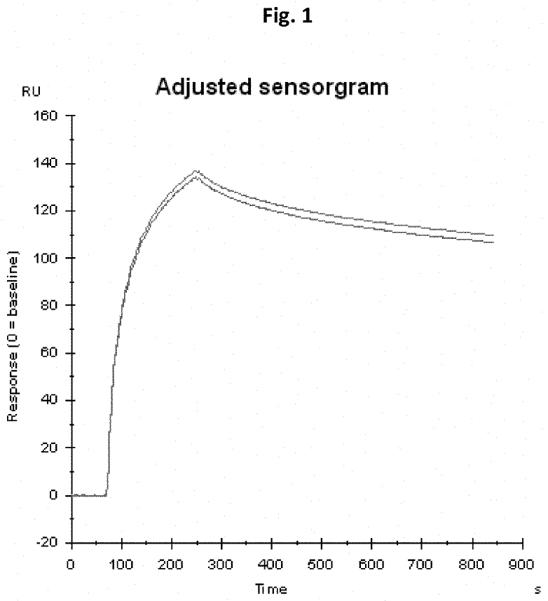

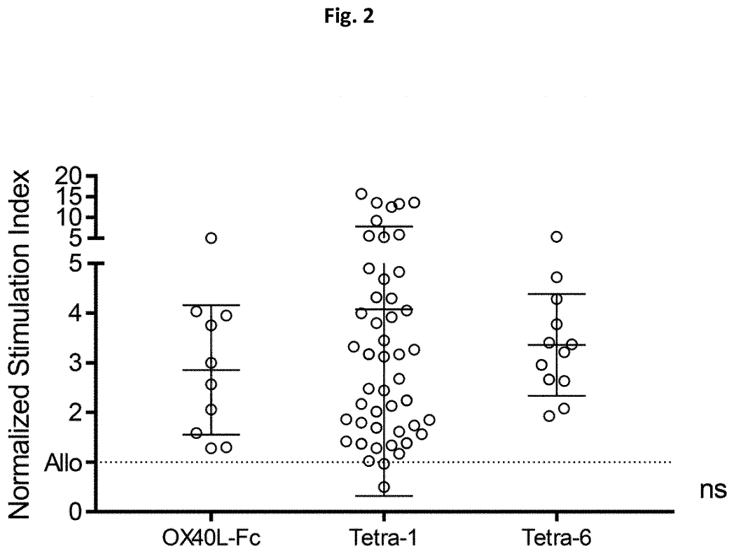

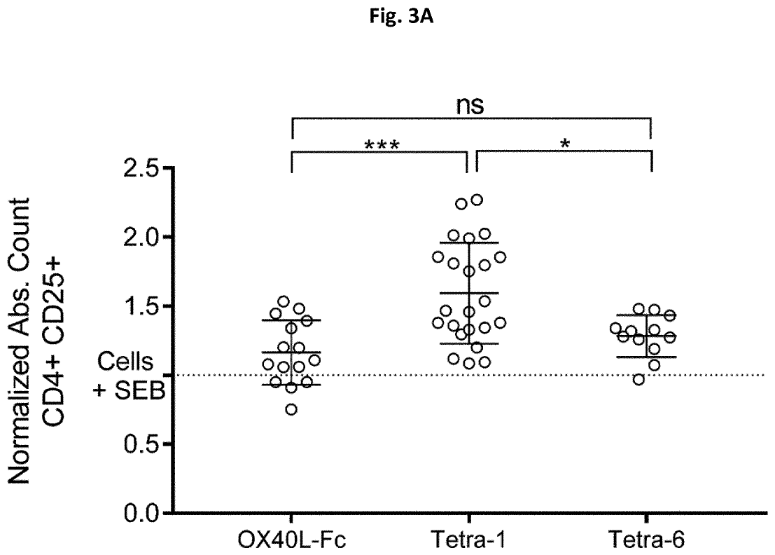

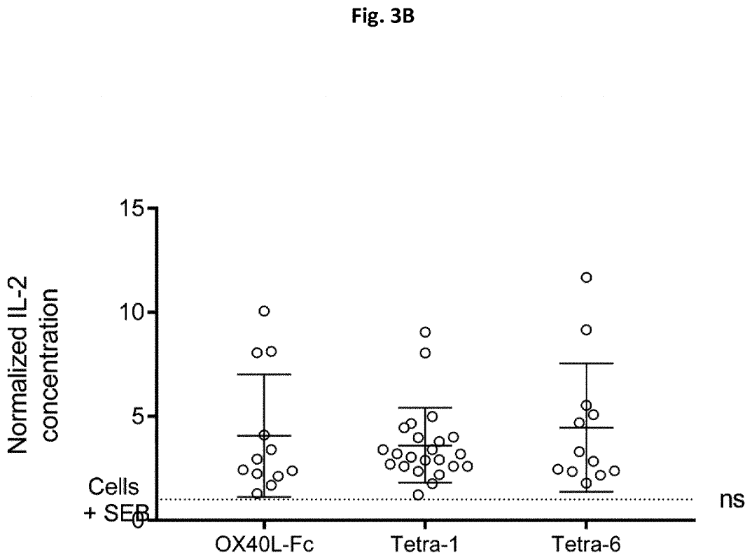

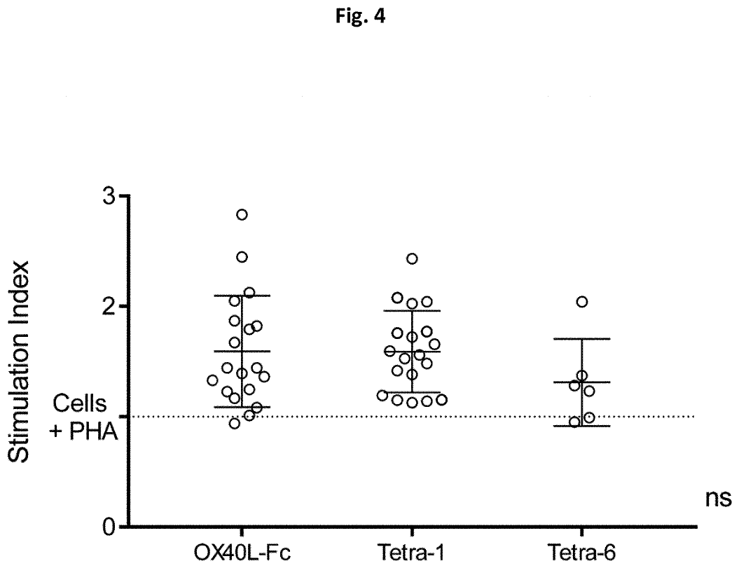

Sequence analysis of 2H6 scFv highlighted the presence of a putative oxydation site (Methionine 108) in the 2H6 VH JH region. To abrogate this potential oxydation site, site-directed mutagenesis was performed to replace 2H6 VH methionine 108 residue by a leucine amino acid. Using PCR assembly technique, VH mutation M108L (kabbat numbering) was introduced in the cDNA of 2H6 scFv before ligation in a vector based on a modified pcDNA3.1 vector as described earlier. Using this approach, a vector encoding humanized 2H6 scFv M108L (abbreviated 2H6 scFv2) was generated. The parental and mutated forms of 2H6 scFv-Fc were transfected in HEK293-EBNA1 cells as described earlier. Cell supernatant were then collected 4 days after transfection for further purification using protein A. Tested mutations did not change 2H6 scFv-Fc expression in mammalian cell as compared to the parental antibody (table 13). To determine if M108L mutation could have changed scFv thermal stability, differential scanning fluorimetry was performed as described earlier. Recorded fluorescence signals were similar for both the parental and mutated forms of 2H6 indicating that M108L mutation did not change scFv thermal stability (table 12). Finally, Surface Plasmon Resonance analyses were applied to control binding properties of 2H6 scFv2 variant as described earlier. Results in FIG. 1 show that mutations introduced in 2H6 scFv did not change its binding.

TABLE 13
Characterization of the impact of M108L
mutation on humanized 2H6-scFv

| | Expression | |
|---|---|---|
| Antibody | (mg/L) | scFv Tm (° C.) |
| 2H6 scFv1-Fc | 40 | 63 |
| 2H6 scFv2-Fc | 40 | 62.7 |

### EXAMPLE 7: ENGINEERING AND PRODUCTION OF TETRAVALENT ANTI-HUMAN OX40 ANTIBODIES

### First Tetravalent Molecules

The tetravalent format used is a whole IgG to which scFvs were connected via (Gly4Thr) linker at the C-terminus of the heavy chain. For the generation of a tetravalent antibody having 7H11 IgG1 fused to 2H6 scFv, coding DNA sequences (cDNAs) for humanized 7H11-VH2 N58K (SEQ ID NO: 21), VL1 (SEQ ID NO: 16) and 2H6 scFv1 were PCR amplified before digestion and ligation in vectors based on a modified pcDNA3.1 vector (Invitrogen, CA, USA) described earlier. The light chain specific vector was engineered to allow ligation of the VL cDNA of interest in front of the cDNA sequence encoding the human kappa constant domain using BamHI and BsiWI restriction enzyme sites. The heavy chain specific vector was engineered to allow ligation of the VH cDNA of interest in front of the cDNA encoding the IGHG1 hinge region, a modified IGHG1 CH2 domain with the L234A/L235A double mutation (LALA, Eu numbering, Hezareh M et al., (2001) J Virol, 75:12161-8) which reduces Fc-FcγRs interactions and a modified IGHG1 CH3 constant domain having a (Gly4Thr) linker in its C-terminal part using BamHI and SalI restriction enzyme sites. Then, scFv cDNA of interest was ligated after the IGHG1 CH3 constant domain and the (Gly4Thr) linker of the heavy chain specific vector using KpnI and NotI restriction enzyme sites. In both heavy and light chain expression vectors, secretion was driven by the mouse VJ2C leader peptide containing the BamHI site. The BsiWI restriction enzyme site is located in the kappa constant domain; whereas the SalI restriction enzyme site is found in the IGHG1 CH1 domain. The glycine-threonine linker contains the KpnI site while the NotI site is present before the Bovine Growth Hormone poly-adenylation signal found in the modified pcDNA3.1 vector encoding the heavy chain.

This tetravalent antibody (abbreviated Tetra-1) was transiently produced by co-transfecting equal quantities of the 7H11VL1 light chain and Tetra-1 heavy chain vectors into suspension-adapted HEK293-EBNA1 cells as described previously. The tetravalent antibody was purified from cell-free supernatant using recombinant protein-A streamline media (GE Healthcare Europe GmbH, Glattbrugg, Switzerland), and buffered exchanged into phosphate buffer saline prior to assays.

### Optimized 7H11×2H6 Tetravalent Antibodies:

In the tetravalent molecule described above, scFv is fused in C-terminus of the IgG. Therefore, the last C-terminal residue of the antibody is a Lysine naturally present in the JK region. To avoid C-ter lysine clipping of the tetravalent molecule in the circulation which could have an impact on the biology of the antibody, site directed was used to replace the C-ter Lysine of 2H6 scFv by a leucine residue. Using PCR assembly technique, VL mutation K107L (Kabbat numbering) was introduced in the cDNA of 2H6 scFv before ligation in the vector coding for the tetravalent antibody described above. The parental and mutated forms of tetravalent antibodies were transfected in HEK293-EBNA1 cells as described earlier. Cell supernatant were then collected 4 days after transfection for further purification using protein A. Tested mutation did not change tetravalent antibody expression in mammalian cell as compared to the parental antibody (table 14). To determine if K107L mutation could have changed C-terminal scFv thermal stability, differential scanning fluorimetry was performed as described earlier. Recorded fluorescence signals were similar for both the parental and mutated forms of 2H6 scFv indicating that K107L mutation did not change scFv thermal stability (table 13).

TABLE 14
Characterization of the impact of K107L
mutation on humanized Tetra-1 antibody

| | Expression | |
|---|---|---|
| Antibody | (mg/L) | C-ter 2H6 scFv Tm (° C.) |
| Tetra-1 | 33 | 60.5 |
| Tetra-1-K107L | 32 | 59.7 |

To finalize the optimization of the tetravalent antibody, 7H11 mutation D54E, 2H6 VH mutation M108L and VL mutation K107L were introduced in the vector coding for the tetravalent molecule heavy chain (abbreviated Tetra-6) by site-directed mutagenesis as previously described. Tetra-6 was produced by co-transfecting 7H11 VL1 light chain and Tetra-6 heavy chain vectors as shown before and purified from cell-free supernatant using recombinant protein-A streamline media.

### EXAMPLE 8: IN VITRO CHARACTERIZATION OF TETRAVALENT ANTI-HUMAN OX40 ANTIBODIES

### Example 8.1 Tetra-1 and Tetra-6 Display a Proliferative Effect in a Mixed Lymphocyte Reaction (MLR) Assay

PBMCs were isolated from citrated whole blood of healthy donors using ficoll density gradient. Monocytes were isolated from PBMCs using Monocyte isolation kit (Miltenyi) and cultured with GM-CSF at 50 ng/mL (R&D) and rhIL-4 at 20 ng/mL (R&D) for 7 days to differentiate them into dendritic cells (DC). The phenotype of dendritic cells was verified by flow cytometry using CD1c APC (eBioScience). On day 7, CD4 T cells (from an allogeneic donor) were isolated from PBMCs using the EasySep kit (StemCell Technologies). CD4 T cells (40,000 cells/well) and DC (8,000 cells/well) were co-cultured with antibodies at 80 nM for 6 days in complete media in a 96-well round-bottom plate in triplicate. On day 13, 3H-thymidine was added (Perkin Elmer, 0.5 μCi per well). Twenty hours after pulsing, cells were harvested and incorporated radioactivity was quantified on a Wallac beta counter. A normalized stimulation index (SI) was determined using this formula:

(Sample−Resp only)/(Allo−Resp only)

“Sample” corresponds to the counts of the conditions in which DC+CD4 T cells+tested antibody were co-cultured. “Resp only” corresponds to the counts of the condition in which only responder cells (CD4 T cells) were added. “Allo” corresponds to the condition in which DC (stimulator cells) and allogeneic CD4 T cells (responder cells) were co-incubated. Data were analyzed using Graphpad Prism 7 software; Statistical analysis was performed with a Mann-Whitney test (non-parametric test) or a Wilcoxon matched-pairs test. P<0.05 was considered as statistically significant.

The OX40L-Fc is a potent agonistic molecule that can efficiently engage and crosslink OX40 on surface of T cells (Müller FEBS J. 2008 May; 275(9):2296-304). In agreement with this feature, OX40L-Fc was able to increase a mixed-lymphocyte reaction. In this assay, both Tetra-1 and Tetra-6 enhanced the allogeneic response to a similar level as OX40L-Fc (differences between these three molecules not statistically significant; FIG. 1). A Wilcoxon matched-pairs test comparing SI of Tetra-1 and Tetra-6 tested in the same experiment showed that these two tetravalent molecules improved similarly the proliferation (data not shown). Therefore these results highlight that targeting OX40 with Tetra-1 and Tetra-6 provides a relevant immunostimulatory potential.

### Example 8.2 Tetra-1 and Tetra-6 Induce a Strong Immunostimulatory Effect in a Staphylococcal Enterotoxin B Stimulation Assay

Peripheral blood mononuclear cells (PMBC5) were harvested from blood filters obtained from La Chaux-de-Fonds Transfusion Center using ficoll density gradient isolation. PBMCs (105) were distributed in a 96-well round-bottom plate in triplicate. Staphylococcal enterotoxin B (SEB) at a final concentration of 50 or 100 ng/mL (suboptimal concentrations) and antibodies at 80 nM final concentration were added to the wells. Plates were incubated for 7 days at 37° C. in a CO2 incubator. IL-2 production in the culture supernatants was measured with Luminex using a ProcartaPlex kit (eBiosciences) on day 5. On day 7, cells were harvested and labeled with anti-human CD4 ECD and anti-human CD25 Pacific Blue (eBiosciences). Stained cells were resuspended in 100 μL of FACS buffer and analyzed by flow cytometry on CytoFLEX S (Beckman). Flow cytometry data were analyzed using CytExpert (Beckman). The gating strategy consisted in gating on living cells (based on FSC and SSC plots) cells, CD4 positive cells and subsequently on CD4+ CD25+. The total number of cells per well within this subset of interest was calculated using this formula:

(Volume used to resuspend cells*Number of events in the gate)/Sample volume acquired

Each data point was normalized to the condition in which PBMCs were incubated with SEB only (No antibody). Data were analyzed using Graphpad Prism 7 software and statistical analysis was performed with a Mann-Whitney test (non-parametric test). P<0.05 was considered as statistically significant.

In this assay, Tetra-1 was able to significantly increase the number of CD4+CD25+ cells (91% of the donors tested reached a 1.2 fold induction compared to SEB only; threshold arbitrarily defined) while OX40L-Fc and Tetra-6 did so to a lesser extent (67% and 88% respectively). The expression of CD25 defines activated T cells. The increase of CD25 expressing CD4 T cells can be due to an increase in the activation of T cells and/or an increase of proliferation of activated T cells. Similarly, the addition of Tetra-1, Tetra-6 and OX40L-Fc also substantially enhanced IL-2 production compared to SEB only. IL-2 production also indicates T cell activation and is linked directly to T cell proliferation. There was no statistical difference between the three molecules with this readout and at this stage of the experiment (day 5).

Overall, targeting OX40 in this superantigen-mediated PBMC stimulation strikingly improved T cell responses as visualized by enhanced cytokine production (IL-2) or enhanced T cell activation (CD25).

### Example 8.3 Tetra-1 Displays a Strong Immunostimulatory Effect in a PHA Stimulation Assay

PBMCs were prepared the same way as for the SEB assay. PBMCs (105) were distributed in a 96-well round-bottom plate in triplicate. PHA at 2 or 1 μg/mL final concentration and antibodies at 80 nM final concentration were added. Plates were incubated for 7 days at 37° C. in a CO2 incubator. Six days after the start of the assay, cells were pulsed with 0.5 μCi per well of 3H-thymidine (Perkin Elmer). Twenty hours after pulsing, cells were harvested and incorporated radioactivity was quantified on a Wallac beta counter. A stimulation index was determined using this formula:

Sample/PHA only

“Sample” corresponds to the counts of the condition PBMCs+PHA+tested antibody. “PHA only” corresponds to the counts of the condition in which PBMCs were cultured with PHA only (no antibody). Each data point was normalized to the condition in which PBMCs were incubated only with PHA (No antibody). Data were analyzed using Graphpad Prism 7 software. Statistical analysis was performed with a Mann-Whitney test (non-parametric test). P<0.05 was considered as statistically significant.

In this assay, Tetra-1 and Tetra-6 increased cell proliferation in response to a suboptimal concentration of PHA. No significant difference were detected between Tetra-1, Tetra-6 and OX40L-Fc.

### EXAMPLE 9: GENERATION OF DISULFIDE BOND STABILIZED TETRA-8 MOLECULE

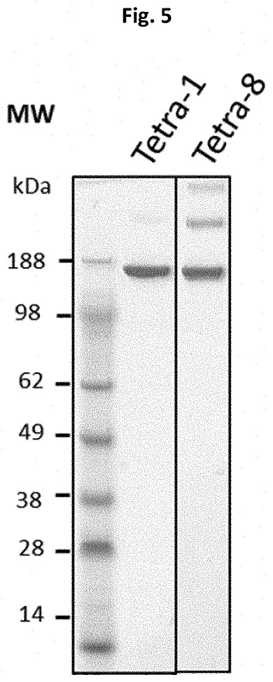

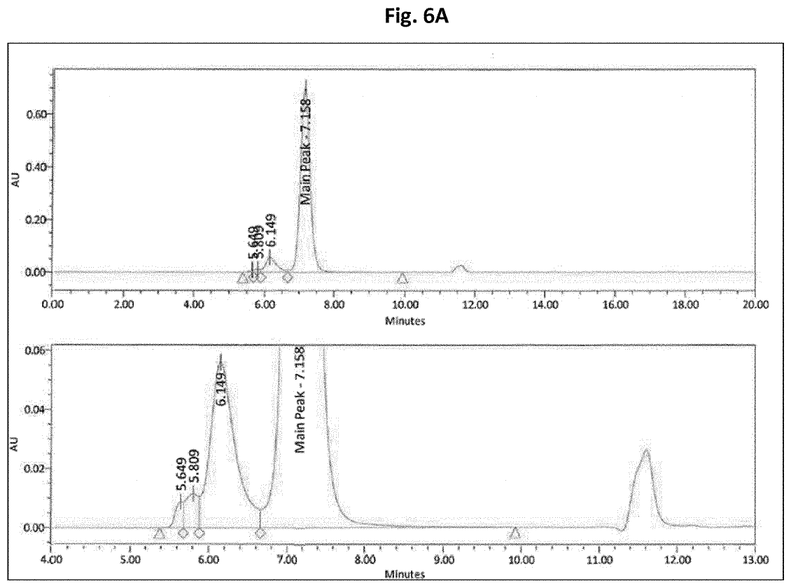

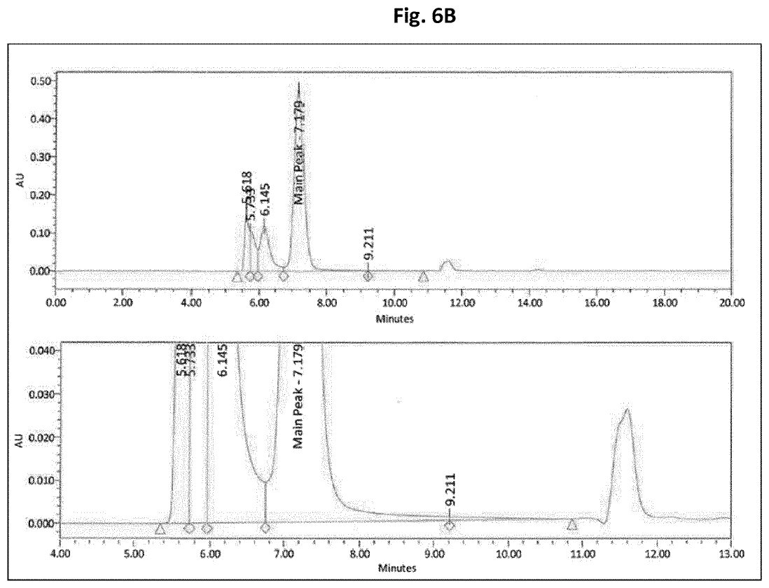

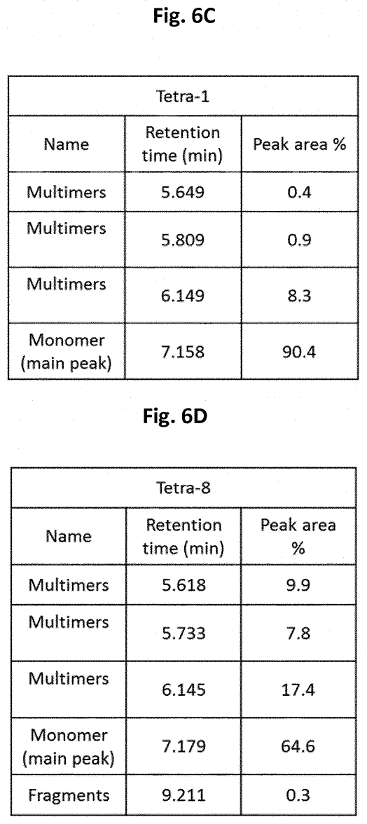

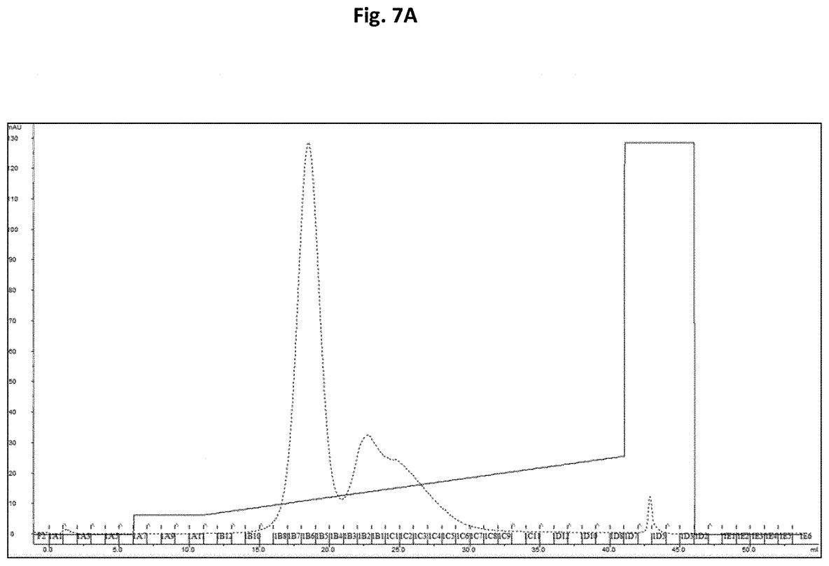

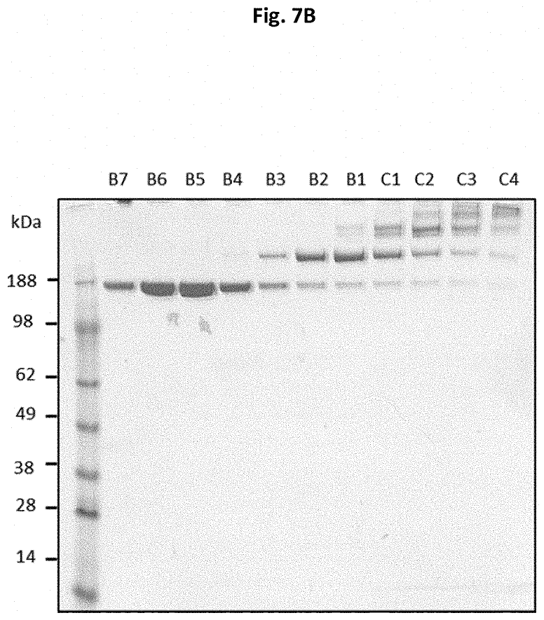

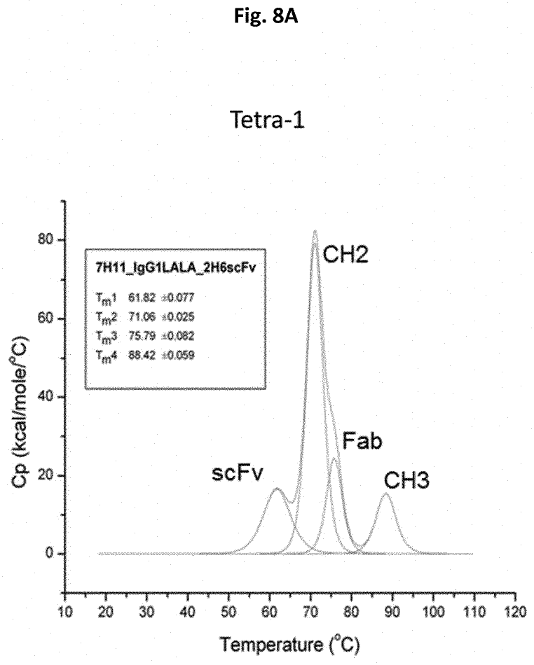

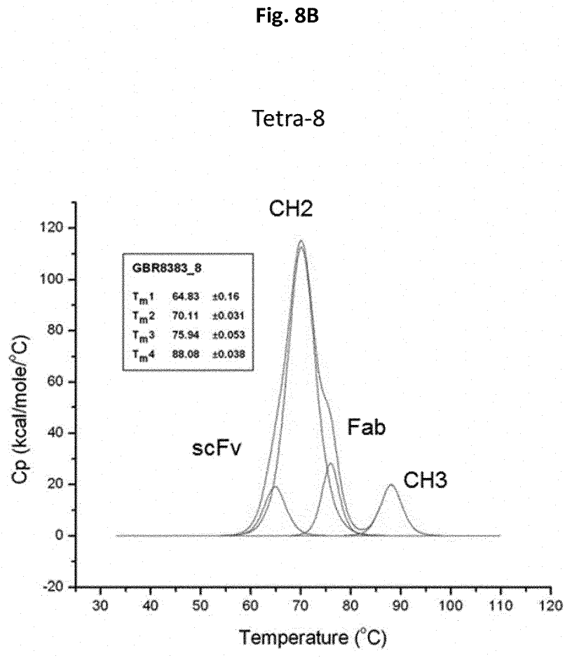

In order to further enhance the Tetra-6 molecule, the 2H6 scFv bearing VH mutation M108L and VL mutation K107L was engineered to increase its stability by introducing a disulphide bond between the VH and VL domains (Reiter Y et al., Nat Biotechnol., 14(10):1239-45, October 1996). Using PCR assembly technique, VH G44C and VL Q100C mutations were introduced in the cDNA of the mutated 2H6 scFv before ligation in the vector coding for a new tetravalent molecule heavy chain (abbreviated Tetra-8). Tetra-8 was then produced by co-transfecting 7H11 VL1 light chain (SEQ ID NO: 16) and Tetra-8 heavy chain (SEQ ID NO: 45) vectors as previously described. After protein A purification, molecule was analysed by non-reduced SDS-PAGE (FIG. 5) and SEC-HPLC (FIG. 6). Both analytical methods showed that disulfide bond engineering of the tetravalent molecule induces the formation of covalent multimers. To separate multimers from the monomer, an additional cation exchange purification step was carried it out. Briefly, a HiTrap SP HP column (GE Healthcare Europe GmbH, Glattbrugg, Switzerland) was first equilibrated using 50 mM Sodium Acetate pH5.5 buffer. Then, protein sample was loaded and molecules were separated using a gradient of 50 mM Sodium Acetate+1M NaCl pH 5.5 from 5% to 20% and 100% (FIG. 7). Fractions containing monomeric form of Tetra-8 were then pooled and submitted to buffer exchange against PBS (Gibco, ThermoFischer scientific, MA, USA). Finally, thermal stability of the monomeric Tetra-8 was assessed by Differential Scanning calorimetry (DSC) and compared to the Tetra-1 (FIG. 8). A clear increase of thermal stability (3° C.) of the mutated 2H6 scFv domain in Tetra-8 was measured indicating that disulfide-bond engineering was efficiently enhancing 2H6 scFv melting temperature.

### Biophysical Characterization of Tetra-8 Antibody

The affinity of the 2H6 scFv binder in Tetra-8 was determined by Biacore. To allow precise measurement, Tetra-8 molecule was digested with the FabALACTICA protease (Genovis AB, Lund, Sweden) to remove the 7H11 Fab. Briefly, Tetra-8 molecule was first submitted to buffer exchange in 150 nM Sodium Phosphate pH7.0 before addition of 1 FabALACTICA unit per μg of antibody. The antibody/protease mixture was incubated over night at 37° C. Then, this material was further purified using CaptureSelect™ FcXL Affinity Matrix resin (ThermoFischer scientific, MA, USA) to remove the 7H11 Fabs and the protease from the mixture while capturing the Fc-2H6 scFv fragments. The resin was washed with PBS and the specific Fc-2H6 fragment was then eluted with 0.1M Glycine pH3.0 and finally formulated in PBS pH7.4. Using anti-human IgG Fc immobilized on a CM5 chip, kinetic was measured by capturing 600 RU's of Fc-2H6 fragment and by injecting dilution series of OX40-CRD-Avi-his (SEQ ID NO: 159) at a flow rate of 30 μl/min in HBS-EP buffer (GE Healthcare Europe GmbH, Cat. No: BR1001-88). After each binding event, the surface was regenerated with 10 μl of MgCl2 (3M) buffer. Experimental data were processed using a 1:1 Langmuir model with local Rmax. The dissociation time was about 7 min. The measured affinity of the 2H6 scFv (VH/M108L-VL/K107L-disulfide engineered) fused in C-terminus of the Tetra-8 molecule is 60 nM which only represents a 2-fold loss compared to humanized 2H6 scFv1-Fc (table 6) and indicates that the 2H6 binding arms are functional.

### Anti-OX40 Antibodies

To assess Tetra-8 agonist activity, several known agonist anti-OX40 were produced. The 11D4 IgG2 (US 2012/0225086 A1), the 9B12 IgG1 (OX40mab24, US 2016/0137740 A1), the 106-222 IgG1 (US 2016/0068604 A1), the 1A7 IgG1 (WO 2015/153513 A1) and the pab1949 (US 2016/0347847 A1) heavy and light chain sequences were retrieved from their respective patent applications and were gene synthesized as cDNA by Geneart AG (ThermoFischer scientific, MA, USA). The heavy and light chain sequences were then ligated in independent vectors which are based on the modified pcDNA3.1 vector previously described. The vectors coding for the respective heavy and light chains of each antibody were co-transfected in HEK293-EBNA1 cells as described earlier (table 15). Cell supernatant were then collected 4 days after transfection for further purification using protein A. The domain antibody (dAb) sequences of Tetra-hzG3V9, Tetra-hz1D10v1, Hexa-hzG3V9 and Hexa-hz1D10v1 (WO 2017/123673 A2) were also gene synthesized by Geneart AG before cloning in frame of a mutated IgG1 Fc sequence (LALA) into the modified pcDNA3.1 vector. Vectors coding for respective molecules were then transfected alone in HEK293-EBNA1 cells and the supernatants were collected before protein A purification. Finally, the 7H11-VH2 N58K-D54E was cloned in frame of the human IgG1 or IgG1 LALA sequences and were combined to 7H11 VL1 for the production of 7H11 IgG1 or IgG1 LALA (table x). The 2H6 scFv1 was cloned in frame of the IgG1-Fc LALA domain for production of 2H6 scFv-Fc LALA. The 2H6 VH1 was cloned in frame of the human IgG1 or IgG1 LALA sequences. These sequences were co-transfected with 2H6 LC to produce 2H6 IgG1 and IgG1 LALA (Table 15).

TABLE 15
Combination of heavy and light chains for antibody production.
Heavy and light chains used for antibody production

| IgG/dAb-Fc | Heavy chain | Light chain |
|---|---|---|
| 11D4 IgG2 | 11D4 IgG2 HC | 11D4 LC |
| | (SEQ ID: 101) | (SEQ ID: 102) |
| 9B12 IgG1 | 9B12 IgG1 HC | 9B12 LC |
| | (SEQ ID: 103) | (SEQ ID: 104) |
| 106-222 IgG1 | 106-222 IgG1 HC | 106-222 LC |
| | (SEQ ID: 105) | (SEQ ID: 106) |
| 1A7 | 1A7 IgG1 HC | 1A7 LC |
| | (SEQ ID: 107) | (SEQ ID: 108) |
| pab1949 | pabl949 IgG1 HC | pabl949 LC |
| | (SEQ ID: 109) | (SEQ ID: 110) |
| Tetra-hz1D10v1 | Tetra-hz1D10v1 HC | — |
| | (SEQ ID: 111) | |
| Tetra-hzG3V9 | Tetra-hzG3V9 HC | — |
| | (SEQ ID: 112) | |
| Hexa-hz1D10v1 | Hexa-hz1D10v1 HC | — |
| | (SEQ ID: 113) | |
| Hexa-hzG3V9 | Hexa-hzG3V9 HC | — |
| | (SEQ ID: 114) | |
| 2H6 IgG1 | 2H6 IgG1 HC | 2H6 LC |
| | (SEQ ID: 115) | (SEQ ID: 116) |
| 2H6 IgG1 | 2H6 IgG1 LALA HC | 2H6 LC |
| LALA | (SEQ ID: 117) | (SEQ ID: 116) |
| 2H6 scFv-Fc | 2H6 scFv-Fc LALA HC | — |
| LALA | (SEQ ID: 118) | |
| 7H11 IgG1 | 7H11_v8 IgG1 HC | Humanized 7H11-VL1 |
| | (SEQ ID:119) | (SEQ ID: 16) |
| 7H11 IgG1 | 7H11 IgG1 LALA HC | Humanized 7H11-VL1 |
| LALA | (SEQ ID:120) | (SEQ ID: 16) |

### Chimeric OX40 Molecules for Antibody Epitope Mapping

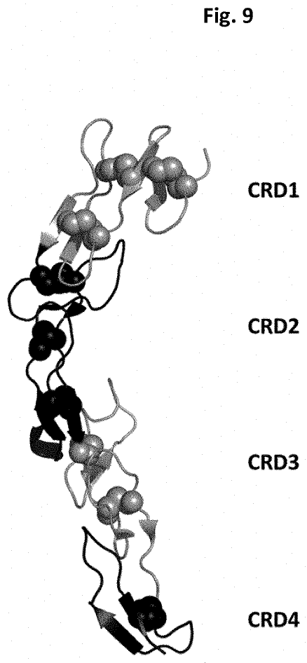

OX40 is a member of the TNFR superfamily which is characterized by the presence of four domains defined as cysteine-rich domain (CRD) in its extra-cellular part (FIG. 9). To determine the domains targeted by antagonist antibodies, OX40 chimeras must be designed and expressed to be used as tools for epitope mapping. The sequences of the extracellular domains of human, rat and cynomolgus monkey OX40 were retrieved from the Uniprot database (SEQ ID NOs: 1, 121, 122, respectively), gene synthesised by Geneart before cloning as Fc-fusion proteins (SEQ ID NOs: 123, 124, 125, respectively). These constructs were expressed in HEK293-EBNA1 cells and purified using protein A. Then, human and rat OX40-Fc were tested in ELISA and Biacore to determine antibody cross-reactivity. For the ELISA, 96 well-microtiter plates (Costar USA, distributor VWR AG, Nyon, Switzerland) were coated with 100 μl of recombinant human or rat OX40-Fc at 2 μg/ml in PBS. Plates were incubated overnight at 4° C. and were then blocked with PBS 2% BSA (Bovine Serum Albumine, PAA Laboratories, Pasching, Austria) at room temperature (RT) for one hour. The blocking solution was removed and the purified antibodies were added at 10 μg/ml in PBS 2% BSA. The plates were incubated at RT for 1 hour, then washed 5 times with PBS 0.01% Tween-20 (Sigma-Aldrich Chemie GmbH, Buchs, Switzerland). To detect recombinant antibodies that possess a human Fab, a peroxidase-conjugated Goat Anti-Human IgG, Fab Fragment Specific (Jackson ImmunoResearch, 109-035-006) diluted of 1:2000 in PBS 2% BSA was used as the detection antibody. Plates were incubated for 1 hour at RT, washed 5 times with PBS 0.01% Tween-20 and the TMB substrate (Bio-rad Laboratories AG, Reinach, Switzerland) was added to the plates and the reaction stopped after 5 minutes by adding H₂SO₄. Absorbance was then read at 450 nm by a microplate reader (Biotek, USA; distributor: WITTEC AG, Littau, Switzerland). Biacore experiments were specifically conducted with Hexa-hzG3V9 and Hexa-hz1D10v1 which are dAb-Fc fusion proteins. Briefly, 5 μl of Tetra-hz1D10v1 (5 μg/mL) was immobilized on the previously activated CM5 sensor chip by injecting to flow path 2 at a flow rate of 10 μl/min which corresponds approximately to 206 RU. Similarly, 15 μl of Tetra-hzG3V9 (5 μg/mL) was injected to flow path 4 at a flow rate of 10 μl/min which corresponds approximately to 251 RU. Binding of human and cyno OX40-Fc molecules was determined by successively injecting these proteins on the 4 flow-paths (flow-path 1 and 3 being used as references) at a concentration of 200 nM and a flow rate of 30 uL/min for 240 seconds. Regeneration between the two injections was done using 3M MgCl2, at 30 uL/min for 60 seconds. Using these two approaches, human/rat OX40 binding was assessed (table 16).

TABLE 16
Characterization of cross-reactivity of anti-OX40 antibodies
Cross-reactivity of anti-OX40 antibodies

| | Human OX40-Fc binding | Rat OX40-Fc binding |
|---|---|---|
| 2H6 | Yes | No |
| 7H11 | Yes | No |
| 9B12 | Yes | No |
| 11D4 | Yes | No |
| 106-222 | Yes | No |
| 1A7 | Yes | Yes |
| pab1949 | Yes | No |
| Tetra-hzG3V9 | Yes | No |
| Tetra-hz1D10v1 | Yes | No |

These experiments clearly demonstrated that most of these antibodies (at the exception of 1A7) were lacking cross-reactivity with rat OX40 indicating that this protein could be used to generate chimeras with human OX40 sequence for epitope mapping purposes.

### Epitope Mapping

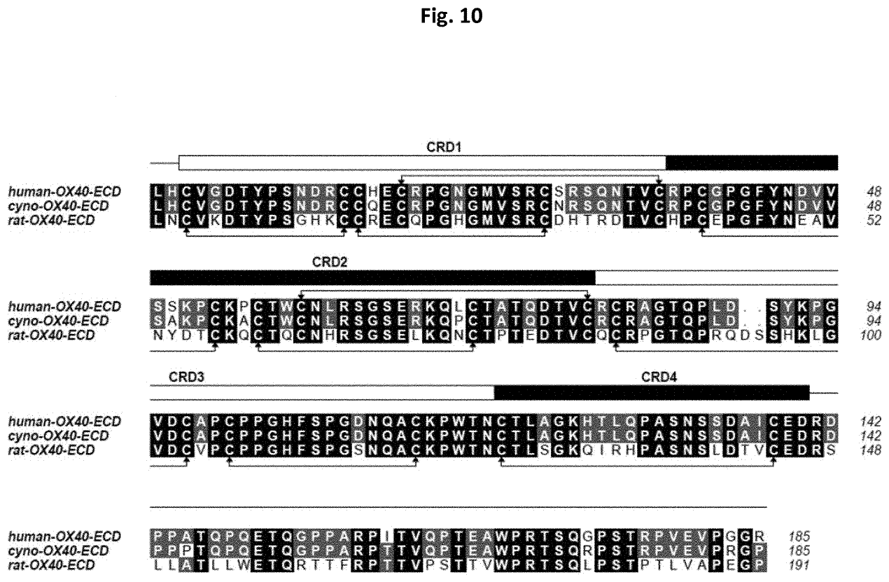

Sequence alignment of human (SEQ ID NO: 1), cynomolgus monkey (SEQ ID NO:122), and rat (SEQ ID NO:121) OX40 extracellular domains was carried out with T-coffee (Notredame C. et al. J Mol Biol, 302 (205-217) 2000) (FIG. 10). CRDs were identified based on disulfide bond patterns in the sequence of OX40. The theoretical sequences of human/rat OX40 chimeras were established by mixing CRDs as follows: human CRD1, CRD2, CRD3 and rat CRD4 (HHHR) (SEQ ID NO: 126); human CRD1, CRD2, rat CRD3, human CRD4 (HHRH) (SEQ ID NO: 127); human CRD1, CRD2, rat CRD3, CRD4 (HHRR) (SEQ ID NO: 128); human CRD1, rat CRD2, CRD3, CRD4 (HRRR) (SEQ ID NO: 129); rat CRD1, CRD2, human CRD3, CRD4 (RRHH) (SEQ ID NO: 130); rat CRD1, human CRD2, rat CRD3, CRD4 (RHRR) (SEQ ID NO: 131). Designed sequences were then gene synthesized by Geneart AG and obtained cDNA were cloned in frame of a human IgG1-Fc region into a modified pcDNA3.1 vector. Proteins were then produced and purified as previously described. These molecules were then used in the previously described ELISA and Biacore experiments to determine the OX40 domains targeted by these antibodies. Results are summarized in table 17.

TABLE 17
Characterization of anti-OX40 antibody domain specificity
Domain specificity of anti-OX40 antibodies

| | CRD1 | CRD2 | CRD3 | CRD4 |
|---|---|---|---|---|
| 2H6 | | | X | |
| 7H11 | X | | | |
| 9B12 | | X | | |
| 11D4 | X | | | |
| 106-222 | | X | | |
| pab1949 | | | X | |
| Tetra-hzG3V9 | | | | X |
| Tetra-hz1D10v1 | | | | X |

Using OX40 chimeras, we found that 7H11 and 11D4 are mainly binding to OX40 CRD1. 9B12 and 106-222 are mainly binding to OX40 CRD2. 2H6 and pAB1949 are mainly binding to CRD3. The hzG3V9 and 1D10v1 dAbs are mainly binding to CRD4. As 1A7 cross-reacts with rat OX40, the epitope of this antibody was not characterized.

### EXAMPLE 10: IN VITRO BIOLOGICAL CHARACTERIZATION OF TETRA-8

### 10.1 Tetra-8 Binds Specifically to OX40

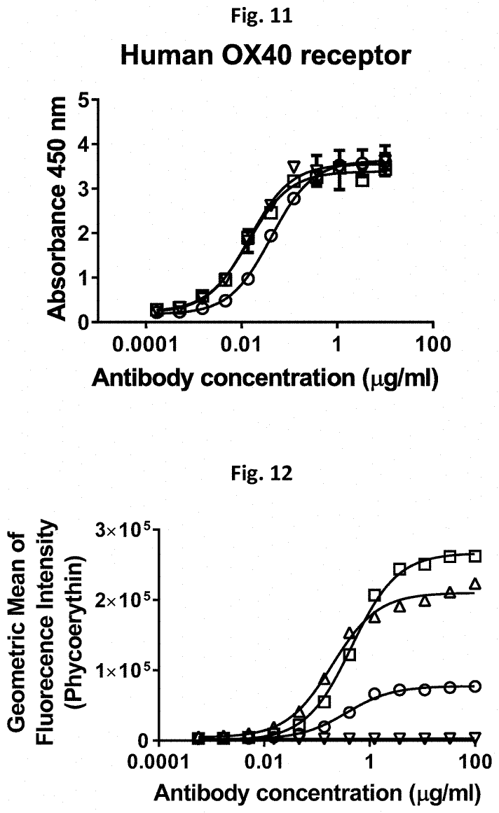

Binding activity of Tetra-8 on soluble OX40 was assessed by direct ELISA, following the method detailed above in example 3. Briefly, Tetra-8 was tested at various concentrations (ranging from 10 to 0.00017 μg/ml) in 96 well-microtiter plates pre-coated overnight with recombinant human OX40 His protein diluted at 2 μg/ml in PBS (see example 1 for the generation of the OX40-his protein). In order to test the two binding units of Tetra-8 individually, 7H11_v8 IgG1 and Tetra-22 molecules were included in the same assay. Tetra-22, as described later, is a control molecule which is composed of an irrelevant IgG1 LALA where the 2H6 ScFvs have been fused to the C-terminus. Results from FIG. 11 show that Tetra-8, 7H11_v8 IgG1 and Tetra-22 molecules recognize recombinant human OX40 protein with equivalent binding profiles.

Binding of Tetra-8 on membrane-bound OX40 was evaluated by flow cytometry using GloResponse™ NFkB luc2/OX40-Jurkat cell line (Promega). In brief, cells were harvested, counted, and plated at 100,000 cells/well in a 96-well round-bottom plate. The plate was centrifuged at 350 g for 3 minutes and the cells were resuspended in 50 μl of FACS buffer (PBS+10% versene+2% FBS) containing various concentrations (ranging from 100 to 0.00056 μg/ml) of either Tetra-8, 7H11_v8 IgG1 or 2H6 IgG1 antibody. Stained cells were incubated for 20 minutes at 4° C., washed twice with FACS buffer at 350 g for 3 min and resuspended in 10001 of an anti-human IgG PE secondary antibody (Thermofischer) diluted in FACS buffer. Cells were then washed twice, and resuspended in 20001 of FACS buffer and samples were acquired on a FACSCalibur instrument (BD Biosciences, Allschwil, Switzerland). The cells were gated based on size on FSC vs SSC and analyzed for PE-geometric mean fluorescence intensity using FlowJo software. As depicted in FIG. 12, Tetra-8, 7H11_v8 IgG1 and 2H6 IgG1 antibodies recognize membrane-bound OX40 expressed on transfected Jurkat cells. The 3 molecules were also directly labeled with AF647 dye as per manufacturer's instructions (Thermofischer) and subsequently evaluated for binding to other cells expressing various levels of OX40. The K_{D} values for all the tested molecules and cell lines are summarized on table 18.

TABLE 18

| | | | Activated | Activated | Activated |
|---|---|---|---|---|---|
| | HPB- | JURKAT | T cells | T cells | T cells |
| | ALL | OX40 | Donor 1 | Donor 2 | Donor 3 |
| 2H6 IgG1 | 15.97 | 17.65 | 3.12 | 8.45 | 8.89 |
| 7H11 IgG1 | 3.57 | 4.47 | 3.58 | 5.40 | 4.80 |
| OX40L-Fc | 5.30 | 5.46 | 9.27 | 19.32 | 13.54 |
| Tetra-8 | 2.47 | 3.62 | 2.03 | 2.28 | 3.40 |

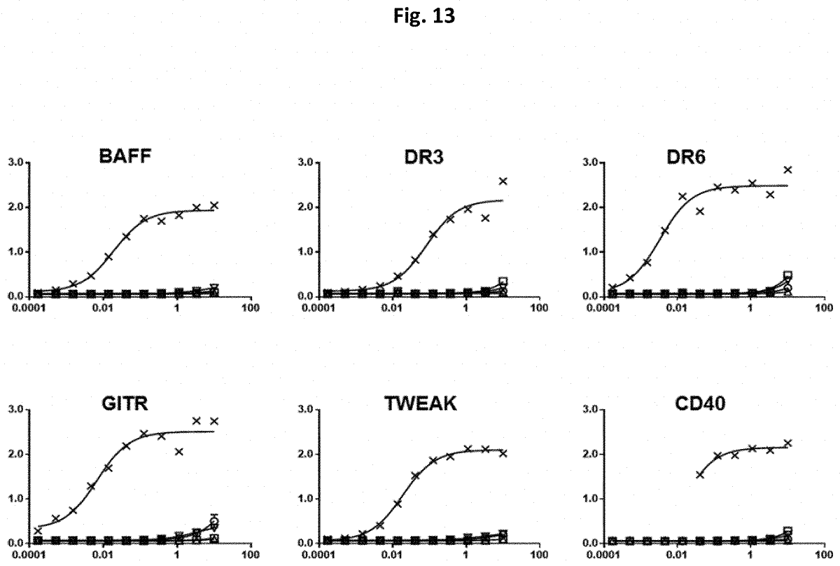

In order to further demonstrate the selective binding of Tetra-8 to OX40, a direct ELISA was performed against other TNFR members. The experiment was conducted following the same protocol than previously described. In this assay, a serial dilution of Tetra-8 (ranging from 10 to 0.0001 μg/ml) was tested against recombinant BAFF, CD40, DR3, DR6, GITR, and TWEAK molecules (R&D). These molecules were all coated at 2 μg/ml in PBS overnight at 4° C. Results from FIG. 13 show that Tetra-8 binds selectively to OX40 molecule, and does not recognize other members of TNFR family, including those which display up to 40% of identity in their amino-acid sequence.

### 10.2 Tetra-8 Binds to Cynomolgus OX40 Via its 2H6 Portion

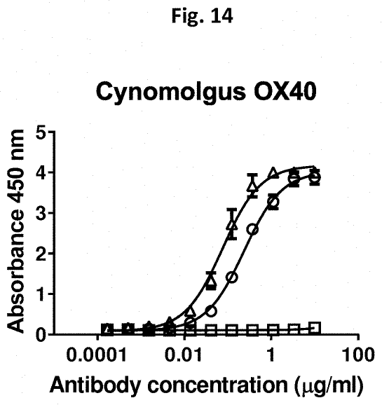

To assess the cross-reactivity of Tetra-8 to cynomolgus OX40, a direct ELISA was performed following the protocol described previously. In this assay, Tetra-8 and Rituximab-2H6 (which is a control molecule equivalent to Tetra-22, in which the irrelevant IgG1 LALA portion is from Rituximab) bind to recombinant cynomolgus OX40 protein, but not 7H11_v8 IgG1 (FIG. 14). These data demonstrate that Tetra-8 recognizes cynomolgus OX40 through its 2H6 portion. Results from binding activities to human and cynomolgus OX40 and EC₅₀ values are summarized in table 19.

TABLE 19

| | | | |
|---|---|---|---|
| Human | cynomolgus | Human | Cynomolgus |
| OX40 | OX40 | OX40 | OX40 |

| | | |
|---|---|---|
| Unit | μg/mL | nM |

| | | | | |
|---|---|---|---|---|
| Tetra-8 | 0.041 | 0.237 | 0.207 | 1.185 |
| 7H11 | 0.014 | NA | 0.093 | NA |
| IgG1 | | | | |
| Tetra-24 | 0.015 | 0.076 | 0.097 | 0.509 |

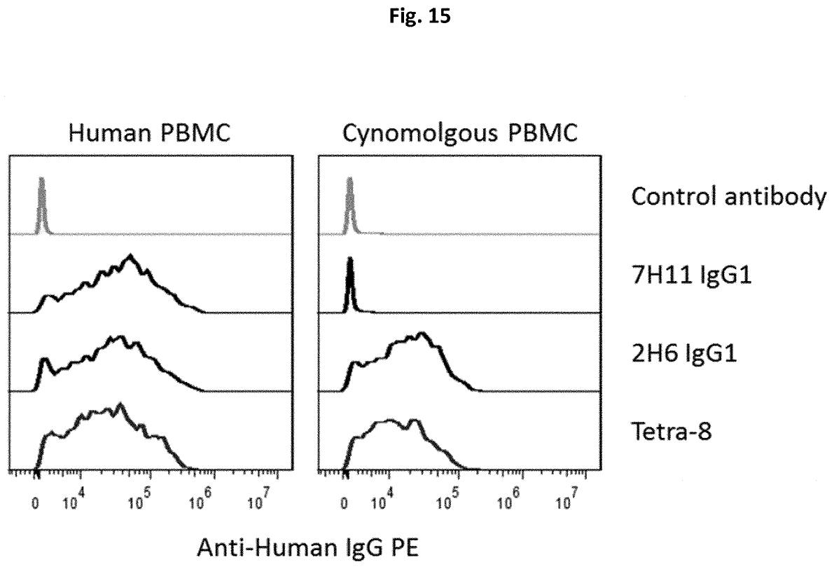

The cross-reactivity of Tetra-8 with cynomolgus OX40 was further demonstrated by flow cytometry. In this procedure, cynomolgus PBMCs from a commercial source (Silabe) were diluted in cRPMI and plated in T-25 flask at 1×10⁶ cells/ml in the presence of 5 μg/ml of PHA at 37° C. in a CO₂ incubator. The same experiment was conducted using human PBMCs isolated as described earlier. Two days later, activated cells were harvested and labeled with anti-human CD4 APC (Thermofischer). A subsequent staining with either Tetra-8, 7H11_v8 IgG1 or 2H6 IgG1 antibody was performed, followed by a detection with an anti-human IgG PE (Thermofischer). Stained cells were washed and resuspended in 100 μl of FACS buffer, and analyzed by flow cytometry using CytExpert (Beckman). The gating strategy consisted in gating on living cells (based on FSC and SSC plots), and CD4 positive cells. Results from FIG. 15 show that Tetra-8, 2H6 IgG1, but not 7H11_v8 IgG1 bind to membrane-bound cynomolgus OX40 expressed on CD4 positive cells. These data confirm that the cynomolgus cross-reactive activity of Tetra-8 is mediated by its 2H6 portion.

### 10.3 Tetra-8 Induces a Significant Activation of the OX40-NFkB Luciferase Reporter Cell Line in an FcγR-Independent Manner

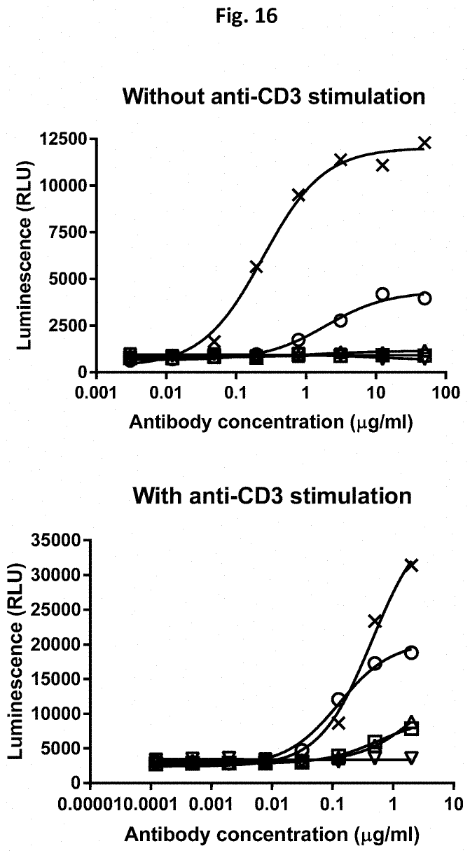

In order to evaluate the potential of Tetra-8 to activate OX40-signaling, a luciferase assay was performed using the GloResponse™ NFkB luc2/OX40-Jurkat cell line expressing OX40, following manufacturer's instructions (Promega). Briefly, Jurkat NFkB were harvested, counted, and resuspended at 2×10⁶ cells/ml in complete RPMI medium (RPMI 1640+10% FBS+1% NEAA+1% NaPyr+hygromycin 500 μg/ml+G418 800 μg/ml). Fifty μl of cells were distributed in a 96-well luminescence plate and incubated at 37° C., 5% CO₂ with 25 μl of either Tetra-8, 7H11 IgG1 LALA or 2H6 IgG1 LALA, serially diluted in the assay medium (RPMI 1640+% FBS). In parallel, these molecules were tested in conditions where a TCR stimulation was applied, by immobilizing 5 μg/ml of anti-CD3 antibody (OKT3 clone) on 96-well luminescence plates. Five hours later, 75 μl of Bio-Glo solution was added to the wells and signals for all the plates were read in a luminescence microplate reader (Synergy HT2-Spectrophotometer, Biotek). The luciferase assay was performed without any crosslinking conditions, that is in the absence of secondary antibody or cells expressing FcγRs. As depicted in FIG. 16, Tetra-8 induces a dose-dependent activation of the OX40 NFkB luciferase reporter Jurkat cell line in two different experimental settings (with or without TCR stimulation), and promotes a higher OX40 signaling than its individual binding units (7H11 IgG1 LALA and 2H6 IgG1 LALA). Importantly, Tetra-8 was further tested in an ADCC assay and did not show, as expected, a significant activity (data not shown). These data highlight important differences between Tetra-8 and other monoclonal OX40 agonist antibodies, which are described to exhibit their activity through Fc [symbol] R-mediated crosslinking.

### 10.4 Tetra-8 Increases T-Cell Allogeneic Activity in MLR Assay

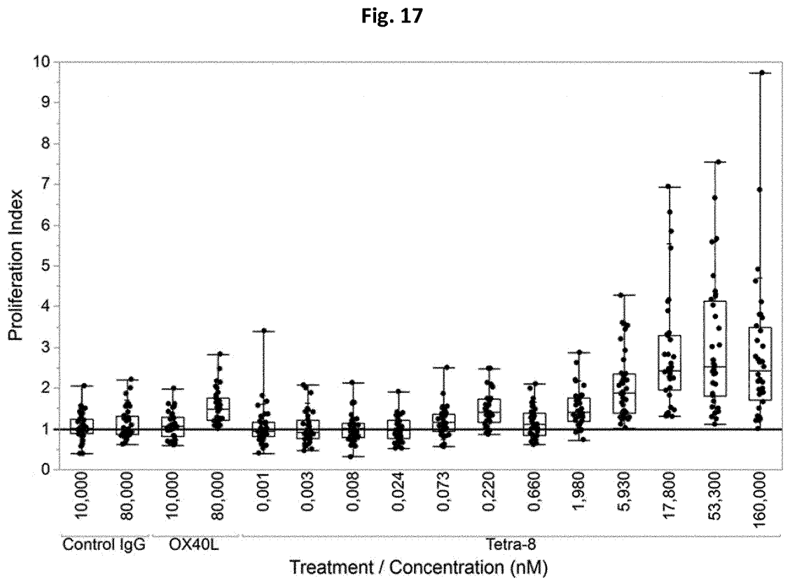

MLR assay is commonly used to evaluate the potential of immunomodulators targeting co-stimulatory molecules, such as OX40, to enhance allogeneic T-cell responses (Keli L. Hippen et al. Blood 2008). In order to evaluate the potential of Tetra-8 to enhance T-cell responses, an allogeneic MLR assay was performed following the protocol described in example 3. In this assay, Tetra-8 was tested at 6 different concentrations (ranging from 160 to 0.001 nM) and OX40L was tested at 80 and 10 nM. Results depicted in FIG. 17 show that Tetra-8 strongly and significantly enhances (by 2 to 3 fold) alloreactive T cell proliferation in the MLR assay. This effect is dose-dependent and even more potent than OX40L as summarized in table 20.

TABLE 20

| | | Mean | Number | | |
|---|---|---|---|---|---|
| | % donor | (fold increase | of | | |
| Molecule | responder | proliferation | tested | | |
| ID | (>2) | vs control) | donors | p-value | Significancy |
| OX40L-Fc | 14% | 1.54 | 37 | <.0001 | *** |
| Tetra-8 | 71% | 2.99 | 38 | <.0001 | *** |
| Tetra-8 | / | / | / | <.0001 | *** |
| compared | | | | | |
| to | | | | | |
| OX40L-Fc | | | | | |

### 10.5 Tetra-8 Induces a Strong Immunostimulatory Effect in a Staphylococcal Enterotoxin B Stimulation Assay

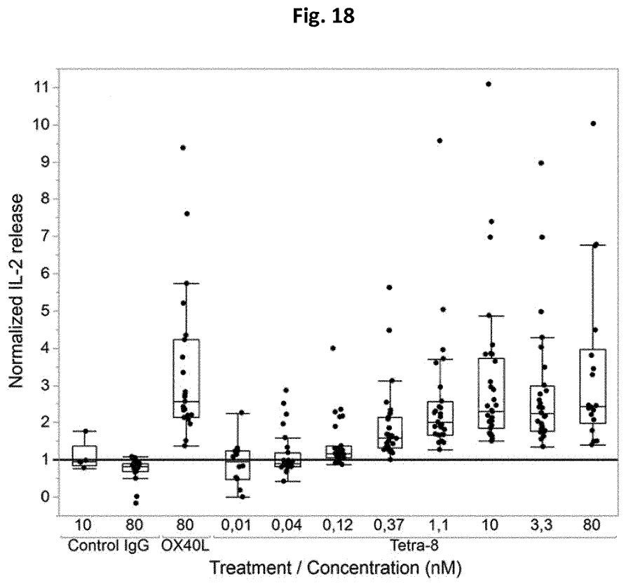

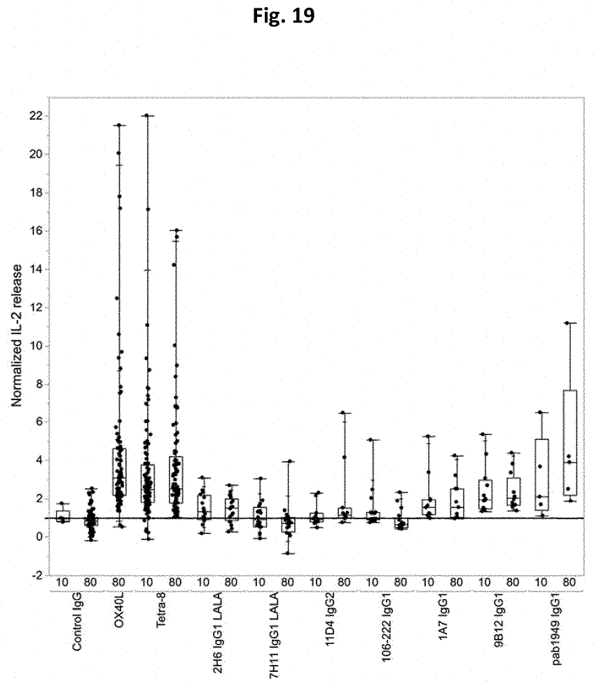

The SEB stimulation assay has also been widely used to evaluate the potential of immunomodulators targeting OX40 and other members of TNFR family to enhance T-cell responses. This assay, which protocol is described in example 8.2, was used to evaluate the functional activity of Tetra-8. Tetra-8 was tested at various concentrations ranging from 80 to 0.01 nM. As shown in FIG. 18, incubation of human PBMCs with Tetra-8 results in a substantial increase in the proliferative activity of T-cells in response to SEB antigen. Tetra-8 was also tested in comparison with other monoclonal anti-OX40 agonists, used at 80 and 10 nM. Results from FIG. 19 demonstrate that 7H11_v8 IgG1, 2H6 IgG1 and other anti-OX40 monoclonal agonistic antibodies tested in the same SEB assay enhance SEB-induced proliferation of T-cells, compared to the isotype control. However, Tetra-8 displays a significantly higher level of agonism compared to all the tested monospecific bivalent anti-OX40 molecules. Overall, results from SEB and MLR assays show that Tetra-8 enhances T-cell responses and displays higher potency than other agonistic anti-OX40 molecules. In contrast to monoclonal agonistic anti-OX40 antibodies which are described to work essentially via FcγR-mediated crosslinking, activity of Tetra-8 is FcγR-independent, as shown previously. Furthermore, testing of 9B12 in SEB assays results in an increase in IL-2 levels but decreased numbers of activated CD4+ T-cells (CD4+CD25+). In contrast, Tetra-8 promotes expansion of this T-cell subset while inducing high levels of IL-2 (data not shown). This strong FcγR-independent agonistic activity of Tetra-8 is probably related to the higher valency and/or its architecture. This hypothesis was further explored in examples 12 and 14.

### EXAMPLE 11: GENERATION OF ALTERNATIVE TETRA-8 ARCHITECTURES

### Molecular Design and Expression

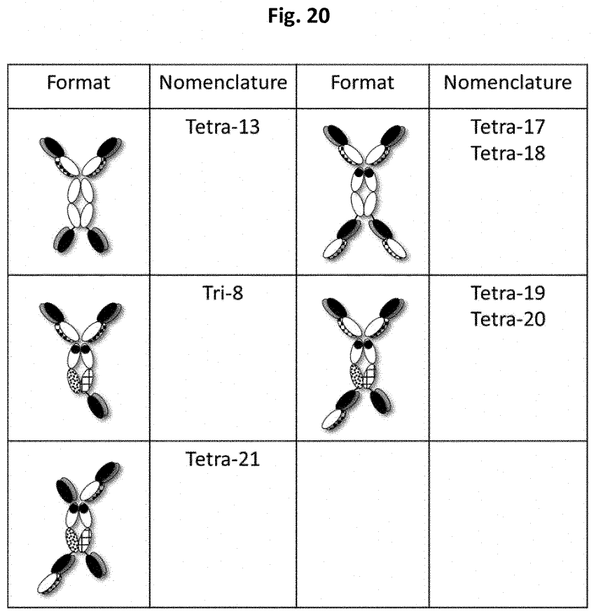

To monitor the effect of FcγR engagement on Tetra-8 biological activity, a modified version of Tetra-8 having wt IgG1 Fc (Tetra-13) was cloned and produced as previously described. Then, to determine the repercussion of Tetra-8 architecture on its agonist properties, several constructs were produced having different binder combination, orientation and valences (FIG. 20).

The Tetra-14 molecule is a tetravalent antibody wherein the 7H11-VH2 N58K-D54E and 7H11 VL1 sequences were formatted as scFv engineered with disulfide bond (gene synthesised by Geneart AG) and further cloned, in the modified pcDNA3.1 vector, in 3′ of the 2H6 IgG1 LALA heavy chain sequence, as previously described for Tetra-8. The 2H6 VL was cloned in frame of the human Kappa constant region (2H6-LC) in the modified pcDNA3.1 vector. The Tetra-14 was produced by co-transfecting vectors coding Tetra-14 HC and 2H6 LC in HEK293-EBNA1 cells (table 21). Cell supernatant was then collected and molecules were purified using protein A affinity purification column as previously described. Then, an additional cation exchange purification step was carried it out to remove covalent multimers contaminants, as previously described for Tetra-8. Therefore, this molecule is composed of the same binders used in Tetra-8 but in reversed orientation.

Tetravalent molecules having the same 4 binders were also designed and gene synthesized by Geneart AG. The Tetra-15 antibody is composed of 2H6 binders while Tetra-16 is composed of 7H11 binders. In this format, 2H6 and 7H11 scFv sequences, engineered with disulfide bond, were fused to 2H6 and 7H11 IgG1 LALA heavy chain sequences, respectively. Tetra-15 and Tetra-16 were produced by transfecting vectors coding for Tetra-15 and Tetra-16 heavy chain with vectors coding 2H6 LC and 7H11 VL1, respectively. Proteins were purified using Protein A chromatography and further polished by cation exchange, as previously described. Therefore, Tetra-15 and Tetra-16 have a format which is similar to Tetra-8 molecule while having 4 related binding arms. Furthermore, the Tetra-17 and Tetra-18 antibodies were designed to have 4 identical binding arms. In the Tetra-17 and 18 format, Fabs are fused in C-terminus of IgG1 heavy chain. The Tetra-17 heavy chain sequence is composed of the 7H11-VH2 N58K-D54E IgG1 heavy chain bearing the LALA mutation where the 7H11-VH2 N58K-D54E-IgG1 CH1 sequence was fused to IgG1 CH3 domain through a short Gly₄Thr (G₄T) linker. Similarly, the Tetra-18 heavy chain is made of the 2H6 IgG1 LALA sequence linked to a sequence coding for 2H6 Fab. Using the same methodology described earlier, vectors coding Tetra-17 and Tetra-18 heavy chains were co-transfected with vectors coding for 7H11 VL1 and 2H6 LC, respectively, in HEK293-EBNA1 cells to produce Tetra-17 and Tetra-18 antibodies (table 21).

Other tetravalent antibodies were designed to combine three identical binders with one unrelated binder and were gene synthesized by Geneart AG. The Tetra-19 antibody is a combination of three 7H11 Fabs with one 2H6 scFv. In this format, heavy chain heterodimerization is required. Therefore, the BEAT technology (Skegro D., et al., J Biol Chem., 292(23):9745-59, June 2017) was used to produce and purify the Fc heterodimer. Two different heavy chains were built and cloned in two different vectors. The first heavy chain (Tetra-19 HC1) comprises, from N to C-terminus, the domain sequences of 7H11-VH2 N58K-D54E, IgG1 CH1, IgG1 hinge, IgG1 CH2 containing the LALA mutation, IgG3 CH3 BEAT (A), G₄T linker and 2H6 scFv engineered with disulfide bond. The second chain (Tetra-19 HC2) is made, from N to C-terminus, of the domain sequences of 7H11-VH2 N58K-D54E, IgG1 CH1, IgG1 hinge, IgG1 CH2 containing the LALA mutation, IgG1 CH3 BEAT (B), G₄T linker, 7H11-VH2 N58K-D54E and IgG1 CH1. The vectors coding for these two different heavy chains were transfected at equimolar ratio with the vector coding for 7H11 VL1 light chain in HEK293-EBNA1 cells using the same protocol that was previously described (table 21). The BEAT technology induces preferential heterodimerization of BEAT(A) and BEAT(B) containing chains. However, some homodimer impurities can still be produced. Nevertheless, the BEAT technology was engineered for heavy chain asymmetric protein A binding which allows efficient purification of the heterodimer from the homodimers contaminants present in the supernatant of expressing cells. Briefly, the clarified supernatant of transfected cells was loaded onto a HiTrap™ MabSelect SuRe™ Protein A column pre-equilibrated in 0.2 M citrate/phosphate buffer, pH 6, and operated on an ÄKTA™ purifier chromatography system (both from GE Healthcare Europe GmbH) at a flow rate of 1 ml/min. Running buffer was 0.2 M citrate/phosphate buffer, pH 6. Wash buffer was 0.2 M citrate/phosphate buffer, pH 5. Heterodimer elution was performed using 20 mM sodium acetate buffer, pH 4.1.

Elution was followed by absorbance reading at 280 nm; relevant fractions containing the heterodimer, Tetra-19, were pooled and neutralized with 0.1 volume of 1 M TrisHCl, pH 8. An additional cation exchange purification step was then performed to remove covalent multimers. The Tetra-20 antibody, which is a combination of three 2H6 Fabs with one 7H11 scFv, was produced and purified using the same protocol. The Tetra-20 HC1 comprises, from N to C-terminus, the domain sequences of 2H6 VH, IgG1 CH1, IgG1 hinge, IgG1 CH2 containing the LALA mutation, IgG3 CH3 BEAT (A), G₄T linker and 7H11 scFv engineered with disulfide bond. The Tetra-20 HC2 is made, from N to C-terminus, of the domain sequences of 2H6 VH, IgG1 CH1, IgG1 hinge, IgG1 CH2 containing the LALA mutation, IgG1 CH3 BEAT (B), G₄T linker, 2H6 VH and IgG1 CH1. These two chains were co-expressed with 2H6 light chain to produce Tetra-20 (table 21) which was purified using differential protein A and cation exchange chromatography.

The Tetra-21 antibody was designed to contain four antibody binding domains with one 2H6 scFv and one 7H11 Fab fused in N-terminus of the Fc-region and one 2H6 scFv and one 7H11 Fab fused in C-terminus of the same Fc-region. This heterodimer was produced, as previously described, by co-expressing Tetra-19 HC1 with Tetra-21 HC2 and 7H11 VL1 light chain (table 21). The Tetra-21 HC2 was built to contain, from N to C-terminus, the 2H6 scFv linked to the IgG1 CH2 containing the LALA mutation followed by the IgG1 CH3 BEAT (B), the G4T linker, the 7H11-VH2 N58K-D54E and the IgG1 CH1. This chain was synthesized by Geneart AG and cloned into the modified pcDNA3.1 vector. Tetra-21 was then purified using differential protein A chromatography followed by an additional cation exchange purification step, as previously described.

The Tetra-22 was designed to combine trastuzumab Fab with 2H6 scFv fused in C-terminus of the Fc region as a control of 2H6 scFv agonist activity alone. The Tetra-22 HC was built to contain, from N to C-terminus, the trastuzumab VH, the IgG1 CH1, the IgG1 hinge region, the IgG1 CH2 containing the LALA mutation, the IgG1 CH3, the G4T linker and the 2H6 scFv disulfide bond engineered. This heavy chain was gene synthesized by Geneart AG as well as the trastuzumab VL (Tetra-22 LC), both chains were then cloned into modified pcDNA3.1 vector. Tetra-22 was then produced and purified as previously described for Tetra-8 (table 21). Similarly to the C-terminal fusion of 2H6 scFv to trastuzumab IgG1 LALA, the rituximab was used as an irrelevant binder and a tetravalent molecule rituximab-2H6 was produced using Tetra-8 and Tetra-22 architecture as templates.

Finally, the Tri-8 molecule was generated to produce a trivalent molecule having two 7H11 Fab and only one 2H6 scFv fused in C-terminus. This heterodimer was made by combining the Tetra-19 HC1 with the Tri-8 HC2 and the 7H11 VL1 light chain (table 21). The Tri-8 HC2 is composed, from N to C-terminus, of the 7H11-VH2 N58K-D54E, the IgG1 CH1, the IgG1 CH2 containing the LALA mutation followed by the IgG1 CH3 BEAT (B). This chain was gene synthesized by Geneart AG and cloned into the modified pcDNA3.1 vector. Tri-8 was then produced and purified as previously described for Tetra-19, 20 and 21.

TABLE 21
Combination of heavy and light chains for
the production of tetravalent and trivalent antibody.
Heavy and light chains
used for tetravalent antibody production

| Tetra- | | | |
|---|---|---|---|
| valent | Heavy chain 1 | Heavy chain 2 | Light chain |
| Tetra-8 | Tetra-8 HC | — | Humanized 7H11-VL1 |
| | (SEQ ID: 45) | | (SEQ ID: 16) |
| Tetra-13 | Tetra-13 HC | — | Humanized 7H11-VL1 |
| | (SEQ ID: 132) | | (SEQ ID: 16) |
| Tetra-14 | Tetra-14 HC | — | 2H6 LC |
| | (SEQ ID: 133) | | (SEQ ID: 116) |
| Tetra-15 | Tetra-15 HC | — | 2H6 LC |
| | (SEQ ID: 134) | | (SEQ ID: 116) |
| Tetra-16 | Tetra-16 HC | — | Humanized 7H11-VL1 |
| | (SEQ ID: 135) | | (SEQ ID: 16) |
| Tetra-17 | Tetra-17 HC | — | Humanized 7H11-VL1 |
| | (SEQ ID: 136) | | (SEQ ID: 16) |
| Tetra-18 | Tetra-18 HC | — | 2H6 LC |
| | (SEQ ID: 137) | | (SEQ ID: 116) |
| Tetra-19 | Tetra-19 HC1 | Tetra-19 HC2 | ID: Humanized 7H11-VL1 |
| | (SEQ ID: 138) | (SEQ ID: 139) | (SEQ16) |
| Tetra-20 | Tetra-20 HC1 | Tetra-20 HC2 | 2H6 LC |
| | (SEQ ID: 140) | (SEQ ID: 141) | (SEQ ID: 116) |
| Tetra-21 | Tetra-19 HC1 | Tetra-21 HC2 | Humanized 7H11-VL1 |
| | (SEQ ID: 138) | (SEQ ID: 142) | (SEQ ID: 16) |
| Tetra-22 | Tetra-22 HC | — | Tetra-22 LC |
| | (SEQ ID: 143) | | (SEQ ID: 144) |
| Tri-8 | Tetra-19 HC1 | Tri-8 HC2 | Humanized 7H11-VL1 |
| | (SEQ ID: 138) | (SEQ ID: 145) | (SEQ ID: 16) |

### Characterization of Antibodies Having Alternative Tetra-8 Architecture

Molecules were then further characterized by Biacore using the method described earlier. Proteins were digested using FabALACTICA and the Fc-fused C-terminal binders obtained after proteolysis were purified using CaptureSelect™ FcXL. Obtained material were then used to study C-terminal binders potency by Biacore.

The affinity of the 7H11 scFv fused in C-terminus of the Tetra-14 molecule for OX40 was measured using the same approach described for the measurement of 2H6 scFv affinity for OX40 in Tetra-8. An affinity of 19 nM was determined (table 22), indicating that Tetra-14 binding arms fused in C-terminus are functional although a 2-fold decrease of affinity of 7H11 was measured

TABLE 22
Characterization of 7H11 and 2H6 affinity when fused in N or C-terminus.
Affinity of 2H6 and 7H11 to human OX40 depending on their orientation

| | |
|---|---|
| N-ter | C-ter |

| | | | | |
|---|---|---|---|---|
| 2H6 | 27 | nM | 60 | nM |
| 7H11-VH2 N58K-D54E | 9 | nM | 19 | nM |

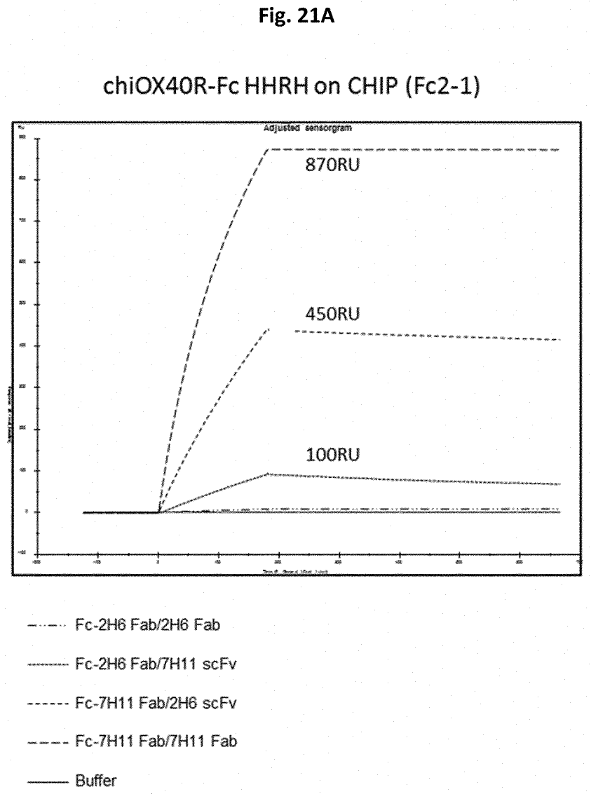

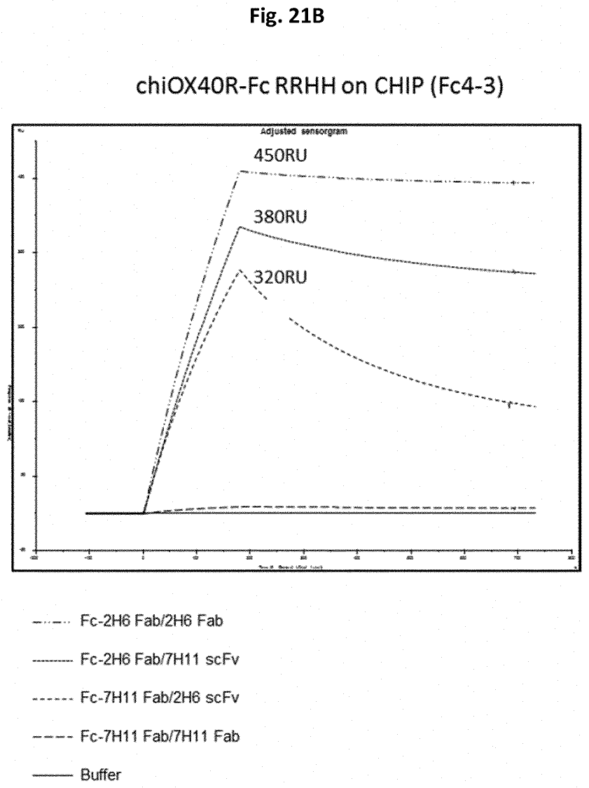

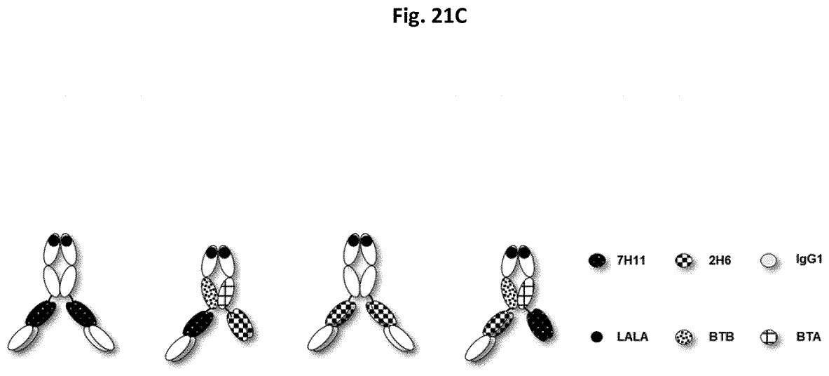

Tetra-17, Tetra-18, Tetra-19 and Tetra-20 were also digested to be studied by Biacore using OX40 chimeras. chiOX40R HHRH-Fc and chiOX40R RRHH-Fc were immobilized on the previously activated CM5 sensor chip (3000 RU) by injecting them to flow path 2 and 4 respectively, to reach 3000 RUs for both molecules. Then, the purified digestion products of Tetra-17, Tetra-18, Tetra-19 and Tetra-20 were used as analytes and injected on the 4 flow-paths (flow-path 1 and 3 being used as references) at a concentration of 200 nM and a flow rate of 30 uL/min for 240 seconds. A second injection was then performed using HBS-EP buffer for 3 min followed by 5 min of dissociation. Regeneration between the injections was done using glycine pH1.5 buffer for 1 min (FIG. 21 and table 23). 7H11 binder is specific of OX40 CRD1, therefore it can only bind to chiOX40R HHRH-Fc but not chiOX40R RRHH-Fc (table 23). In these settings, we observed that the binding of 7H11 to chiOX40R HHRH-Fc when it is fused in C-terminus as a Fab is approximately 4-fold better than in scFv format (comparison of Fc-7H11Fab/2H6 scFv with Fc-7H11 scFv/2H6 fab, 450 response units (RU) versus 100 RUs, respectively) which indicates that 7H11 is functional regardless of its format. We also determined that, in bivalent format, the binding of 7H11 Fab to OX40 CRD1 was 2-fold better than in monovalent format (comparison of Fc-7H11Fab/7H11 Fab with Fc-7H11 Fab/2H6 scFv, 870 RUs versus 450 RUs, respectively), suggesting that the two Fabs fused in C-terminus are both functional and could potentially co-engage two OX40 molecules. 2H6 binder is specific of OX40 CRD3, therefore it can only bind to chiOX40R RRHH-Fc but not chiOX40R HHRH-Fc. We also observed that the binding to chiOX40R RRHH-Fc of 2H6 fused in C-terminus as a Fab is better than in scFv format (comparison of Fc-7H11Fab/2H6 scFv with Fc-7H11 scFv/2H6 fab) confirming that 2H6 is functional when formatted as Fab or scFv. We also observed that, in bivalent format, the binding of 2H6 Fab to OX40 CRD3 was better than in monovalent format (comparison of Fc-2H6 Fab/2H6 Fab with Fc-7H11 scFv/2H6 Fab), suggesting that the 2H6 binding units fused in C-terminus are both functional and could potentially co-engage two OX40 molecules.

TABLE 23
Characterization of 7H11 and 2H6 binding when
fused in C-terminus with different valence and/or format.
Binding of C-terminal 2H6 and 7H11 to CRD1 and CRD3 depending on
their valence and format expressed as number of response units

| | chiOX40R HHRH-Fc | | chiOX40R RRHH-Fc | |
|---|---|---|---|---|
| Fc-7H11 Fab/7H11 Fab | 870 | RU | 0 | RU |
| Fc-7H11 Fab/2H6 scFv | 450 | RU | 320 | RU |
| Fc-7H11 scFv/2H6 Fab | 100 | RU | 380 | RU |
| Fc-2H6 Fab/2H6fab | 0 | RU | 450 | RU |

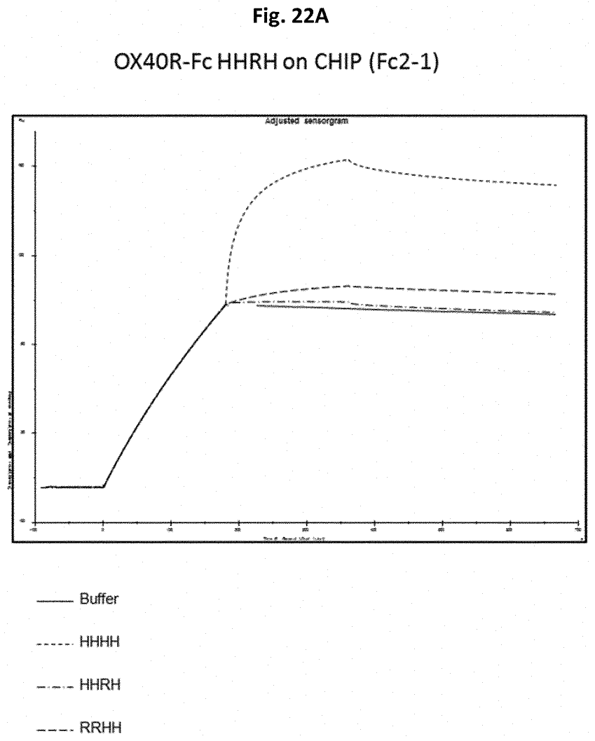

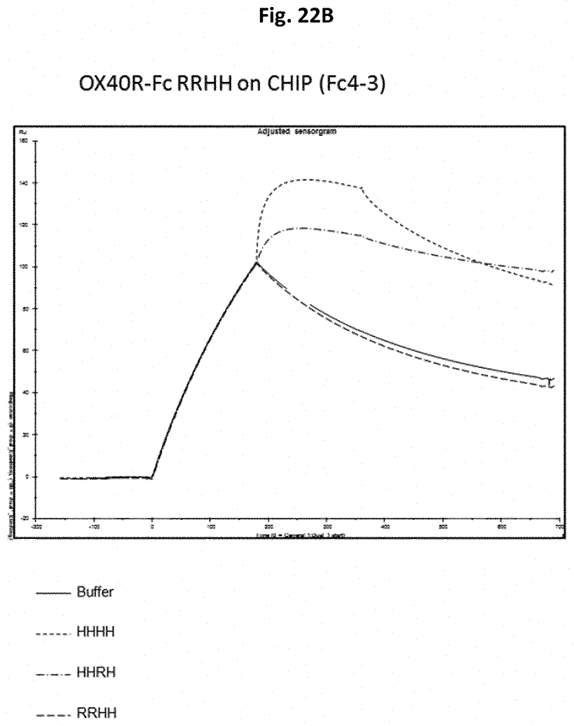

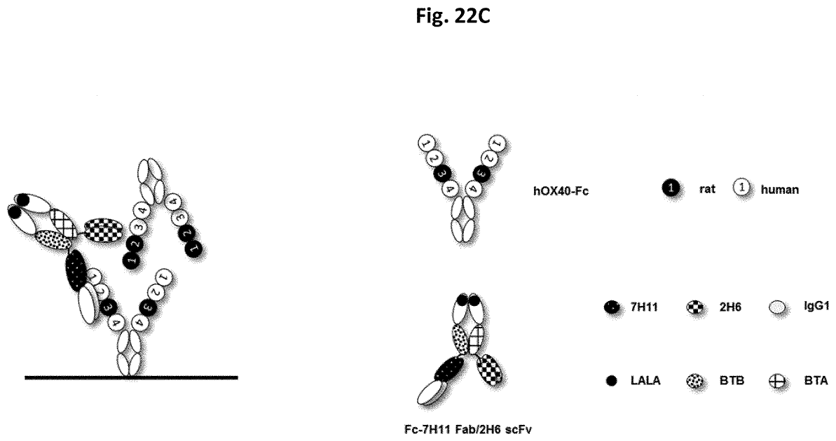

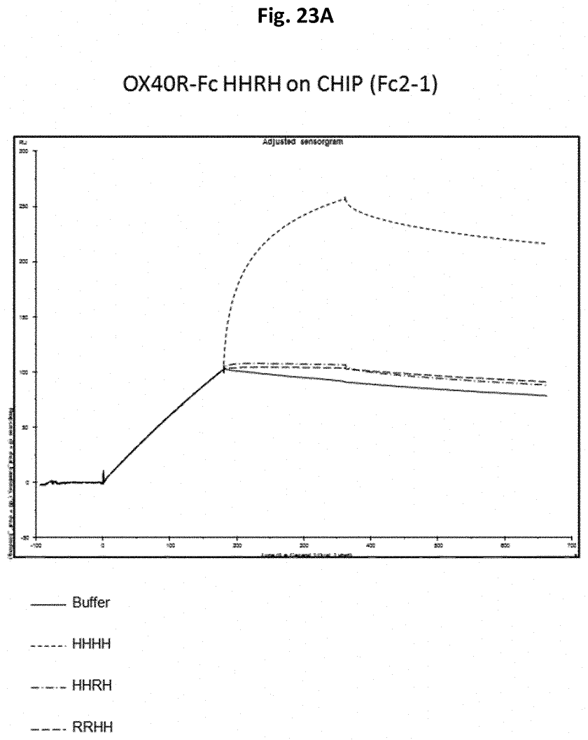

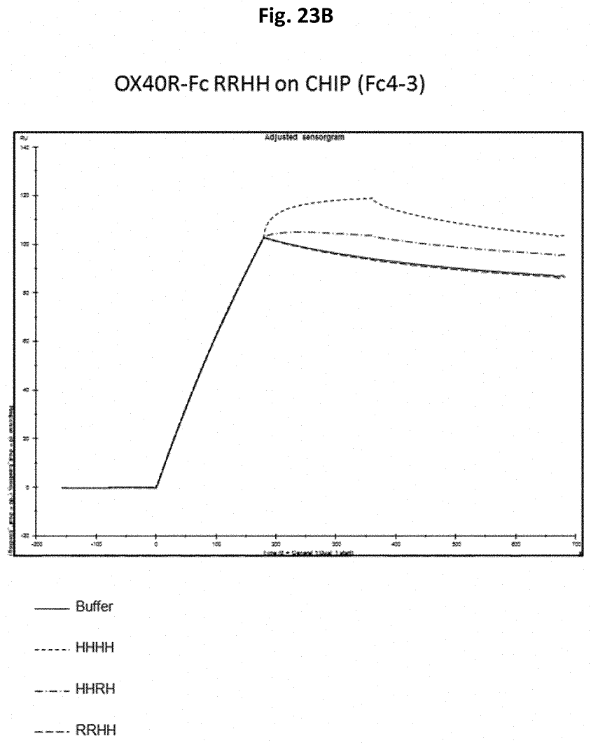

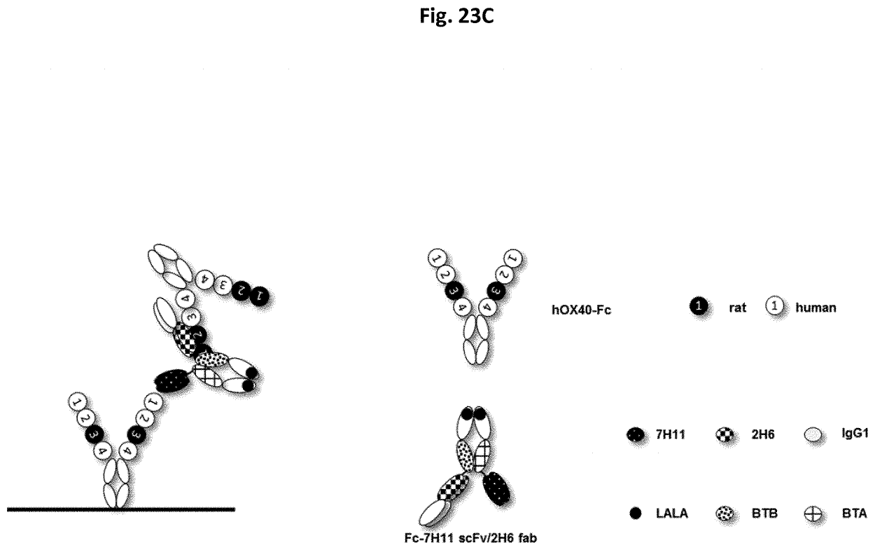

To clearly demonstrate co-binding events, we slightly modified the Biacore set-up described earlier by only using the Fc-7H11 Fab/2H6-scFv or the Fc-7H11 scFv/2H6 Fab as analytes for the first injection and by injecting the buffer, human OX40-Fc, chiOX40R HHRH-Fc (7H11 specific) and chiOX40R RRHH-Fc (2H6 specific) chimeras at 400 nM for 3 min followed by 5 min of dissociation for the second injection step. Regeneration between the injections was performed using glycine pH1.5 buffer for 1 min (FIGS. 22 and 23). Using these approaches co-binding events could be monitored. Effectively, the Fc-7H11 Fab/2H6 scFv portion, when it is captured on the CHIP through the binding of 7H11 to OX40 CRD1, can still interact with human OX40-Fc but also to chiOX40R RRHH-Fc (2H6 specific) (FIG. 22a). Similarly, Fc-7H11 Fab/2H6 scFv portion which is captured on the CHIP via 2H6 binding to OX40 CRD3 can interact human OX40 and chiOX40R OX40 HHRH-Fc (7H11 specific) (FIG. 22b). Similar results were obtained with Fc-7H11 scFv/2H6 Fab (FIGS. 23a and 23b). Taken together, these data indicate that 7H11 or 2H6 binding units fused in C-terminus are functional, regardless of their Fab or scFv formats, and that they can co-engage two different OX40 units at the same time.

### EXAMPLE 13: THE AGONISTIC ACTIVITY OF TETRA-8 IS RELATED TO ITS ARCHITECTURE AND VALENCY

Tetra-8, which exhibits four binding units, displays a higher agonistic activity compared to monoclonal bivalent 7H11_v8 IgG1 or 2H6 IgG1, as shown in example 10. In order to evaluate the contribution of the architecture of Tetra-8 in its biological functions, several variants of Tetra-8 displaying different architectures and valencies were generated and tested in an SEB assay. These molecules are listed in FIG. 20. As shown in FIG. 24, Tetra-8, which is composed of 4 binding portions derived from 2 different clones, triggers a higher agonistic activity than molecules composed of i) 2 binding portions derived from one clone (either 7H11 or 2H6) ii) 3 binding portions derived from 2 different clones (Tri-8) iii) quadrivalent molecules composed of 4 similar binding portions (Tetra-15 and Tetra-16). Two other quadrivalent anti-OX40 variants molecules with different architectures than Tetra-8 were tested in the same assay: Tetra-21 and Tetra-14. These two molecules are composed of the same OX40 binding portions than Tetra-8 (derived from 7H11 and 2H6 clones) but with different orientations. As shown in FIG. 24, both quadrivalent molecules exhibit weak agonistic potentials compared to Tetra-8 antibody in SEB assays. Also, Tetra-8 induces higher IL-2 levels than the combination of 7H11 and 2H6 (7H11 IgG1 LALA+ Tetra-22), as summarized in table 24, which shows that the presence of the four OX40-binding units in the same molecule is key in driving Tetra-8 activity. Taken together, results from FIG. 24 demonstrate that the strong agonistic property of Tetra-8 is related to its quadrivalency and to its architecture.

TABLE 24

| | | | | Is treatment | | Is treatment | |
|---|---|---|---|---|---|---|---|
| | % donor | | Number | X different | | X different | |
| | responder | | of tested | from No | | from | |
| Molecule ID | (>2) | Mean | donors | Treatment | Significancy | Tetra-8 | Significancy |
| Control IgG | 5% | 0.88 | 85 | 0.0263 | * | <.0001 | *** |
| OX40L | 83% | 4.26 | 90 | <.0001 | *** | 0.0013 | ** |
| Tetra-8 | 68% | 3.61 | 84 | <.0001 | *** | / | / |
| 7H11 IgG1 LALA + | 0% | 1.02 | 10 | 0.9098 | NS | 0.0004 | *** |
| Tetra-22 | | | | | | | |
| 2H6 IgG1 LALA | 25% | 1.45 | 16 | 0.0239 | * | 0.0003 | *** |
| 7H11 IgG1 LALA | 6% | 0.81 | 16 | 0.4646 | NS | 0.0001 | *** |
| Tetra-13 | 64% | 2.60 | 11 | 0.0079 | ** | 0.0473 | * |
| Tetra-14 | 40% | 1.65 | 10 | 0.0373 | * | 0.0064 | ** |
| Tetra-15 | 0% | 0.93 | 6 | 0.406 | NS | 0.0083 | ** |
| Tetra-16 | 33% | 1.72 | 6 | 0.0932 | NS | 0.0026 | ** |
| Tetra-17 | 8% | 1.05 | 12 | 0.8024 | NS | 0.0088 | ** |
| Tetra-18 | 0% | 0.88 | 12 | 0.187 | NS | 0.0124 | * |
| Tetra-19 | 0% | 1.00 | 12 | 0.9546 | NS | 0.0006 | *** |
| Tetra-20 | 0% | 1.14 | 12 | 0.2581 | NS | 0.0009 | *** |
| Tetra-21 | 8% | 1.32 | 12 | 0.0349 | * | 0.001 | *** |
| Tetra-22 | 0% | 1.00 | 15 | 0.987 | NS | <.0001 | *** |
| Tetra-23 | 33% | 1.66 | 12 | 0.0287 | * | <.0001 | *** |
| Tri-8 | 0% | 0.96 | 6 | 0.6304 | NS | 0.0045 | ** |
| 106-222 IgG1 | 6% | 0.86 | 17 | 0.3101 | NS | 0.0036 | ** |
| 11D4 IgG2 | 18% | 1.87 | 11 | 0.1372 | NS | 0.0261 | * |
| 1A7 IgG1 | 36% | 1.93 | 11 | 0.0178 | * | 0.0911 | NS |
| 9B12 IgG1 | 50% | 2.36 | 12 | 0.0005 | *** | 0.0198 | * |
| pab1949 IgG1 | 80% | 4.74 | 5 | 0.0884 | NS | 0.6301 | NS |
| Tetra-hz1D10v1 | 50% | 1.89 | 12 | 0.0022 | ** | 0.0114 | * |
| Tetra-hzG3V9 | 50% | 1.93 | 12 | 0.006 | ** | 0.0068 | ** |
| Hexa-hz1D10v1 | 0% | 1.14 | 12 | 0.4894 | NS | 0.0025 | ** |
| Hexa-hzG3V9 | 33% | 1.82 | 12 | 0.0033 | ** | 0.0053 | ** |
| 106-222_1949 | 9% | 0.84 | 11 | 0.3347 | NS | 0.0068 | ** |
| 106-222_2H6_8 | 9% | 1.30 | 11 | 0.1412 | NS | 0.0002 | *** |
| 106-222_hzG3v9 | 0% | 0.51 | 11 | 0.0018 | ** | 0.0115 | * |
| 11D4_1949 | 100% | 11.86 | 11 | 0.0164 | * | 0.0275 | * |
| 11D4_hzG3v9 | 100% | 22.65 | 11 | 0.0557 | NS | 0.063 | NS |
| 1A7_1949 | 9% | 1.43 | 11 | 0.2819 | NS | 0.0054 | ** |
| 1A7_2H6_8 | 0% | 0.79 | 11 | 0.0281 | * | 0.0002 | *** |
| 2H6_hzG3v9_8 | 100% | 48.39 | 11 | 0.087 | NS | 0.0935 | NS |
| 7H11_1949_8 | 100% | 11.89 | 11 | 0.024 | * | 0.0504 | NS |
| 7H11_9B12_8 | 73% | 6.47 | 11 | 0.0368 | * | 0.0844 | NS |
| 7H11_hzG3v9_8 | 100% | 30.34 | 11 | 0.0798 | NS | 0.0894 | NS |
| 9B12_1949 | 18% | 1.60 | 11 | 0.119 | NS | 0.0081 | ** |
| 9B12_hzG3v9 | 91% | 12.42 | 11 | 0.0154 | * | 0.0169 | * |

### EXAMPLE 13 TETRAVALENT ANTIBODY TARGETING OX40 DOMAINS

The Tetra-8 agonist antibody engages OX40 CRD1 and CRD3 through its 7h11 and 2H6 binding units, respectively. The Tetra-8 tetravalent architecture, consisting in disulfide engineered scFv fused to the C-terminal part of an IgG1 LALA heavy chain, seems to be optimum for its agonist activity. In addition, the data obtained with antibodies sharing similar binding units with Tetra-8 but having different architectures also suggest that the N-ter Fab portion has to target membrane distal OX40 domain while the C-terminal scFv should interact with membrane proximal OX40 domain. The 2H6, 7H11, 9B12, 11D4, 1A7, 106-222, pab1949 and hzG3V9 OX40 binding units were used to determine whether other OX40 binders combined in a tetravalent format could agonize OX40. Anti-OX40 antibody sequences were assembled using the Tetra-8 specific format as template (i.e. composed of VH, IgG1 CH1, IgG1-hinge, IgG1 CH2 LALA, IgG1 CH3, linker, disulfide engineered scFv or dAb from N to C terminus). In N-terminus, the VHs of 7H11, 11D4, 1A7, 9B12, 106-222 and 2H6 were selected while in C-terminus, the scFvs of 9B12, 2H6, pab1949 and the hzG3v9 dAb were chosen. Binder combination were designed to explore different OX40 epitope engagement by tetravalent molecules (table 25). Then, cDNA encoding the designed heavy chains, 1A7_2H6_8, 106-222_2H6_8, 7 H11_1949_8, 11 D4_1949, 1A7_1949, 9B12_1949, 106-222_1949, 7 H11_9B12_8, 7 H11_hzG3v9_8, 11 D4_hzG3v9, 106-222_hzG3v9, 9B12_hzG3v9 and 2H6_hzG3v9_8 were gene synthesized by GeneArt before cloning in a modified pCDNA3.1 vector, as previously described. For the production of these tetravalent antibodies, heavy chains were co-transfected with their respective light chains (table 26) in HEK293-EBNA1 cells and the supernatants were collected before protein A purification. Then, an additional cation exchange purification step was used to remove covalent multimers formed with tetravalent molecules having disulfide engineered scFv fused in C-terminus.

TABLE 25
Combination of OX40 binders in
tetravalent antibody format based on their epitope
OX40 Binders combined in tetravalent format

| | | | |
|---|---|---|---|
| CRD2 | CRD3 | CRD4 | C-ter |

| | | | |
|---|---|---|---|
| CRD1 | 7H11/9B12 | 7H11/pab1949 | 7H11/hzG3v9 |
| | | 11D4/pab1949 | 11D4/hzG3v9 |
| CRD2 | | 106-222/2H6 | 106-222/ |
| | | | hzG3v9 |
| | | 106-222/pab1949 | 9B12/hzG3v9 |
| | | 9B12/pab1949 | |
| CRD3 | | | 2H6/hzG3v9 |
| N-ter | | | |

TABLE 26
Combination of heavy and light chains for tetravalent antibody production
Heavy and light chains used for tetravalent antibody production

| Tetravalent | Heavy chain | Light chain |
|---|---|---|
| 1A7_2H6_8 | 1A7_2H6_8 HC | 1A7 LC |
| | (SEQ ID: 146) | (SEQ ID: 108) |
| 106-222_2H6_8 | 106-222_2H6_8 HC | 106-222 LC |
| | (SEQ ID: 147) | (SEQ ID: 106) |
| 7H11_1949_8 | 7H11_1949_8 HC | Humanized 7H11-VL1 |
| | (SEQ ID: 148) | (SEQ ID: 16) |
| 11D4_1949 | 11D4_1949 HC | 11D4 LC |
| | (SEQ ID: 149) | (SEQ ID: 152) |
| 1A7_1949 | 1A7_1949 HC | 1A7 LC |
| | (SEQ ID: 150) | (SEQ ID: 108) |
| 9B12_1949 | 9B12_1949 HC | 9B12 LC |
| | (SEQ ID: 1511) | (SEQ ID: 104) |
| 106-222_1949 | 106-222_1949 HC | 106-222 LC |
| | (SEQ ID: 152) | (SEQ ID: 106) |
| 7H11_9B12_8 | 7H11_9B12_8 HC | Humanized 7H11-VL1 |
| | (SEQ ID: 153) | (SEQ ID: 16) |
| 7H11_hzG3v9_8 | 7H11_hzG3v9_8 HC | Humanized 7H11-VL1 |
| | (SEQ ID: 15554444) | (SEQ ID: 16) |
| 11D4_hzG3v9 | 11D4_hzG3v9 HC | 11D4 LC |
| | (SEQ ID: 1555) | (SEQ ID: 102) |
| 106- | 106-222_hzG3v9 HC | 106-222 LC |
| 222_hzG3v9 | (SEQ ID: 156) | (SEQ ID: 106) |
| 9B12_hzG3v9 | 9B12_hzG3v9 HC | 9B12 LC |
| | (SEQ ID: 157) | (SEQ ID: 104) |
| 2H6_hzG3v9_8 | 2H6_hzG3v9_8 HC | 2H6 LC |
| | (SEQ ID: 158) | (SEQ ID: 116) |

Characterization of pab1949 Affinity when Fused in N or C-Terminus.

The affinity of the pab1949 scFv fused in C-terminus was measured using the same approach described for the measurement of 2H6 scFv affinity for OX40 in Tetra-8. An affinity of 460 nM was determined (table 27). To determine the affinity of pab1949 IgG1 for OX40, approximately 600 RUs of this antibody was captured on CM5 chip where anti-human IgG Fc was previously immobilized. Dilution series of hsOX40_CRD_Avi_His (SEQ ID NO: 159) were then injected. In this format, an affinity of 304 nM was measured for pab1949.

TABLE 27
Affinity of pab1949 as IgG1 or scFv fused in C-terminus.
Affinity of pab1949 to human OX40

| | IgG1 | scFv fused in C-terminus |
|---|---|---|
| pab1949 | 304 nM | 460 nM |

### EXAMPLE 14: ENGAGEMENT OF MULTIPLE EPITOPES OF OX40 INCREASES ITS AGONISTIC POTENTIAL

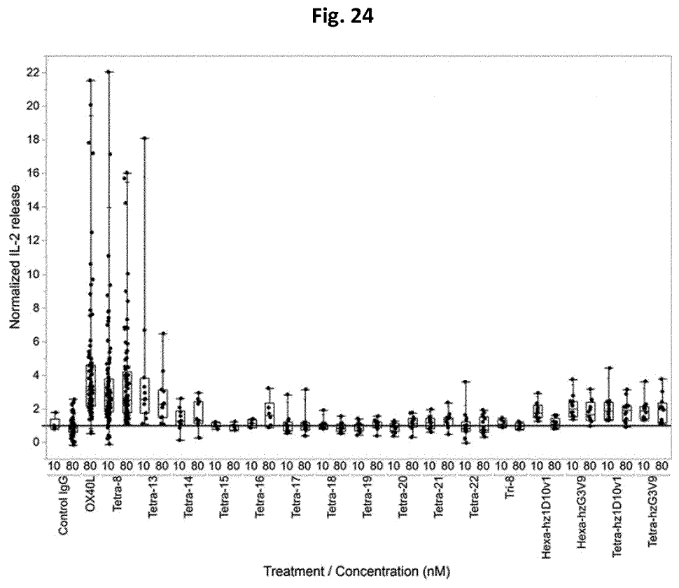

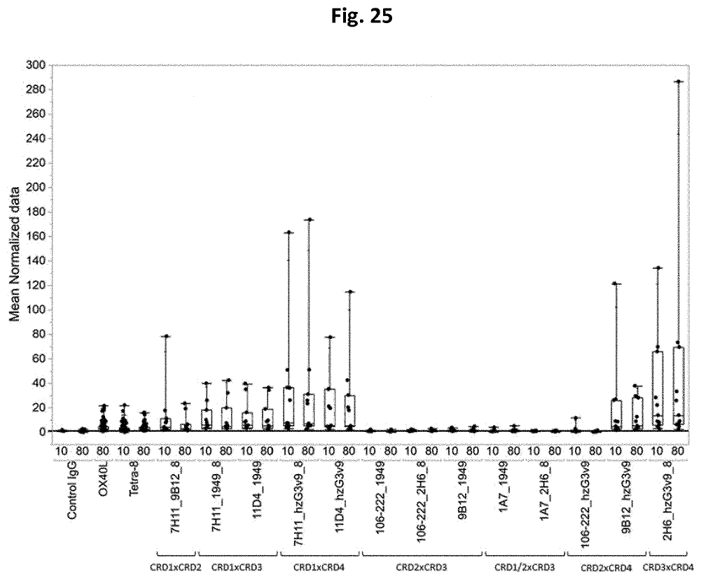

The functional differences observed between Tetra-8 and Tetra-16 or between Tetra-8 and Tetra-15 strongly suggest that molecules composed of multiple binding units targeting different epitopes of OX40 show a higher agonistic potential than multivalent monospecific molecules. In order to validate this hypothesis, molecules composed of binding units recognizing different OX40 epitopes were generated and tested in an SEB assay. As depicted in FIG. 25, Tetra-8, 7H11_1949_8 and 11D4_1949 molecules, which are both quadrivalent molecules composed of binding units specific for domains 1 and 3 of OX40, exhibit higher agonism than Tetra-15 or Tetra-16 quadrivalent monospecific antibodies. Similarly, quadrivalent 7H11_hzG3v9_8 and 11D4_hzG3v9, which target domains 1 and 4 of OX40, trigger more potent agonistic activity than their bivalent counterparts. These data show that Tetra-8 exhibits a potent OX40 agonistic activity via a multivalent and bi-epitopic targeting of membrane distal and proximal domains of OX40. Furthermore, the combination of 7H11 IgG1 LALA+Tetra-22 induces higher IL-2 levels compared to individual molecules but does not recapitulate the levels of IL-2 induced by Tetra-8, as shown on Table 24. This demonstrates that bi-epitopic targeting of OX40 mediated by quadrivalent Tetra-8 antibody triggers higher agonistic activity than bi-epitopic targeting mediated by the combination of two bivalent molecules.

### EXAMPLE 15 MULTIMERIZATION OF TETRA-8+hOX40 CORRELATES WITH IN VITRO ACTIVITY

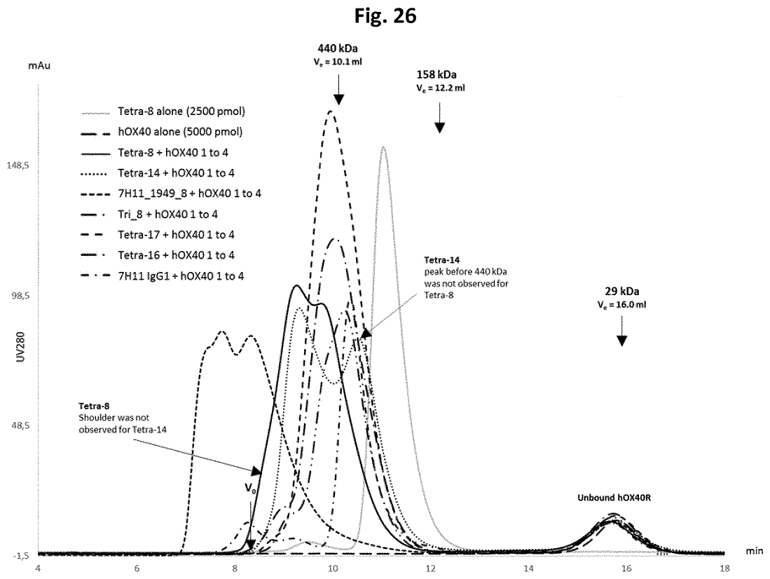

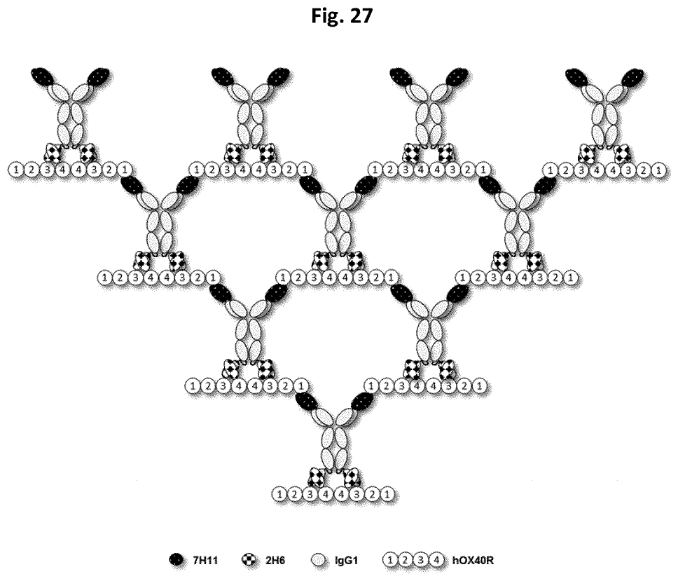

In order to determine the stoichiometry of the Tetra-8+hOX40 complex, analytical gel filtration chromatography was performed on a Superdex 200 10/300 GL increase column connected to an ÄktaPurifier system (both GE Healthcare Europe GmbH, Glattbrugg, Switzerland). Running buffer was PBS pH 7.4 (Gibco, Thermo Fisher Scientific, Reinach, Switzerland) and the flowrate was 0.5 ml/min. Injected sample volumes did not exceed 2% of the column volume and were generally 0.3-0.5 ml. In a first run, 2500 pmol (1 part) of antibody were injected. In a second run, 5000 pmol (2 parts) of hOX40 (SEQ ID NO: 160) were injected. For a third run, 2500 pmol of antibody (1 part) were mixed with 10000 pmol of hOX40 (4 parts) and incubated for 10 minutes at room temperature before injection. A calibration run was performed before using high and low molecular weight calibration kits (28-4038-41, 28-4038-42, GE Healthcare). Chromatograms for Tetra-8 alone, hOX40 alone and Tetra-8/hOX40 at 1:4 ratio are shown in FIG. 26. Rather than observing the peak for the complex minimally shifted towards earlier elution volume in the shape of a single peak, a number of new peaks were observed at significantly higher molecular weight than expected, considering the binding of a 16 kDa ligand. The peaks for the assembly were found eluting earlier than the 440 kDa calibration marker, suggesting complexes were formed that consisted of more than one antibody. The excess of unbound hOX40 of around 5000 pmol (derived from the area under the curve) and the fact that 10,000 pmol were added in the complex mixture, implied that two hOX40 molecules were bound per Tetra-8. Considering the theoretical molecular weights of 198 kDa for Tetra-8 and 16 kDa for hOX40, it could be inferred that the second peak contained 2-3 Tetra-8 molecules in complex with hOX40 and the first peak and its shoulder contained multimers of higher order (complexes composed of more than two antibodies and a number of hOX40). We postulate, that due to its biparatopic nature, Tetra-8 can multimerize with hOX40 to form large crystalline-like lattices (FIG. 27). One hOX40 per Tetra-8 would suffice to create an, in theory, infinitively large lattice, though as mentioned above, two receptors appear to be bound per antibody. A number of control molecules were tested in the same experiment in order to correlate multimerization with in vitro activity. Control molecules containing 7H11 domains only, showed a peak at ˜440 kDa suggesting the formation of unspecific dimers induced by hOX40 binding (FIG. 26, peaks for control antibodies alone are not shown for simplicity). No higher order multimers were observed. In line with this result, no in vitro activity could be observed for these molecules. Tri-8 showed a peak at ˜440 kDa suggesting dimer formation, but no significant amounts of higher order multimers could be observed. Consistent with this result, Tri-8 showed no in vitro activity. Tetra-14 showed two peaks, a first that likely contained higher order multimers and a second peak, eluting before the 440 kDa marker, which may have contained dimers or a single antibody in complex with a number of hOX40 molecules. Tetra-14 showed no activity in vitro, which we hypothesize is the result of its lower propensity for multimerization compared to Tetra-8. Tetra-8 showed no peak at or before the 440 kDa marker but only peaks for higher order multimers. Furthermore, Tetra-8/hOX40 showed a shoulder eluting in the void volume (V0), an observation that could not be made Tetra-14. 7H11_1949_8 showed the highest activity in vitro and at the same time showed the highest magnitude of multimerization in analytical gel filtration compared to any other molecules tested, with most of the protein eluting in V0. 7H11_v8 IgG1 was also included in the experiment and showed a peak at ˜440 kDa, which potentially is the result of unspecific dimer formation. As expected, no peaks for higher order multimerization could be observed for this molecule. Taken together, we propose that the combination of epitope and antibody architecture determines the propensity for multimerization, and higher order multimerization correlates with in vitro activity.

### EXAMPLE 16: TETRA-8 INDUCES LOCAL OX40 CLUSTERING ON CELL SURFACE

### 16.1 Generation of a Stable Jurkat Cell Line Expressing Human OX40-eGFP

Jurkat E6.1 cells (ECACC 88042803) were transfected with pT1-hsOX40-eGFP_fusion_IRES_Puromycin plasmid (GSY935a) using electroporation (Neon® Transfection System), hsOX40-eGFP being a fusion protein with eGFP fused at the C-terminus part of hsOX40. A limiting dilution was done the day after in growth medium containing puromycin (RPMI 1640 with Glutamine+10% FBS+0.25 ug/mL puromycin). After 2 weeks of incubation at 37° C. and 5% CO₂, single pools were analysed for expression of eGFP using Guava® easyCyte flow cytometer and 19 pools were selected. Five days later those pools were analysed for the expression of hsOX40 using FACS and 7 homogenous pools with different expression levels of hsOX40-eGFP fusion protein were kept. They were amplify and frozen in 90% FBS containing 10% DMSO. Expression of OX40-eGFP fusion protein on cell surface allowed a direct visualization of OX40 aggregation upon cellular activation.

### 16.2 Tetra-8 Induces Clustering of Membrane-Bound OX40 on Jurkat OX40-eGFP Cell Line

16.2.1 Time Lapse Confocal Microscopy on OX40-GFP Jurkat Cells Treated with Tetra-8.

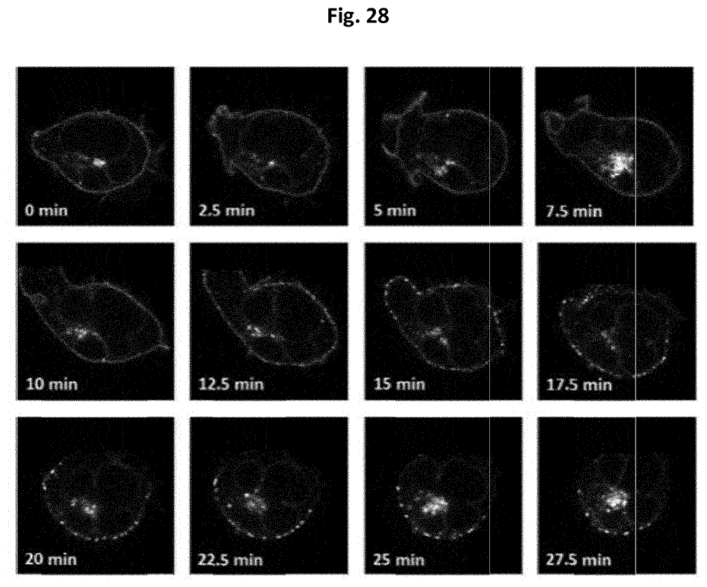

As for other members of TNFRSFs, the activation of downstream signaling depends on OX40 multimerization. The fact that Tetra-8 triggers OX40 signaling in an FcγR-independent way strongly suggests a direct effect of the antibody on OX40 multimerization. In order to interrogate the ability of Tetra-8 to trigger membrane-bound OX40 clustering, a time lapse confocal microscopy experiment was conducted on Jurkat expressing OX40 eGFP cells pre-incubated with Tetra-8. In brief, Fluorodish (WPI) cell culture dishes were pre-coated with 1 mL of fibronectin (at 1 μg/cm2 in PBS) for 45 min at room temperature. Dishes were then washed 2 times with PBS and 3 mL of cell suspension in RPMI and puromycin (20000 cells/cm2) were poured in the dishes. Cells were incubated overnight at 37° C. and 5% CO2. The dishes were placed under the microscope, the focus was set on a typical cell and pictures taken repeatedly every 30 seconds. At time 1.5 min, a solution containing Tetra-8 was added to the medium at 80 nM final concentration. Cells were imaged using a Zeiss Inverted microscope Z1 equipped with a confocal module LSM 800 at 63× magnification. As shown in FIG. 28, following treatment of Jurkat OX40-eGFP cells with Tetra-8, the fluorescence pattern of OX40-GFP switches overtime from a uniform staining of the plasma membrane to a clear aggregation in discrete patches at the plasma membrane.

16.2.2 Testing the OX40-Clustering Induced by Tetra-8 in Comparison with Other OX40-Specific Molecules

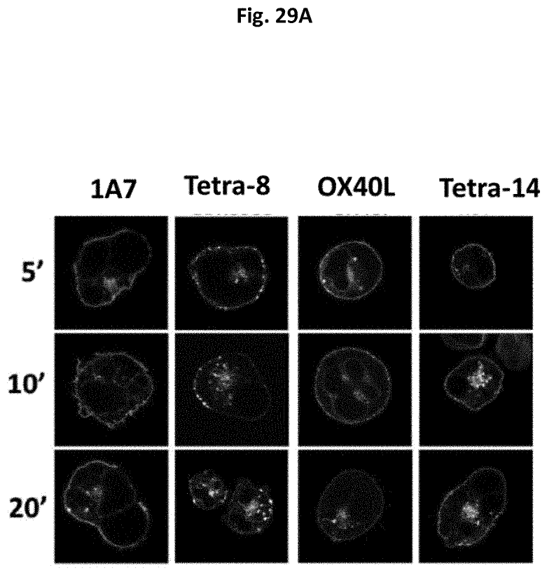

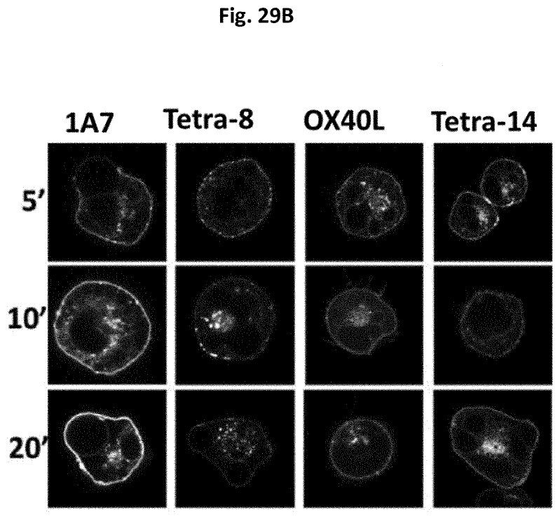

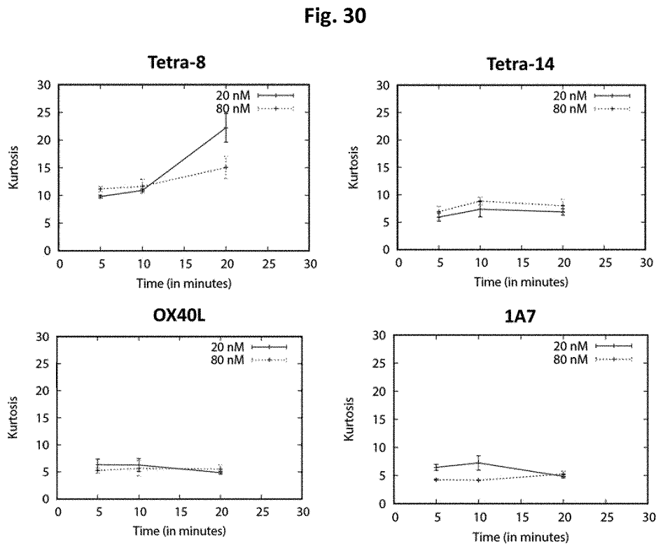

As demonstrated in previous sections, Tetra-8 displays a more potent FcγR-independent agonistic activity than many other monoclonal OX40 agonists. These differences were further investigated in OX40-clustering experiments using fluorescence confocal microscopy. In these experiments, the effect of Tetra-8 in inducing local clustering of membrane-bound OX40 was tested in comparison with Tetra-14, 1A7 and OX40L molecules. In brief, these molecules were tested at two concentrations, 20 and 80 nM, on Jurkat-OX40 eGFP cells, following the protocol described in the previous paragraph. Based on the results from the previous confocal experiment, the timepoints 5, 10 and 20 min were selected to monitor the effect of the tested compounds on OX40 clustering. Results from FIG. 29 show that with either 20 or 80 nM of Tetra-8, the fluorescence pattern of OX40-GFP is significantly affected already after 5 minutes of incubation at 37° C. In comparison, the effect of the other molecules tested is less visible, even after 20 min: 1A7 does not seem to have any qualitative effect, whereas OX40L and Tetra-14 induce very faint concentration of OX40-GFP. In order to evaluate more precisely these differences, a quantitative method was developed. The first step consisted in mapping the cell membrane's fluorescence intensity, based on the 3 dimensional stack of the OX40-GFP fluorescence acquired by confocal microscopy. This fluorescence intensity was displayed in the 0-z coordinate system, where 0 is the angle from an arbitrary point of the cell's membrane with respect to the cell's center and z the height from the coverslip. As a second step, single numerical value was extracted from this fluorescence map: the surface's kurtosis. This parameter, commonly used in surface metrology, was chosen among other because of being a measure of the distribution of spikes above and below the mean line (For spiky surfaces, R_ku>3; for bumpy surfaces, R_ku<3; perfectly random surfaces have kurtosis 3). The kurtosis value was measured for each sample cell resulting in an average kurtosis value with standard error of the mean for each cell experimental condition (i.e. type of drug, drug concentration, time after injection). The standard error from the mean is defined by σ_x=σ/√n with σ the standard deviation of the population and n the number of observations; n was usually equal to 4 cells samples per experimental condition. As shown in FIG. 30, results from the quantitative analysis of OX40 clustering revealed that of the kurtosis values increase over time following treatment of Jurkat-OX40-GFP cells with Tetra-8, compared to other treatments (1A7, OX40L and Tetra-14).

### EXAMPLE 17 TETRAVALENT ANTIBODIES TARGETING DIFFERENT DOMAINS OF CD40

OX40 is a member of the TNFR superfamily which comprises TNFR1, TNFR2, BAFFR, BCMA, TACT, GITR, CD27, 4-1BB, CD40, DR3, HVEM, LTβR, RANK, Fn14, FAS, TRAILR1 and TRAILR2. These receptors are characterized by a common structural domain in their extracellular parts which is the cysteine-rich domain (CRD). Our data showed that the mechanism of action of the tetravalent antibodies which activates OX40 seems to rely on the co-engagement of two different OX40 CRDs. To determine whether this mechanism of action could have a broader application on TNFR superfamily, we have generated anti-CD40 tetravalent molecules as proof of concept. Anti-CD40 antibodies were identified from literature search and their respective sequences were retrieved from patent application or database search. The 2C10 (WO 2017/040932 A1), ADC-1013 (US 2014/0348836 A1), CD40.1 (US 2016/0376371 A1), selicrelumab (U.S. Pat. No. 8,388,971 B2), teneleximab (RCSB, 5DMI) and 3h56-5 (US 2017/0015754 A1) anti-CD40 antibodies were selected as their epitopes on CD40 were known and because their antagonist or agonist activities were also reported. The 2C10, CD40.1, ADC-1013 and Teneleximab are reported to target CD40 CRD1. The selicrelumab binds to the CRD1 and 2 of CD40 while the 3h56-5 interacts with CRD3. The 2C10 and 3h56-5 are described as antagonist while the other antibodies are agonist. Most of these antibodies are targeting CD40 membrane distal domain at the exception of 3h56-5 dAb which is binding to a membrane proximal domain of this receptor. Therefore, anti-CD40 tetravalent molecules were generating by fusing the 3h56-5 dAb sequence to the C-terminus of the heavy chains of 2C10, selicrelumab, CD40.1, ADC-1013 and teneleximab. The VH, VL and dAb sequences of these antibodies were gene synthesized by Geneart AG. The format used for these tetravalent antibodies is similar to tetravalent antibodies described earlier. The VH cDNA sequences were cloned in a modified pcDNA3.1 vector, in frame of a human IgG1-LALA backbone followed by a short G₄T linker sequence and the 3h56-5 dAb sequence. The VL cDNA sequences were cloned in frame of the Kappa or Lambda constant domains in modified pcDNA3.1 vectors. In addition, the selicrelumab and ADC-1013 VH sequences were cloned in frame of a human IgG1 LALA or human IgG1 backbone, respectively, while the 3H56-5 sequence was cloned in frame of a human IgG1 Fc fragments containing the LALA mutation. The tetravalent, selicrelumab IgG1 LALA and 3h56 IgG1 LALA molecules were produced either by co-transfecting the heavy and light chains (table 28) or the single heavy chain in HEK293-EBNA1 cells. The supernatants were collected before protein A purification.

TABLE 28
combination of heavy and light chains for antibody production
Heavy and light chains used for antibody production

| Tetravalent | Heavy chain | Light chain |
|---|---|---|
| 2C10_3h56 | 2C10_3h56 HC | 2C10 LC |
| | (SEQ ID: 162) | (SEQ ID: 167) |
| ADC-1013_3h56 | ADC-1013_3h56 HC | ADC-1013 LC |
| | (SEQ ID: 163) | (SEQ ID: 168) |
| CD40.1_3h56 | CD40.1_3h56 HC | CD40.1 LC |
| | (SEQ ID: 164) | (SEQ ID: 169) |
| selicrelumab_3h5 | selicrelumab_3h56 HC | Selicrelumab LC |
| 6 | (SEQ ID: 165) | (SEQ ID: 220) |
| teneleximab_3h5 | teneleximab_3h56 HC | Teneleximab LC |
| 6 | (SEQ ID: 166) | (SEQ ID: 171) |
| Selicrelumab | selicrelumab IgG1 LALA HC | Selicrelumab LC |
| IgG1 LALA | (SEQ ID: 172) | (SEQ ID: 170) |
| ADC-1013 | ADC-1013 IgG1 HC | ADC-1013 LC |
| IgG1 | (SEQ ID: 173) | (SEQ ID: 168) |
| 3h-56 | 3h-56 IgG1 LALA HC | — |
| IgG1 LALA | (SEQ ID: 174) | |

### EXAMPLE 18: BI-EPITOPIC TARGETING OF CD40 RESULTS IN INCREASED AGONISTIC ACTIVITY

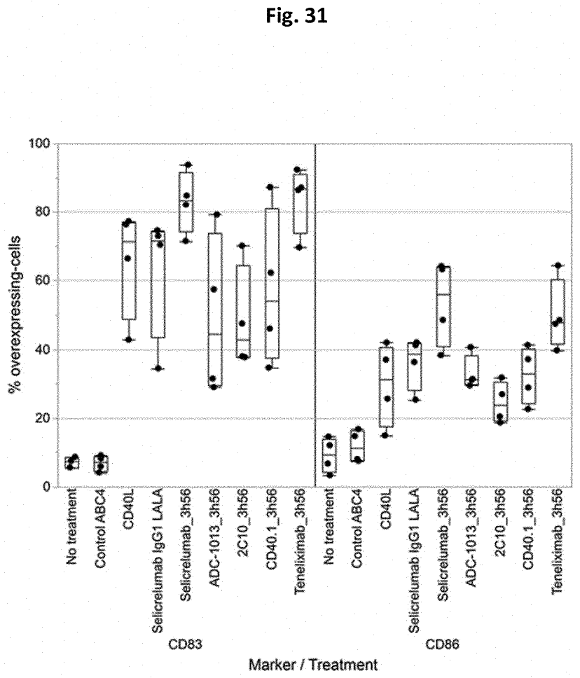

The approach of enhancing OX40 agonism through a bi-epitopic targeting was further extended to CD40, another member of the TNFR superfamily which displays a structural homology with OX40. To this end, various molecules combining binding units derived from anti-CD40 monoclonal antibodies were generated, as listed in table 28, and tested in a DC maturation assay. In this assay, human PBMCs were isolated as described previously and monocytes were purified using a monocyte purification kit, as per the manufacturer's instructions (Stem cell). To generate DCs, purified monocytes were cultured for 6 days at 37° C., 5% CO₂ in the presence of GM-CSF at 50 ng/mL (R&D) and rhIL-4 at 20 ng/mL (R&D) for 6 days. The phenotype of dendritic cells was verified by flow cytometry using CD1c APC (Thermofischer). Cells were then cultured in the presence of anti-CD40 antibodies or controls. Two days later, DC were harvested and stained with anti-CD1c-APC, anti-CD80-PE, anti-CD86-PerCP-eF710 anti-CD83-FITC, anti-HLA-DR-PerCP5.5 (Thermofischer). Cells were washed with 10001 of FACS buffer, and acquired on a Cytoflex. In order to evaluate the potential of the tested anti-CD40 antibodies to upregulate CD83 and CD86 on DC, analysis of the percentage of cells expressing CD83 and CD86 was performed using CytExpert. As shown in FIG. 31, treatment of monocyte-derived DC with either monoclonal monospecific or bi-epitopic anti-CD40 antibodies results in the upregulation of the DC maturation markers CD83 and CD86. Likewise, CD40 and HLA-DR were also upregulated (data not shown). While most of the tested antibodies induce equivalent agonistic effect than soluble CD40L (Selicrelumab IgG1 LALA, ADC-1013_3 h56, 2C10_3 h56 and CD40.1_3 h56), two bi-epitopic anti-CD40 antibodies show even higher agonistic activity (Selicrelumab_3 h56 and Teneliximab_3 h56).

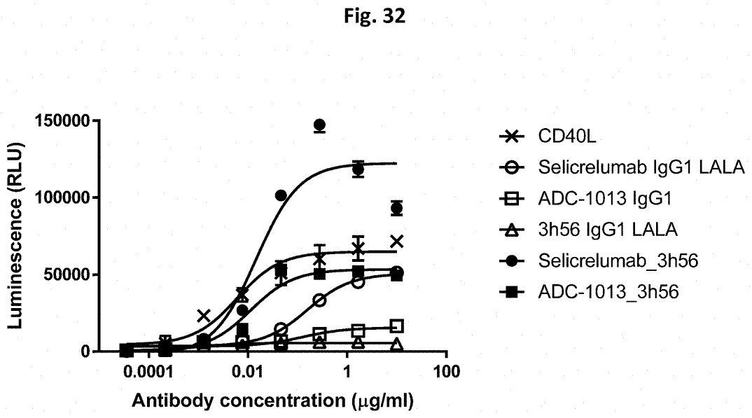

The agonism potential of the anti-CD40 molecules previously tested in DC maturation assay were further evaluated using a CD40-bioassay kit, according to the manufacturer's instructions (Promega). In this assay, NFkB-Luc2P/U20 were resuspended at 3×10⁵ cells/ml in complete RPMI medium (RPMI1640, 10% FBS) and 100 ml of this cell suspension were distributed in 96 luminescence plates. The plates were then incubated overnight at 37° C., 5% CO₂. The following day, all the tested anti-CD40 antibodies were serially diluted in assay buffer (RPMI1640+1% FBS) and 75 ml of this preparation added to the cells. After a 5 hours incubation at 37° C., 5% CO2, 75 μL of Bio-Glo solution were added to the wells and the plates were acquired in a microplate reader. Luminescence was measured using the following settings: read tape—endpoint. Results from FIG. 32 show that the anti-CD40 antibodies display different levels of CD40-dependent luciferase activities. In this assay, 3h56 IgG1 LALA, a reported anti-CD40 antagonistic antibody, does not activate CD40-signals, as expected. However, a combination of its binding units with portions derived from either Selicrelumab (Selicrelumab_3 h56) or ADC-1013 (ADC-1013_3 h56) results in increased activities compared to Selicrelumab or ADC-1013 alone, respectively.

Taken together, results from FIGS. 31 and 32 show that, as observed with OX40, the approach of targeting CD40 using a quadrivalent bi-epitopic antibody promotes enhanced agonistic activities compared to their monospecific counterparts.

1. A TNFR agonist comprising binding portions specific to at least two different parts of said TNFR.

2. The TNFR agonist of claim 1, wherein said TNFR is involved in the costimulation of T cell responses.

3. The TNFR agonist of claim 1, wherein said TNFR is selected from the group consisting of: CD27, 4-1BB (CD137), OX40 (CD134), HVEM, CD30, and GITR.

4. The TNFR agonist of claim 1, wherein said binding portions can bind to said TNFR simultaneously.

5. The TNFR agonist of claim 1, comprising at least two binding portions to each part of said TNFR bound by said agonist.

6. The TNFR agonist of claim 1, wherein said binding portions are selected from the group consisting of antibodies, DARPins, Fynomers, Affimers, variable lymphocyte receptors, anticalin, nanofitin, variable new antigen receptor (VNAR), and derivatives thereof such as a Fab, Fab′, Fab′-SH, Fd, Fv, dAb, F(ab′)2, scFv, Fcabs, bispecific single chain Fv dimers, diabodies, triabodies.

7. The TNFR agonist of claim 5, wherein said at least two binding portions which bind to the same part of said TNFR are disposed at the same peptide terminus of said agonist.

8. The TNFR agonist of claim 1, wherein said binding portions bind to different cysteine-rich domains (CRD) of said TNFR.

9. The TNFR agonist of claim 1, which agonizes OX40 and binds to epitopes in CRD 1 and CRD 3 or CRD 1 and CRD 4 of OX40.

10. The TNFR agonist of claim 9 wherein at least one OX40 binding portion is selected from the group consisting of: SEQ ID NO: 2, 3, 12, 13, 14, 15, 16, 17, 18, 18, 19, 20, 21, 22, 23, 24, 25, 26, 27, 28, 29, 30, 31, 32, 35, 36, 37, 38, 39, 40, 41, 42, 43, 44, 45, 46, 47, 48, 49, and isolated polypeptides having an amino acid sequence that is at least 50%, 60%, 65%, 70%, 75%, 80%, 85%, 90%, 91%, 92%, 93%, 94%, 95%, 96%, 97%, 98%, and 99% identical thereto.

11. An OX40 agonist encoded by SEQ ID Nos: 45 and 16 or isolated polypeptides having an amino acid sequence that is at least 50%, 60%, 65%, 70%, 75%, 80%, 85%, 90%, 91%, 92%, 93%, 94%, 95%, 96%, 97%, 98%, or 99% thereto.

12. A method of treating cancer comprising administering a therapeutically effective amount of the agonist of claim 1 to a subject in need thereof.

13. A method of activating components of the human immune system comprising administering a therapeutically effective amount of the agonist of claim 1 to a subject in need thereof.

14. (canceled)

15. The method of claim 12, wherein the agonist is administered in combination with another medicament.

16. The method of claim 13, wherein the agonist is administered in combination with another medicament.

17. A method of treating cancer comprising administering a therapeutically effective amount of the agonist of claim 11 to a subject in need thereof.

18. A method of activating components of the human immune system comprising administering a therapeutically effective amount of the agonist of claim 11 to a subject in need thereof.
